(19) United States
(12) Patent Application Publication
(10) Pub. No.: US 2020/0352999 A1
(43) Pub. Date: Nov. 12, 2020

(54) USE AND PRODUCTION OF ENGINEERED IMMUNE CELLS TO DISRUPT NFAT-AP1 PATHWAY TRANSCRIPTION FACTORS

(71) Applicant: La Jolla Institute for Allergy and Immunology, La Jolla, CA (US)

(72) Inventors: Anjana RAO, La Jolla, CA (US); James SCOTT-BROWNE, La Jolla, CA (US); Joyce CHEN, La Jolla, CA (US); Hyungseok SEO, La Jolla, CA (US)

(21) Appl. No.: 16/765,783

(22) PCT Filed: Nov. 21, 2018
(86) PCT No.: PCT/US2018/062354

§ 371 Date: May 20, 2020

Related U.S. Application Data
(60) Provisional application No. 62/589,562, filed on Nov. 22, 2017.

(51) Int. Cl. A61K 35/17; C12N 5/0783; C07K 16/28; C07K 14/705; C07K 14/725; A61P 35/00; C12N 5/10

(52) U.S. Cl. CPC A61K 35/17; C07K 2319/40; C07K 16/2803; C07K 14/70521; C07K 14/70517; C07K 14/70578; C07K 14/70575; C07K 14/7051; A61P 35/00; C12N 5/10; C07K 2317/76; C07K 2319/03; C07K 2319/30; C07K 2319/33; C07K 2319/02; C07K 2317/622; C07K 2317/24; C12N 5/0636

(57) ABSTRACT

Immune cells engineered to reduce or eliminate expression and/or the function of a NR4A, TOX, NR4A and a TOX or NR4A and a TOX with increasing expression of IL-21 in said cells are disclosed. Also cells engineered to inhibit expression and/or function of NFAT/AP-1 pathway are provided. Disclosed cells are T and NK cells. It can be expanded to create homogeneous or heterogenous cell populations and/or combined with pharmaceutically acceptable carriers. It can be CAR cells. Methods to induce an immune response and treat conditions requiring selective immunotherapy, comprising contacting a target cell with the cells or compositions as described herein. The contacting can be performed in vitro or in vivo, thereby providing immunotherapy to a subject. Additionally presented herein are methods of producing such engineered cells. Kits containing the materials for making and using the cells are also provided.

### CROSS-REFERENCE TO RELATED PATENT APPLICATION

This application claims priority under 35 U.S.C. § 119(e) to U.S. Provisional Patent Application No. 62/589,562, filed Nov. 22, 2017, the contents of which is hereby incorporated by reference in its entirety.

### STATEMENT OF GOVERNMENT SUPPORT

This disclosure was made with government support from the US National Institutes of Health (AI109842 and AI040127). The government has certain rights in the disclosure.

### FIELD OF THE DISCLOSURE

Embodiments of the disclosure relate to improving adoptive immune cell therapy via the deletion of Nr4a and/or Tox family transcription factors, specifically, T-cell therapy for treatment of cancer or infection.

### BACKGROUND

Adoptive cell therapy for cancer is an increasingly common strategy using infusions of T cells to recognize and eliminate the tumor cells. T cells expressing chimeric antigen receptors (CAR T) targeting human CD19 (huCD19) antigen^{1,2} have exhibited impressive clinical efficacy against B cell leukemias and lymphomas^{3,4}. However, CAR T cells have been less effective against solid tumors^{5,6}, in part because they enter a hyporesponsive⁷ (“exhausted”⁸⁻¹¹ or “dysfunctional”^{12,13}) state that is triggered by chronic antigen stimulation and characterized by upregulation of several inhibitory receptors and loss of effector function^{14,15}. Thus, a need exists in the art to provide immunotherapies targeted to hyporesponsive tumors. This disclosure provides compositions and methods that meet this unmet need.

### SUMMARY

This disclosure provides cells engineered to reduce or eliminate expression and/or the function of a NR4A transcription factor in the cell. Also provided herein are cells engineered to reduce or eliminate expression and/or function of a TOX transcription factor in the cell. In one aspect, this disclosure also presents cells engineered to reduce or eliminate expression and/or function of an NR4A and a TOX transcription factor in the cell. In another aspect, cells are engineered to reduce or eliminate expression and/or function of an NR4A and a TOX transcription factors, and increase expression of IL-21 in the cells. In a further aspect, provided herein are cells engineered to inhibit expression and/or function of NFAT/AP-1 pathway in the cells. For the disclosed cells, in one aspect, the cells are immune cells, such as for example T cells and NK cells.

The cells can be expanded to create homogeneous or heterogeneous cell populations and/or combined with carriers, such as pharmaceutically acceptable carriers.

Also provided herein are methods to induce an immune response and treat conditions requiring selective immunotherapy, comprising, or consisting essentially of, or yet further consisting of, contacting a target cell with the cells or compositions as described herein. The contacting can be performed in vitro, or alternatively in vivo, thereby providing immunotherapy to a subject such as for example, a human patient.

Also presented herein are methods of producing the engineered cells, the methods comprising, or alternatively consisting essentially of, or yet further consisting of, reducing or eliminating expression and/or function of an NR4A transcription factor in the cells.

This disclosure also provides methods of producing engineered cells, the methods comprising reducing or eliminating expression and/or function of a TOX transcription factor in the cells. In another aspect, methods of producing engineered cells, the methods comprising, or alternatively consisting essentially of, or yet further consisting of reducing or eliminating expression and/or function of an NR4A and a TOX transcription factors in the cells. In a further aspect, this disclosure yet further provides methods of producing engineered cells, the methods comprising, or alternatively consisting essentially of, or yet further consisting of reducing or eliminating expression and/or function of an NR4A and a TOX transcription factor, and increasing the expression of IL-21 in the cells. In yet another aspect, provided herein are methods of producing engineered cells, the methods comprising, or alternatively consisting essentially of, or yet further consisting of inhibiting expression and/or function of NFAT/AP-1 pathway in the cells.

For the disclosed methods, in one aspect, the cells are immune cells, such as for example T cells and NK cells.

Kits containing the materials for making and using the cells.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIGS. 1A-1F: Provides a non-limiting example of adoptively transferred chimeric antigen receptor (CAR)—expressing mouse CD8+ T cells exhibiting an exhaustion phenotype similar to that of endogenous CD8+ T cells in a shorter amount of time. FIG. 1A provides a schematic of the tumor experiment. FIG. 1B provides a flow cytometry plot showing populations of CAR CD8+ T cells and endogenous CD8+ T cells on Days 8 post adoptive transfer of CAR T cells. FIG. 1C provides a bar graph showing the percentage of total CD8+ T cells of the two populations. FIG. 1D provides flow cytometry plots showing PD-1 and Tim3 expression of CAR CD8+ T cells or endogenous CD8+ T cells. FIG. 1E provides bar graphs showing surface receptor expression of the four populations (FIGS. 1A-1D). All data are (and will be, for the ones being repeated) mean with data points of three biological replicates, and flow cytometry plots are representative of three biological replicates. FIG. 1F provides flow cytometry plots of cytokine restimulation of CAR CD8+ T cells compared to endogenous CD8+ T cells. Cells were either stimulated with PMA/ionomycin or left unstimulated.

FIGS. 2A-2G: Provides a non-limiting example of tumor-bearing mice adoptively transferred with CAR CD8+ T cells lacking all three Nr4a family members exhibiting increased tumor regression and prolonged survival compared to mice transferred with CAR CD8+ T cells lacking only Nr4a3. FIG. 2A provides a schematic of the tumor experiment. FIG. 2B provides a time course of average tumor growth, n=13 for Nr4a3−/− at d0; n=14 for Nr4aTKO at d0. At d21, the p value was calculated using a t-test assuming equal variances; equal variance determined by f-test. Experiment calculated to have 91% power using a one-sided Mann-Whitney-Wilcoxon Test. FIG. 2C provides corresponding individual mouse tumor growth curves. FIG. 2D shows a survival curve with p value calculated using log-rank (Mantel-Cox) test. FIG. 2E provides a schematic of the tumor experiment. FIG. 2F provides bar graphs showing PD-1, Tim3, Lag3 surface expression of CAR NGFR+ CD8+ T cells on Day 8 post adoptive transfer. Data are mean with data points of two biological replicates. FIG. 2G provides bar graphs showing TNF and IFNγ after restimulation with PMA/ionomycin of CAR NGFR+CD8+ T cells on Day 8 post adoptive transfer. IL-2 not detectable above background in both groups, not shown. Data show mean with data points of two biological replicates.

FIGS. 3A-3G: Adoptively transferred CAR-expressing mouse CD8⁺ T cells and OT-I TCR transgenic T cells infiltrating B16-OVA-huCD19 tumors exhibit phenotypes similar to those of endogenous CD8⁺ TILs. (FIG. 3A) Experimental design to assess the properties of CD45.1⁺ CD8⁺ CAR-expressing and endogenous CD45.2⁺ TILs isolated 21 days after tumor injection and 8 days after adoptive transfer of 1.5 million CART cells. (FIG. 3B) Left, flow cytometry plot showing populations of CAR CD8⁺ TILs and endogenous CD8⁺ TILs; CAR TILs by CD45.1⁺ expression as well as expression of Thy1.1 encoded in the CAR retroviral vector. Right, flow cytometry plots showing PD-1 and TIM3 surface expression on CAR CD8⁺ TILs and endogenous CD8⁺ TILs. (FIGS. 3C-3D) Experimental design to assess the properties of CD45.1⁺ OT-I TCR-transgenic TILs; other details as in (FIG. 3B). (FIG. 3E) Bar graph showing the percentage of CAR and OT-I TILs in total CD8+ TILs. Bars show mean values with data points for 6, 5 and 11 biological replicates for CAR, OT-I and endogenous TILs respectively. Flow cytometry plots are representative of all biological replicates. (FIG. 3F, FIG. 3G) Quantification of cytokine production after restimulation of CAR, OT-I and endogenous CD8⁺ TILs, compared to CD8⁺ T cells retrovirally transduced with the CAR and restimulated in vitro. Cells were stimulated with PMA/ionomycin or left unstimulated. (FIG. 3F) Bars shown are mean values with data points for three biological replicates. All p values were calculated using an unpaired t test with Welch's correction. * p≤0.05, ** p≤0.01, *** p≤0.001, **** p≤0.0001. (FIG. 3G) Representative flow cytometry plots of cytokine production after restimulation (data from (FIG. 3F)).

FIGS. 4A-4F: Gene expression and chromatin accessibility profiles of PD-1hiTIM3hi CAR CD8⁺ TILs resemble those of PD-1hiTIM3hi endogenous CD8+ TILs. (FIG. 4A) Principle component analysis (PCA) of RNA sequencing (RNA-seq) data from the various populations of CAR-expressing TILs, CAR TILs PD-1hiTIM3hi (FIG. 4A) and PD-1hiTIM3lo (FIG. 4B) populations, and the endogenous TILs PD-1hiTIM3hi (FIG. 4C), PD-1hiTIM3lo (FIG. 4D), PD-1loTIM3lo (FIG. 4E) populations. Percentage of variance of PC1 and PC2 indicated. (FIG. 4B) Top, heatmap of mouse CD8+ T cell ATAC-seq data showing log 2 fold change from row mean for 9 clusters as determined by k-means clustering. Bottom, heatmap of motif enrichment analysis. Data for one representative member of transcription factor families enriched in at least one cluster compared to all accessible regions are shown. (FIG. 4C) Representative flow cytometry plots for protein level expression of Nr4a1, Nr4a2, and Nr4a3 compared between the CAR TILs PD-1hiTIM3hi (FIG. 4A) and PD-1hiTIM3lo (FIG. 4B) populations, and between the endogenous TILs PD-1hi− TIM3hi (FIG. 4C), PD-1hiTIM3lo (FIG. 4D), PD-1loTIM3lo (FIG. 4E); (FIG. 4D) Quantification of Nr4a expression levels; data show mean and individual values from three independent biological replicates. (FIG. 4E) Scatterplots of single cell RNA-seq of human CD8⁺ TILs²⁰, plotting in single cells the expression of PDCD1 and HAVCR2 (x and y axis, respectively), and the expression of the different NR4A genes (color scale). Each dot represents a single cell. (FIG. 4F) Top, human CD8+ T cell ATAC-seq data from PD-1hi TILs, two samples from human melanoma and one sample from a non-small cell lung cancer (NSCLC) tumor¹⁹, and antigen-specific CD8+ T cells from HIV-infected individuals²¹ showing log 2 fold change from row mean for 9 clusters as determined by k-means clustering. Bottom, heatmap of motif enrichment analysis.

FIGS. 5A-5F: Tumor-bearing mice adoptively transferred with Nr4a TKO CAR CD8+ T cells lacking all three Nr4a family members exhibit increased tumor regression and prolonged survival compared to mice transferred with wildtype CAR CD8⁺ T cells. (FIG. 5A) Experimental design for monitoring tumor growth; adoptive transfer of 3 million CAR T cells on day 7 after tumor inoculation. (FIG. 5B) Top three graphs, time course of tumor growth in individual mice, comprised of 30 or more biological replicates per condition. At d7, mouse numbers were n=21 for PBS, n=35 for WT and n=39 for Nr4aTKO. Bottom, at day 21, mouse numbers were n=14 for PBS, n=25 for WT, n=36 for Nr4aTKO; data show mean±s.d. and the individual values; p values were calculated using an ordinary one-way ANOVA with Tukey's multiple comparisons test; * p=0.0331, **** p<0.0001. (FIG. 5C) p value for survival curve was calculated using log-rank (Mantel-Cox) test; **** p<0.0001. At d90, the numbers of surviving mice were n=0 for PBS, n=1 for WT and n=27 for Nr4aTKO. (FIG. 5D) Experimental design for assessing the properties of CD8⁺ TILs; adoptive transfer of 1.5 million CART cells on day 13 after tumor inoculation. (FIG. 5E) PD-1 and TIM3 expression in WT and Nr4aTKO TILs 8 days after adoptive transfer. Both samples are gated on cells with a set level of CAR expression (10³-10⁴) within the CAR⁺ NGFR⁺ population. Top panels, representative flow cytometry plots of PD-1 and TIM3 surface expression; Middle and bottom left, representative flow cytometry histograms of PD-1 and TIM3 expression. The means and individual values of six independent experiments are shown, for which each point represents TILs from a pool of 3-8 mice. Two-tailed p values were calculated using paired t tests with Welch's correction. (FIG. 5F) Cytokine production by WT and Nr4aTKO TILs 8 days after adoptive transfer. Top, flow cytometry plots showing TNF and IFNγ production by representative CAR⁺ NGFR⁺ CD8+ TILs left unstimulated or stimulated with PMA/ionomycin. Bottom, bar graphs showing TNF and IFNγ production individually and together after restimulation with PMA/ionomycin of CAR NGFR⁺ CD8⁺ T cells on day 8 after adoptive transfer. IL-2 was not detectable above background in either group (not shown). The means and individual values of five independent experiments are shown, for which each point represents TILs from a pool of 3-8 mice. Two-tailed p values were calculated using a paired t test between the unstimulated samples and between the stimulated samples for WT and Nr4aTKO; * p≤0.05, ** p≤0.01, *** p≤0.001, **** p≤0.0001.

FIGS. 6A-6F: Gene expression and chromatin accessibility profiles of the Nr4a TKO CAR CD8+ TILs indicate increased effector function compared to the WT CD8⁺ TILs. (FIG. 6A) Mean average (MA) plots of genes differentially expressed in Nr4a TKO relative to WT CAR TILs. Genes differentially expressed (adjusted p value<0.1 and log 2FoldChange≥1 or ≤−1) are highlighted using different colors as indicated in the color key. Selected genes are labeled. (FIG. 6B) Heatmap of genes with opposing expression changes between Nr4a deletion and Nr4a overexpression. Fold change values (log 2 scale) of all genes differentially expressed in Nr4a TKO relative to WT CAR TILs were compared to the corresponding gene values in cells ectopically expressing Nr4a1, Nr4a2, and Nr4a3. Different clusters were identified by the k-means method (k=7). Highlighted are genes downregulated after Nr4a deletion and upregulated after Nr4a overexpression (e.g. Pdcd1, Havcr2, and Tox), and genes upregulated after Nr4a deletion and downregulated after Nr4a overexpression (e.g. Tnf and Il21). (FIG. 6C) Scatterplot of pairwise comparison of ATAC-seq density (Tn5 insertions per kb). Differentially accessible regions, and associated de novo identified motifs, between Nr4a TKO and WT CAR TILs are indicated. (FIG. 6D) Left, genome browser view of the Pdcd1 locus incorporating all previously mentioned ATAC-seq samples, and from cells transduced with CA-RIT-NFAT1. The gray bar shows the exhaustion-specific enhancer located ˜23 kb 5′ of the transcription start site of Pdcd1. Right top, histogram view of showing expression of Nr4a in cells ectopically expressing HA-tagged versions of Nr4a1, Nr4a2, Nr4a3. Right bottom, bar plot showing enrichment of chromatin-immunoprecipitated HA-tagged Nr4a over background at the exhaustion-specific enhancer located ˜23 kb 5′ of the transcription start site of Pdcd1. (FIG. 6E) Comparison of ATAC-seq data from Nr4a TKO and WT CAR TILs with those from cells ectopically expressing (from top towards bottom) either CA-RIT-NFAT1, Nr4a1, Nr4a2, or Nr4a3. Bottom panel, comparisons of ATAC-seq data from Nr4a TKO and WT CAR TILs with those from cells that have been stimulated with PMA/ionomycin. (FIG. 6F) Schematic illustrating proposed role of Nr4a in T cells undergoing chronic antigen stimulation.

FIGS. 7A-7H: Surface expression and functional assessment of a chimeric antigen receptor (CAR) reactive to human CD19 in mouse CD8⁺ T cells. (FIG. 7A) Cell lines expressing huCD19. Left, EL4 cells; gray=parent EL4, black=EL4 expressing huCD19. Middle left, MC38 cells; gray=parent MC38, black=MC38 expressing huCD19. Middle right, B16-OVA cells; gray=parent B16-OVA, black=B16-OVA expressing huCD19. Right; B16-OVA-huCD19 cells in vivo; gray=isotype control, black=B16-OVA cells expressing huCD19 isolated from a C57BL/6J tumor-bearing mouse and cultured for 7 days after isolation. (FIG. 7B) Left, tumor growth curves showing comparison of inoculation of 250K B16-OVA parent tumor cells or 250K B16-OVA-huCD19 tumor cells; n=15 for both groups. Ordinary two-way ANOVA shows no significant difference at any timepoint between the two groups. Right; tumor growth curves showing comparison of inoculation of 250K or 500K B16-OVA-huCD19 tumor cells; n=5 for 250K, n=6 for 500K. Ordinary two-way ANOVA shows a significant difference between the two groups at day 21; *p=0.0146. (FIG. 7C) Schematic of the CAR construct. LS=leader sequence; SS=signal sequence; myc=myc-tag; scFV=the single chain fragment variable against human CD19; followed by the mouse CD28, mouse CD3ζ signaling domains; and the 2A self-cleaving peptide and mouse Thy1.1 reporter. (FIG. 7D) Surface expression of the CAR, monitored as expression of the Thy1.1 reporter or myc tag. Mock-transduced CD8+ T cells used as control. (FIG. 7E) Flow cytometry plots showing cytokine production (TNF and IFNγ) of CAR-expressing CD8⁺ T cells after restimulation with EL4 cells expressing huCD19 or PMA/ionomycin. Data are representative of three independent biological replicates. (FIG. 7F) Bar graphs of the data shown in (e); data are from three independent biological replicates; p values were calculated using a two-tailed unpaired t test. * p≤0.05, ** p≤0.01, *** p≤0.001, **** p≤0.0001. (FIG. 7G) In vitro killing assay of CD8+ CAR T cells compared to mock transduced CD8⁺ T cells; p value was calculated using an ordinary two-way ANOVA. (FIG. 7H) Inhibitory surface receptor expression on CD8⁺ T cells transduced with CAR or mock transduced and cultured in vitro for 5 days; data are representative of three independent biological replicates. Gray shading=isotype control, black line=mock or CAR.

FIGS. 8A-6C: Adoptively transferred CAR-expressing mouse CD8⁺ T cells and OT-I TCR transgenic T cells infiltrating B16-OVA-huCD19 tumors exhibit phenotypes similar to those of endogenous CD8⁺ TILs. (FIG. 8A, FIG. 8B) Flow cytometry gating scheme for CAR (FIG. 8A) and OT-I (FIG. 8B) CD8⁺ TILs. (FIG. 8C) Top, tumor growth curves for tumor-bearing mice adoptively transferred with CAR or OT-I CD8+ T cells; graph is a compilation of 3 independent experiments. At d=7, CAR n=24, OT-I n=21; at d=21, CAR n=17, OT-I n=20. Bottom, tumor growth curves for tumor-bearing mice adoptively transferred with CAR or PBS; graph is a compilation of 3 independent experiments. At d=7, CAR n=35, PBS n=8. At d=21, CAR n=35, PBS n=6. Tumor size on day 21 after tumor inoculation shows p value=0.3527 for CAR compared to OT-I (top); p value=0.6240 for PBS compared to CAR (bottom); these values were calculated using a two-tailed unpaired-t test with Welch's correction.

FIG. 9: Comparisons of the gene expression profiles of the CAR-expressing and endogenous CD8⁺ TILs. Mean average (MA) plots of genes differentially expressed in the indicated comparisons; genes differentially expressed (adjusted p value<0.1 and log 2FoldChange≥1 or ≤−1) are highlighted using different colors as indicated in the key. Selected genes are labeled. Top row, comparisons of the CAR TIL populations amongst themselves and to the endogenous PD-110 TIM3lo TILs; middle row, comparisons within the endogenous TIL populations; bottom row, comparisons of CAR and endogenous PD-1hi TIM3hi TILs (left), and CAR and endogenous PD-1hi TIM3lo TILs (right).

FIGS. 10A-10C: Comparisons of the chromatin accessibility profiles of the CAR, endogenous, and OT-I CD8⁺ TILs. (FIG. 10A) Pairwise euclidean distance comparisons of log 2 transformed ATAC-seq density (Tn5 insertions per kilobase) between all replicates at all peaks accessible in at least one replicate. (FIG. 10B) Scatterplot of pairwise comparison of ATAC-seq density (Tn5 insertions per kb) between samples indicated. (FIG. 10C) Genome browser views of sample loci, Pdcd1 (left), Itgav (right); scale range is from 0-1000 for all tracks and data are the mean of all replicates. CD8⁺ TIL populations are as indicated and defined in FIG. 1B, FIG. 1D: (A) PD-1hi TIM3hi CAR, (B) PD-1hi TIM3lo CAR, (C) PD-1hi TIM3hi endogenous, (D) PD-1hi TIM3lo endogenous, (E) PD-110 TIM3lo endogenous.

FIGS. 11A-11B: Endogenous mouse CD8⁺ T cells and adoptively transferred CAR-expressing mouse CD8⁺ T cells infiltrating B16-OVA-huCD19 tumors exhibit increased levels of Nr4a. (FIG. 11A) Flow cytometry gating scheme for CAR (left) and endogenous (right) CD8⁺ TILs. (FIG. 11B) Representative flow cytometry histograms of Nr4a proteins in PD-1hi TIM3hi TILs, PD-1hi TIM3lo TILs, and PD-1loTIM3lo TILs and their corresponding fluorescence minus one control (in off-white).

FIGS. 12A-12C: Scatterplots of single cell RNA-seq of human CD8⁺ TILs. Plotting in single cells the expression of PDCD1 and HAVCR2 (x and y axis respectively), and (displayed by the color scale) the expression of (FIG. 12A) Genes differentially upregulated in PD-1hi TIM3hi TILs relative to PD-110 TIM3lo TILs. (FIG. 12B) Genes differentially downregulated in PD-1hi TIM3hi TILs relative to PD-110 TIM3lo TILs. (FIG. 12C) Genes coding for selected transcription factors showing differential expression in the comparison of PD-1hi TIM3hi TILs relative to PD-1hi TIM3lo TILs. Each dot represents a single cell. Human CD8⁺ TILs data are from²⁰.

FIGS. 13A-13C: Robust double transduction efficiency to produce WT and Nr4a TKO CAR T cells for adoptive transfer. (FIG. 13A) CD8a only staining control (previously tested to be the same as fluorescence minus one control for CAR^{P} expression and NGFR⁺ expression) of CAR T cells prior to adoptive transfer. (FIG. 13B) CAR and NGFR expression of CD8+WT CAR T cells prior to adoptive transfer. (FIG. 13C) CAR and NGFR expression of CD8+Nr4a TKO CAR T cells prior to adoptive transfer.

FIGS. 14A-14D: Tumor-bearing mice adoptively transferred with CAR CD8+ T cells lacking all three Nr4a family members exhibit prolonged survival compared to mice transferred with wildtype CAR CD8⁺ T cells or CAR CD8⁺ T cells lacking only one of the three Nr4a family members. (FIG. 14A) Experimental design for monitoring tumor growth; adoptive transfer of 3 million CAR T cells on day 7 after tumor inoculation. (FIG. 14B) Time course of tumor growth in individual mice bearing B16-OVA-huCD19 tumors, comprised of 17 or more biological replicates per condition (these data include the WT and Nr4a TKO data from FIG. 3). At d7, mouse numbers were n=31 for PBS, n=35 for WT, n=17 for Nr4a1 KO, n=17 for Nr4a2 KO, n=32 for Nr4a3−/−, and n=39 for Nr4a TKO. At day 21, mouse numbers were n=14 for PBS, n=25 for WT, n=12 for Nr4a1 KO, n=15 for Nr4a2 KO, n=22 for Nr4a3−/−, and n=36 for Nr4a TKO. (FIG. 14A) Graph shows mean±s.d. and the individual values of B16-OVA-huCD19 tumor sizes at day 21 after inoculation. p values were calculated using an ordinary one-way ANOVA with Tukey's multiple comparisons test; PBS vs WT, * p=0.0395; WT vs Nr4a1KO, p=n.s.=0.0511; WT vs Nr4a2KO, ** p=0.002, WT vs Nr4a3−/−, * p=0.0161; and WT vs Nr4a TKO, **** p<0.0001. (d) Survival curves for mice. p value for survival curve was calculated using log-rank (Mantel-Cox) test; **** p<0.0001. At d90, mouse numbers were n=0 for PBS, n=1 for WT, n=0 for Nr4a1 KO, n=1 for Nr4a2 KO, n=11 for Nr4a3−/−, and n=27 for Nr4a TKO.

FIGS. 15A-15D: Ectopic expression of Nr4a1, Nr4a2, Nr4a3 in mouse CD8+ T cells results in decreased cytokine production and increased expression of inhibitory surface markers. Mouse CD8+ T cells were isolated, activated, and transduced with empty vector or HA-tagged Nr4a1, Nr4a2, or Nr4a3 vectors with human NGFR reporter. Cells were assayed on day 5 post activation. (FIG. 15A) Flow cytometry gating of CD8+ NGFR+ empty vector control, Nr4a1, Nr4a2, and Nr4a3-expressing cells at a constant expression level of NGFR reporter. (FIG. 15B) Quantification of surface receptors expression (data are from three independent biological replicates). (FIG. 15C) Top, representative flow cytometry plots of cytokine production upon restimulation with PMA/ionomycin; bottom, quantification of the cytokine production after restimulation (data are from three independent biological replicates). All p values were calculated using an ordinary one-way ANOVA with Dunnett's multiple comparisons test; * p≤0.05, ** p≤0.01, *** p≤0.001, **** p≤0.0001. (FIG. 15D) Quantification of transcription factors (data from three independent biological replicates).

FIGS. 16A-16B: Comparison of the gene expression profiles of the mouse CD8+ T cells ectopically expressing Nr4a1, Nr4a2, Nr4a3 in vitro. (FIG. 16A) Principle component analysis (PCA) of RNA sequencing (RNA-seq) data from the mouse CD8⁺ T cells ectopically expressing Nr4a1, Nr4a2, Nr4a3, and empty vector control, in vitro. Percentage of variance of PC1 and PC2 indicated. (FIG. 16B) Mean average (MA) plots of genes differentially expressed in the comparisons of ectopic expression of Nr4a1, Nr4a2, or Nr4a3 against empty vector (top row), and pairwise comparisons between the ectopic expression of various Nr4a family members (bottom row). Genes differentially expressed (adjusted p value<0.1 and log 2 FoldChange≥1 or ≤−1) are highlighted using different colors as indicated in the PCA plot as in (a). Selected genes are labeled.

FIG. 17: Comparison of the chromatin accessibility profiles of the mouse CD8+ T cells ectopically expressing Nr4a1, Nr4a2, Nr4a3 in vitro. Scatterplot of pairwise comparison of ATAC-seq density (Tn5 insertions per kb) between samples indicated.

FIGS. 18A-18D: Phenotyping of TILs isolated from tumor-bearing mice adoptively transferred with Nr4a TKO CAR CD8⁺ T cells lacking all three Nr4a family members or WT CAR CD8⁺ T cells. (FIG. 18A) Tumor growth of delayed adoptive transfer of 1.5 million CART cells on day 13 after tumor inoculation; TILs were isolated from these tumors for phenotyping. Tumor growth curves for Rag1−/− mice adoptively transferred with WT and Nr4aTKO CAR CD8+ T cells; at d=7, WT=47 and Nr4aTKO=41; at d=21, WT=35 and Nr4aTKO=32. p values were calculated using an ordinary 2-way ANOVA with Tukey's multiple comparisons test; for WT vs Nr4aTKO, p value=n.s.=0.6908. (FIG. 18B) Flow cytometry gating scheme for assay of surface markers, cytokines, and transcription factors expressed by WT (top) and Nr4aTKO (bottom) TILs. All samples are gated on cells with a set level of CAR expression (10³-10⁴) within the CAR+ NGFR⁺ population. (FIG. 18C) Bar plots of TIL counts and MFI of Ki67 of WT and Nr4aTKO CAR TILs. (FIG. 18D) Top, bar plots of transcription factors expression of WT and Nr4aTKO CAR TILs. Bottom, representative flow cytometry plots of TIM3 and TCF1 expression in WT and Nr4aTKO CAR TILs. Two-tailed p values were calculated using paired t tests. * p≤0.05, ** p≤0.01, *** p≤0.001, **** p≤0.0001.

FIGS. 19A-19C: Gene expression of the Nr4a TKO CAR CD8⁺ TILs indicate increased effector function compared to the WT CAR CD8+ TILs. (FIG. 19A) Principle component analysis (PCA) of RNA sequencing (RNA-seq) data from the Nr4a TKO CAR TILs or the WT CAR TILs. Percentage of variance of PC1 and PC2 indicated. (FIG. 19B) Normalized enrichment scores (NES) of gene sets defined from pairwise comparisons of effector, memory and exhausted CD8⁺ T cells from LCMV-infected mice¹⁷. (FIG. 19C) Gene set enrichment analysis of RNA-seq data from Nr4a TKO CAR and WT CAR TILs displayed as enrichment plots, ranking genes by log 2 fold change in expression between those conditions.

FIGS. 20A-20B: Nr4a family members bind to predicted Nr4a binding motifs that are more accessible in the WT CAR TILs compared to the Nr4a TKO CAR TILs. (FIG. 20A) Right top, representative histogram view showing expression of Nr4a in cells ectopically expressing HA-tagged versions of Nr4a1, Nr4a2, Nr4a3. Middle, genome browser views of the Ccr7, Ccr6, Ifng loci for WT CAR TILs compared to Nr4a TKO CAR TILs, including binding motifs for NFAT, Nr4a (Nur77), bZIP, NFκB, and the location of the qPCR primers used. Scale range is 0-1000 for all tracks and data are mean of all replicates. Right, bar plots showing enrichment of Nr4a at regions probed. (FIG. 20B) Genome browser views of the Il21 (left), Tnf (right) locus incorporating WT CAR TILs compared to Nr4a TKO CAR TILs, including binding motifs for NFAT, Nr4a (Nur77), bZIP, NFκB. Scale range is 0-1000 for all tracks and data are mean of all replicates.

FIG. 21: Nr4a family members show a moderate decrease in mRNA expression in antigen-specific cells from LCMV-infected mice treated with anti-PDL1 or IgG control. Mean average (MA) plots of genes differentially expressed in cells treated with anti-PDL1 treatment compared to cells treated with IgG control, highlighting two different categories of differentially expressed genes: those with adjusted p value<0.1 and log 2FoldChange≥0.5 or ≤−0.5 (lighter colors); and those with adjusted p value<0.1 and log 2FoldChange≥1 or ≤−1 (darker colors). Selected genes are labeled. Displayed are the number of genes in each category. The sequencing data in this analysis was obtained from²⁹.

FIGS. 22A-22D: Immunocompetent tumor-bearing mice adoptively transferred with Nr4a TKO CAR CD8⁺ T cells lacking all three Nr4a family members exhibit prolonged survival compared to mice transferred with WT CAR CD8+ T cells. (FIG. 22A) Experimental design for monitoring tumor growth; adoptive transfer of 6 million CAR T cells on day 7 after tumor inoculation. (FIG. 22B) Left three graphs, time course of tumor growth in individual mice bearing B16-OVA-huCD19 tumors, comprised of 13-15 biological replicates per condition. At d7, mouse numbers were n=13 for PBS, n=15 for WT and n=14 for Nr4a TKO. At day 21, mouse numbers were n=11 for PBS, n=11 for WT, n=13 for Nr4a TKO. Right three graphs, time course of tumor growth in individual mice bearing MC38-huCD19 tumors, comprised of 10 biological replicates per condition. At d7, mouse numbers were n=10 for PBS, n=10 for WT and n=10 for Nr4a TKO. At day 19, mouse numbers were n=9 for PBS, n=7 for WT, n=10 for Nr4a TKO. (FIG. 22C) Left, graph shows mean±s.d. and the individual values of B16-OVA-huCD19 tumor sizes at day 21 post inoculation; p values were calculated using an ordinary one-way ANOVA with Tukey's multiple comparisons test and show no significance. Right, graph shows mean±s.d. and the individual values of MC38-huCD19 tumor sizes at day 19 post inoculation time; p values were calculated using an ordinary one-way ANOVA with Tukey's multiple comparisons test; * p=0.012, *** p=0.0001. (FIG. 22D) Survival curves for mice bearing B16-OVA-huCD19 tumors (left) and MC38-huCD19 tumors (right). p value for survival curve was calculated using log-rank (Mantel-Cox) test; **** p<0.0001. For mice bearing B16-OVA-huCD19 tumors at d90, mouse numbers were n=0 for PBS, n=0 for WT and n=2 for Nr4a TKO; * p=0.0026. For mice bearing MC38-huCD19 tumors, all mice died by d23; * p=0.0138.

FIGS. 23A-23B: Tox and PD-1 is highly expressed in CAR-T cell in solid tumors. Experimental scheme: CAR-T cells were adoptive transferred 12 days after B16-huCD19 tumor injection and CD45.1+CD8+ CAR expressing TILS isolated 16, 20 and 25 days. PD-1 and TOX expression levels analyzed by flow cytometry from CAR-T cell (Day 12: Before transfer), and CAR-expressing TILS (Day 16, 20 and 24) (FIG. 23A-B).

FIG. 24: C57BL/6N mice bearing B16-huCD19 tumors adoptively transferred with TOX/TOX2 lacking CAR-T cells inhibit tumor growth and prolonged survival compared to mice transferred WT CAR-T cells 3 million CAR-T cells are adoptively transferred on Day 7 after tumor inoculation. Tumor size were measured by caliper every 2 days.

FIG. 25: Time course of tumor growth in individual mice and survival curve.

FIG. 26: Experimental scheme: TOX and TOX 2 lacking CAR-T cells or WT CAR T cells were adoptive transferred 12 days after B16-huCD19 tumor injection and CD45.1+CD8+ CAR expressing TILS isolated on Day 24 PD-1, Tim-3, Lag-3 and CD160 expression levels analyzed by flow cytometry from CAR expressing TILS.

FIG. 27: Experimental scheme: TOX and TOX 2 lacking CAR-T cells or WT CAR T cells were adoptive transferred 12 days after B16-huCD19 tumor injection and CD45.1+CD8+ CAR expressing TILS isolated on Day 24 IFN-gamma and TNF-alpha expression levels analyzed by flow cytometry from CAR expressing TILS after PMA/Ionomycin stimulation (4 hours).

FIG. 28: Experimental scheme: TOX and TOX 2 lacking CAR-T cells or WT CAR T cells were adoptive transferred 12 days after B16-huCD19 tumor injection and CD45.1+CD8+ CAR expressing TILS isolated on Day 24 T-bet and Eomes expression levels analyzed by flow cytometry from CAR expressing TILS.

FIG. 29: RAG1−/− mice bearing B16-huCD19 tumors adoptively transferred with TOX/TOX2 lacking CAR-T cells inhibit tumor growth and prolonged survival compared to mice transferred WT CAR-T cells. 3 million CAR-T cells are adoptively transferred on Day 7 after tumor inoculation. Time course of tumor growth in individual mice and survival curve. Tumor size were measured by caliper every 2 days.

FIG. 30: Plasmid map of plasmid JC31 carrying the CAR-2A-Thy 1.1 construct.

FIG. 31: Plasmid sequence of the plasmid JC31 carrying the CAR-2A-Thy 1.1 construct with the following elements: MESV located between 579 and 920, gag (truncated) located between 987 and 1403, Kozak sequence located between 1416 and 1425, Myc located between 1485 and 1514, CAR located between 1515 and 2900, P2A located between 2901 and 2957, mouse Thy 1.1 located between 2958 and 3446, 3′ LTR located between 3496 and 3010, M13 rev located between 4179 and 4195, lac operator located between 4203 and 4219, lac promoter located between 4220 and 4271, CAP binding site located between 4272 and 4293, ori located between 4581 and 5169, AmpR located between 5340 and 6200, and AmpR promoter located between 6201 and 6305.

### DETAILED DESCRIPTION

It is to be understood that the present disclosure is not limited to particular aspects described, as such may, of course, vary. It is also to be understood that the terminology used herein is for the purpose of describing particular embodiments only, and is not intended to be limiting, since the scope of the present disclosure will be limited only by the appended claims.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

It is to be inferred without explicit recitation and unless otherwise intended, that when the present technology relates to a polypeptide, protein, polynucleotide or antibody, an equivalent or a biologically equivalent of such is intended within the scope of the present technology.

Throughout this disclosure, various publications, patents and published patent specifications are referenced by an identifying citation. The full bibliographic information for the citations is found immediately preceding the claims. All publications, patent applications, patents, and other references mentioned herein are expressly incorporated by reference in their entirety, to the same extent as if each were incorporated by reference individually. In case of conflict, the present specification, including definitions, will control.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The disclosure is generally disclosed herein using affirmative language to describe the numerous embodiments and aspects. The disclosure also specifically includes embodiments in which particular subject matter is excluded, in full or in part, such as substances or materials, method steps and conditions, protocols, or procedures. For example, in certain embodiments or aspects of the disclosure, materials and/or method steps are excluded. Thus, even though the disclosure is generally not expressed herein in terms of what the disclosure does not include aspects that are not expressly excluded in the disclosure are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

### Definitions

As used herein, the singular forms “a”, “an,” and “the” include plural referents unless the context clearly indicates otherwise. Thus, for example, reference to “an NR4A transcription factor” or “a TOX transcription factor” includes a plurality of such NR4A or TOX transcription factors.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

As used herein “a population of cells” intends a collection of more than one cell that is identical (clonal) or non-identical in phenotype and/or genotype.

As used herein, “substantially homogenous” population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers, phenotypic or genomic traits. In one aspect, the population is a clonal population.

As used herein, “heterogeneous” population of cells is a population having up to 69%, or alternatively up to 60%, or alternatively up to 50%, or alternatively up to 40%, or alternatively up to 30%, or alternatively up to 20%, or alternatively up to 10%, or alternatively up to 5%, or alternatively up to 4%, or alternatively up to 3%, or alternatively up to 2%, or alternatively up to 61%, or alternatively up to 0.5% identical phenotype, as measured by pre-selected markers, phenotypic or genomic traits.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies. In one aspect, treatment excludes prophylaxis.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

As used herein, “anti-tumor immunity” in a subject refers to reducing or preventing the symptoms or cancer from occurring in a subject that is predisposed or does not yet display symptoms of the cancer.

As used herein, providing “immunity to the pathogen infection” in a subject refers to preventing the symptoms or pathogen infection from occurring in a subject that is predisposed or does not yet display symptoms of the pathogen infection. In one aspect, immunity reduces or diminishes the course or severity of symptoms and duration of the infection.

In some embodiments a subject is in need of a treatment, cell or composition described herein. In certain embodiments a subject has or is suspected of having a neoplastic disorder, neoplasia, tumor, malignancy or cancer. In some embodiments a subject in need of a treatment, cell or composition described herein has or is suspected of having a neoplastic disorder, neoplasia, tumor, malignancy or cancer. In certain embodiments an engineered T cell described herein is used to treat a subject having, or suspected of having, a neoplastic disorder, neoplasia, tumor, malignancy or cancer.

In some embodiments, presented herein is a method of treating a subject having or suspected of having, a neoplasia, neoplastic disorder, tumor, cancer, or malignancy. In certain embodiments, a method of treating a subject comprises administering a therapeutically effective amount of an engineered T cell to a subject. In certain embodiments, a method comprises reducing or inhibiting proliferation of a neoplastic cell, tumor, cancer or malignant cell, comprising contacting the cell, tumor, cancer or malignant cell, with the engineered T cell in an amount sufficient to reduce or inhibit proliferation of the neoplastic cell, tumor, cancer or malignant cell.

In some embodiments, a method of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy, comprises administering to a subject an amount of an engineered T cell sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

Non-limiting examples of a neoplasia, neoplastic disorder, tumor, cancer or malignancy include a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. A neoplasia, neoplastic disorder, tumor, cancer or malignancy may comprise or involve hematopoietic cells. Non-limiting examples of a sarcoma include a lymphosarcoma, liposarcoma, osteosarcoma, chondrosarcoma, leiomyosarcoma, rhabdomyosarcoma or fibrosarcoma. In some embodiments, a neoplasia, neoplastic disorder, tumor, cancer or malignancy is a myeloma, lymphoma or leukemia. In some embodiments, a neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer. In some embodiments, a neoplasia, neoplastic disorder, tumor, cancer or malignancy comprises a small cell lung or non-small cell lung cancer. In some embodiments, a neoplasia, neoplastic disorder, tumor, cancer or malignancy comprises a stem cell neoplasia, tumor, cancer or malignancy. In some embodiments, a neoplasia, neoplastic disorder, tumor, cancer or malignancy.

In some embodiments, a method inhibits, or reduces relapse or progression of the neoplasia, neoplastic disorder, tumor, cancer or malignancy. In some embodiments, a method comprises administering an anti-cell proliferative, anti-neoplastic, anti-tumor, anti-cancer or immune-enhancing treatment or therapy. In some embodiments, a method of treatment results in partial or complete destruction of the neoplastic, tumor, cancer or malignant cell mass; a reduction in volume, size or numbers of cells of the neoplastic, tumor, cancer or malignant cell mass; stimulating, inducing or increasing neoplastic, tumor, cancer or malignant cell necrosis, lysis or apoptosis; reducing neoplasia, tumor, cancer or malignancy cell mass; inhibiting or preventing progression or an increase in neoplasia, tumor, cancer or malignancy volume, mass, size or cell numbers; or prolonging lifespan. In some embodiments, a method of treatment results in reducing or decreasing severity, duration or frequency of an adverse symptom or complication associated with or caused by the neoplasia, tumor, cancer or malignancy. In some embodiments, a method of treatment results in reducing or decreasing pain, discomfort, nausea, weakness or lethargy. In some embodiments, a method of treatment results in increased energy, appetite, improved mobility or psychological well-being.

The term “contacting” means direct or indirect binding or interaction between two or more entities (e.g., between target cell population and a T cell engineered to reduce or eliminate expression and/or function of a NR4A transcription factor in said cell). A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “binds” or “antibody binding” or “specific binding” means the contact between the antigen binding domain of an antibody, antibody fragment, CAR, TCR, engineered TCR, BCR, MHC, immunoglobulin-like molecule, scFv, CDR or other antigen presentation molecule and an antigen, epitope, or peptide with a binding affinity (K_{D}) of less than 10⁻⁵M. In some aspects, an antigen binding domain binds to both a complex of both an antigen and an MHC molecule. In some aspects, antigen binding domains bind with affinities of less than about 10⁻⁶ M, 10⁻⁷M, and preferably 10⁻⁸M, 10⁻⁹M, 10⁻¹⁰ M, 10⁻¹¹ M, or 10⁻¹² M. In a particular aspects, specific binding refers to the binding of an antigen to an MHC molecule, or the binding of an antigen binding domain of an engineered T-cell receptor to an antigen or antigen-MHC complex.

As used herein, the terms “exhausted T cells” or “exhausted CAR T cells” refer to the hyporesponsive⁷ (“exhausted”⁸⁻¹¹ or “dysfunctional”^{12,13}) state that is triggered by chronic antigen stimulation and characterized by upregulation of several inhibitory receptors and loss of effector function^{14,15}.

As used herein, the term “administer” and “administering” are used to mean introducing the therapeutic agent (e.g. polynucleotide, vector, cell, modified cell, population) into a subject. The therapeutic administration of this substance serves to attenuate any symptom, or prevent additional symptoms from arising. When administration is for the purposes of preventing or reducing the likelihood of developing an autoimmune disease or disorder, the substance is provided in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

As used herein, the term “expression” or “express” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound. The terms “upregulate” and “downregulate” and variations thereof when used in context of gene expression, respectively, refer to the increase and decrease of gene expression relative to a normal or expected threshold expression for cells, in general, or the sub-type of cell, in particular.

As used herein, the term “gene expression profile” refers to measuring the expression level of multiple genes to establish an expression profile for a particular sample.

As used herein, the term “reduce or eliminate expression and/or function of” refers to reducing or eliminating the transcription of said polynucleotides into mRNA, or alternatively reducing or eliminating the translation of said mRNA into peptides, polypeptides, or proteins, or reducing or eliminating the functioning of said peptides, polypeptides, or proteins. In a non-limiting example, the transcription of polynucleotides into mRNA is reduced to at least half of its normal level found in wild type cells.

As used herein, the term “increase expression of” refers to increasing the transcription of said polynucleotides into mRNA, or alternatively increasing the translation of said mRNA into peptides, polypeptides, or proteins, or increasing the functioning of said peptides, polypeptides, or proteins. In a non-limiting example, the transcription of polynucleotides into mRNA is increased to at least twice of its normal level found in wild type cells.

As used herein, the term “transcription factors” refers to polypeptides that are capable of sequence-specific interaction with a portion of a gene or gene regulatory region.

The interaction may be direct sequence-specific binding where the transcription factor directly contacts the nucleic acid or indirect sequence-specific binding mediated or facilitated by other auxiliary proteins where the transcription factor is tethered to the nucleic acid by a direct nucleic acid binding protein. In addition, some transcription factors demonstrate induced or synergistic binding. Transcription factors affect the level of gene transcription. In one aspect, transcription factors upregulate or increase gene expression. In another aspect, transcription factors downregulate or decrease gene expression.

As used herein, the term “NR4A transcription factor” refers to the members of the NR4A subfamily of nuclear hormone receptors that bind to DNA and modulate gene expression. Non-limiting examples of members of NR4A transcription factor family are human NR4A1 (Nur77), NR4A2 (Nurr1) and NR4A3 (NOR1) encoded by the sequences provided in SEQ ID NO:3, SEQ ID NO:4 and SEQ ID NO:5, respectively.

As used herein, the term “TOX transcription factor” refers to the members of the TOX subfamily of nuclear hormone receptors that bind to DNA and modulate gene expression. Non-limiting examples of members of TOX transcription factor family are human TOX1, TOX2, TOX3 and TOX4 encoded by the sequences provided in SEQ ID NO:6, SEQ ID NO:7, SEQ ID NO:8 and SEQ ID NO:9, respectively.

As used herein, the term “IL-21” (interleukin 21) refers to the members of the common-gamma chain family of cytokines with immunoregulatory activity. A non-limiting example is human IL-21 encoded by the sequence provided in SEQ ID NO:10.

As used herein, the term “inhibit expression and/or function of NFAT/AP-1 pathway” refers to reducing or eliminating the transcription of genes in the pathway, or alternatively reducing or eliminating the translation of said mRNA into pathway peptides, polypeptides, or proteins, or reducing or eliminating the functioning of said pathway peptides, polypeptides, or proteins. Non-limiting examples of inhibiting expression and/or function of NFAT/AP-1 pathway include inhibiting and/or function of NR4A transcription factor or TOX transcription factor, or alternatively increasing expression of IL-21.

An “an effective amount” or “efficacious amount” is an amount sufficient to achieve the intended purpose, non-limiting examples of such include: initiation of the immune response, modulation of the immune response, suppression of an inflammatory response and modulation of T cell activity or T cell populations. In one aspect, the effective amount is one that functions to achieve a stated therapeutic purpose, e.g., a therapeutically effective amount. As described herein in detail, the effective amount, or dosage, depends on the purpose and the composition, and can be determined according to the present disclosure.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “engineered T-cell receptor” refers to a molecule comprising the elements of (a) an extracellular antigen binding domain, (b) a transmembrane domain, and (c) an intracellular signaling domain. In some aspects, an engineered T-cell receptor is a genetically modified TCR, a modified TCR, a recombinant TCR, a transgenic TCR, a partial TCR, a chimeric fusion protein, a CAR, a first generation CAR, a second generation CAR, a third generation CAR, or a fourth generation TRUCK. In some aspects, the engineered T-cell receptor comprises an antibody or a fragment of an antibody. In particular aspects, the engineered T-cell receptor is a genetically modified TCR or a CAR.

As used herein, the term “receptor” or “T-cell receptor” or “TCR” refers to a cell surface molecule found on T-cells that functions to recognize and bind antigens presented by antigen presenting molecules. Generally, a TCR is a heterodimer of an alpha chain (TRA) and a beta chain (TRB). Some TCRs are comprised of alternative gamma (TRG) and delta (TRD) chains. T-cells expressing this version of a TCR are known as γδ T-cells. TCRs are part of the immunoglobulin superfamily. Accordingly, like an antibody, the TCR comprises three hypervariable CDR regions per chain. There is also an additional area of hypervariability on the beta-chain (HV4). The TCR heterodimer is generally present in an octomeric complex that further comprises three dimeric signaling modules CD3γ/ε, CD3δ/ε, and CD247 ζ/ζ or ζη. Non-limiting exemplary amino acid sequence of the human TCR-alpha chain: METLLGVSLVILWLQLARVNSQQGEEDPQALSIQEGENATMNCS YKTSINNLQWYRQNSGRGLVHLILIRSNEREKHSGRLRVTLDTSKKSSSLLITASRAA DTASYFCAPVLSGGGADGLTFGKGTHLIIQPYIQNPDPAVYQLRDSKSSDKSVCLFTD FDSQTNVSQSKDSDVYITDKTVLDMRSMDFKSNSAVAWSNKSDFACANAFNNSIIPE D TFFPSPESSCDVKLVEKSFETDTNLNFQNLSVIGFRILLLKVAGFNLLMTLRLWSS. Non-limiting exemplary amino acid sequence of the human TCR-beta chain: DSAVYLCASSLLRVYEQYFGPGTRLTVTEDLKNVFPPEVAVFEP PEAEISHTQKATLVCLATGFYPDHVELSWWVNGKEVHSGVSTDPQPLKEQP.

The term “modified TCR” refers to a TCR that has been genetically engineered, and/or a transgenic TCR, and/or a recombinant TCR. Non-limiting examples of modified TCRs include single-chain VαVβ TCRs (scTv), full-length TCRs produced through use of a T cell display system, and TCRs wherein the CDR regions have been engineered to recognize a specific antigen, peptide, fragment, and/or MHC molecule. Methods of developing and engineering modified TCRs are known in the art. For example, see Stone, J. D. et al. Methods in Enzymology 503: 189-222 (2012), PCT Application WO2014018863 A1.

Type-1 T Regulatory (T_{R}1) cells are a subset of CD4+ T cells that have regulatory properties and are able to suppress antigen-specific immune responses in vitro and in vivo. These T_{R}1 cells are defined by their unique profile of cytokine production and make high levels of IL-10 and TGF-beta, but no IL-4 or IL-2. The IL-10 and TGF-beta produced by these cells mediate the inhibition of primary naive T cells in vitro. There is also evidence that T_{R} cells exist in vivo, and the presence of high IL-10-producing CD4(+) T cells in patients with severe combined immunodeficiency who have received allogeneic stem-cell transplants have been documented. T_{R}1 cells are involved in the regulation of peripheral tolerance and they could potentially be used as a cellular therapy to modulate immune responses in vivo. See, for example, Levings, M. et al. J. Allergy Clin. Immunol. 106(1 Pt2):S109-12 (2000).

T_{R}1 cells are defined by their ability to produce high levels of IL-10 and TGF-beta. Tr1 cells specific for a variety of antigens arise in vivo, but may also differentiate from naive CD4+ T cells in the presence of IL-10 in vitro. T_{R}1 cells have a low proliferative capacity, which can be overcome by IL-15. T_{R}1 cells suppress naive and memory T helper type 1 or 2 responses via production of IL-10 and TGF-beta. Further characterization of T_{R}1 cells at the molecular level will define their mechanisms of action and clarify their relationship with other subsets of Tr cells. The use of T_{R}1 cells to identify novel targets for the development of new therapeutic agents, and as a cellular therapy to modulate peripheral tolerance, can be foreseen. See, for example, Roncarolo, M. et al. Immunol. Rev. 182:68-79 (2001).

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a human has or is suspected of having a cancer or neoplastic disorder.

A “composition” typically intends a combination of the active agent, e.g., an engineered immune cell, e.g. T-cell, a modified T-cell, a NK cell, a chimeric antigen cell, a cell comprising an engineered immune cell, e.g. a T-cell, a NK cell, a CAR T cell or a CAR NK cell, an antibody, a cytokine, IL-12, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

As used herein, the term “animal” refers to living multi-cellular vertebrate organisms, a category that includes, for example, mammals and birds. The term “mammal” includes both human and non-human mammals, e.g., bovines, canines, felines, rat, murines, simians, equines and humans. Additional examples include adults, juveniles and infants.

As used herein, the term “antibody” (“Ab”) collectively refers to immunoglobulins (or “Ig”) or immunoglobulin-like molecules including but not limited to antibodies of the following isotypes: IgM, IgA, IgD, IgE, IgG, and combinations thereof. Immunoglobulin-like molecules include but are not limited to similar molecules produced during an immune response in a vertebrate, for example, in mammals such as humans, rats, goats, rabbits and mice, as well as non-mammalian species, such as shark immunoglobulins (see Feige, M. et al. Proc. Nat. Ac. Sci. 41(22): 8155-60 (2014)). Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

As used herein, the term “monoclonal antibody” refers to an antibody produced by a cell into which the light and heavy chain genes of a single antibody have been transfected or, more traditionally, by a single clone of B-lymphocytes. Monoclonal antibodies generally have affinity for a single epitope (i.e. they are monovalent) but may be engineered to be specific for two or more epitopes (e.g. bispecific). Methods of producing monoclonal antibodies are known to those of skill in the art, for example by creating a hybridoma through fusion of myeloma cells with immune spleen cells, phage display, single cell amplification from B-cell populations, single plasma cell interrogation technologies, and single B-cell culture. Monoclonal antibodies include recombinant antibodies, chimeric antibodies, humanized antibodies, and human antibodies.

The general structure of an antibody is comprised of heavy (H) chains and light (L) chains connected by disulfide bonds. The structure can also comprise glycans attached at conserved amino acid residues. Each heavy and light chain contains a constant region and a variable region (also known as “domains”). There are two types of light chain, lambda (2) and kappa (κ). There are five primary types of heavy chains which determine the isotype (or class) of an antibody molecule: gamma (γ), delta (δ), alpha (α), mu (μ) and epsilon (ε). The constant regions of the heavy chain also contribute to the effector function of the antibody molecule. Antibodies comprising the heavy chains μ, δ, γ3, γ1, α1, γ2, γ4, ε, and α2 result in the following isotypes: IgM, IgD, IgG3, IgG1, IgA1, IgG2, IgG4, IgE, and IgA2, respectively. An IgY isotype, related to mammalian IgG, is found in reptiles and birds. An IgW isotype, related to mammalian IgD, is found in cartilaginous fish. Class switching is the process by which the constant region of an immunoglobulin heavy chain is replaced with a different immunoglobulin heavy chain through recombination of the heavy chain locus of a B-cell to produce an antibody of a different isotype. Antibodies may exist as monomers (e.g. IgG), dimers (e.g. IgA), tetramers (e.g. fish IgM), pentamers (e.g. mammalian IgM), and/or in complexes with other molecules. In some embodiments, antibodies can be bound to the surface of a cell or secreted by a cell.

The variable regions of the immunoglobulin heavy and the light chains specifically bind the antigen. The “framework” region is a portion of the Fab that acts as a scaffold for three hypervariable regions called “complementarity-determining regions” (CDRs). A set of CDRs is known as a paratope. The framework regions of different light or heavy chains are relatively conserved within a species. The combined framework region of an antibody (comprising regions from both light and heavy chains), largely adopts a β-sheet conformation and the CDRs form loops which connect, and in some cases form part of, the β-sheet structure. Thus, framework regions act to position the CDRs in correct orientation by inter-chain, non-covalent interactions. The framework region and CDRs for numerous antibodies have been defined and are available in a database maintained online (Kabat et al., Sequences of Proteins of Immunological Interest, U.S. Department of Health and Human Services, 1991).

The CDRs of the variable regions of heavy and light chains (V_{H} and V_{L}) are responsible for binding to an epitope of an antigen. A limited number of amino acid positions within the CDRs are directly involved in antigen binding. These positions within the CDRs are called specificity determining residues (SDRs). The CDRs of a heavy or light chain are numbered sequentially starting from the N-terminal end (i.e. CDR1, CDR2, and CDR3). For example, a V_{L} CDR3 is the middle CDR located in the variable domain of the light chain of an antibody. A V_{H} CDR1 is the first CDR in the variable domain of a heavy chain of an antibody. An antibody that binds a specific antigen will have specific V_{H} and V_{L}, region sequences, and thus specific CDR sequences. Antibodies with different specificities (i.e. different combining sites for different antigens) have different CDRs.

The term “humanized” when used in reference to an antibody, means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs. Such antibodies typically have reduced immunogenicity and therefore a longer half-life in humans as compared to the non-human parent antibody from which one or more CDRs were obtained or are based upon.

An “antigen-binding fragment” (Fab) refers to the regions of an antibody corresponding to two of the three fragments produced by papain digestion. The Fab fragment comprises the region that binds to an antigen and is composed of one variable region and one constant region from both a heavy chain and a light chain. An F(ab′)₂ fragment refers to a fragment of an antibody digested by pepsin or the enzyme IdeS (immunoglobulin degrading enzyme from S. pyogenes) comprising two Fab regions connected by disulfide bonds. A single chain variable fragment (“scFv”) refers to a fusion protein comprising at least one V_{H} and at least one V_{L} region connected by a linker of between 5 to 30 amino acids. Methods and techniques of developing scFv that bind to specific antigens are known in the art (see, e.g. Ahmad, Z. A. et al., Clinical and Developmental Immunology, 2012: 980250 (2012)).

As used herein, the term “antigen” refers to a compound, composition, or substance that may be specifically bound and/or recognized by the products of specific humoral or cellular immunity and antigen recognition molecules, including but not limited to an antibody molecule, single-chain variable fragment (scFv), cell surface immunoglobulin receptor, B-cell receptor (BCR), T-cell receptor (TCR), engineered TCR, modified TCR, or CAR. The term “epitope” refers to an antigen or a fragment, region, site, or domain of an antigen that is recognized by an antigen recognition molecule. Antigens can be any type of molecule including but not limited to peptides, proteins, lipids, phospholipids haptens, simple intermediary metabolites, sugars (e.g., monosaccharides or oligosaccharides), hormones, and macromolecules such as complex carbo-hydrates (e.g., polysaccharides). Some non-limiting examples of antigens include antigens involved in autoimmune disease (including autoantigens), allergy, and graft rejection, tumor antigens, toxins, and other miscellaneous antigens. Non-limiting examples of tumor antigens include mesothelin, ROR1 and EGFRvIII, ephrin type-A receptor 2 (EphA2), interleukin (IL)-13r alpha 2, an EGFR VIII, a PSMA, an EpCAM, a GD3, a fucosyl GM1, a PSCA, a PLAC1, a sarcoma breakpoint, a Wilms Tumor 1, a hematologic differentiation antigen, a surface glycoprotein, a gangliosides (GM2), a growth factor receptor, a stromal antigen, a vascular antigen, or a combination thereof. Antigens expressed by pathogens include, but are not limited to microbial antigens such as viral antigens, bacterial antigens, fungal antigens, protozoa, and other parasitic antigens.

As used herein, the term “target cell population” refers to a population of cells that present antigens, which can be targeted by engineered T cells. Non-limiting examples of target cell populations include tumor cells, cancer cells and pathogen infected cells. Non-limiting examples of pathogens include viral and bacterial pathogens.

As used herein, the term “antigen binding domain” refers to any protein or polypeptide domain that can specifically bind to an antigen target (including target complexes of antigens and MHC molecules).

As used herein, the term “autologous,” in reference to cells, tissue, and/or grafts refers to cells, tissue, and/or grafts that are isolated from and then and administered back into the same subject, patient, recipient, and/or host. “Allogeneic” refers to non-autologous cells, tissue, and/or grafts.

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4-5-6-7-8-9-10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-1. Non-limiting exemplary sources for such commercially available cell lines include the American Type Culture Collection, or ATCC, (www.atcc.org/) and the German Collection of Microorganisms and Cell Cultures (https://www.dsmz.de/).

As used herein, a “target cell” is any cell that expresses the antigen target to which the engineered T cells can bind.

As used herein, a “cancer” is a disease state characterized by the presence in a subject of cells demonstrating abnormal uncontrolled replication and may be used interchangeably with the term “tumor.” In some embodiments, the cancer is a leukemia or a lymphoma. “Cell associated with the cancer” refers to those subject cells that demonstrate abnormal uncontrolled replication. In certain embodiments, the cancer is acute myeloid leukemia or acute lymphoblastic leukemia. As used herein a “leukemia” is a cancer of the blood or bone marrow characterized by an abnormal increase of immature white blood cells. The specific condition of acute myeloid leukemia (AML)—also referred to as acute myelogenous leukemia or acute myeloblastic leukemia—is a cancer of the myeloid origin blood cells, characterized by the rapid growth of abnormal myeloid cells that accumulate in the bone marrow and interfere with the production of normal blood cells. The specific condition of acute lymphoblastic leukemia (ALL)—also referred to as acute lymphocytic leukemia or acute lymphoid leukemia—is a cancer of the white blood cells, characterized by the overproduction and accumulation of malignant, immature leukocytes (lymphoblasts) resulting a lack of normal, healthy blood cells. As used herein a “lymphoma” is a cancer of the blood characterized by the development of blood cell tumors and symptoms of enlarged lymph nodes, fever, drenching sweats, unintended weight loss, itching, and constantly feeling tired.

As used herein, “pathogen infected cell population” or “pathogen infected cells” refer to a population of cells or cells infected with pathogens. Examples of pathogenic infections include, but are not limited to, infection by bacteria such as group A Streptococcus, Mycobacterium tuberculosis, Shigella flexneri, Salmonella enterica, Listeria monocytogenes, Francisella tularensis, and infection by viruses such as herpes simplex virus.

One of skill in the art can monitor expression of the transcription factors using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

One of skill in the art can use methods such as RNA interference (RNAi), CRISPR, TALEN, ZFN or other methods that target specific sequences to reduce or eliminate expression and/or function of NR4A or TOX transcription factors. CRISPR, TALEN, ZFN or other genome editing tools can also be used to increase expression and/or function of IL-21.

As used herein, “RNAi” (RNA interference) refers to the method of reducing or eliminating gene expression in a cell by targeting specific mRNA sequences for degradation via introduction of short pieces of double stranded RNA (dsRNA) and small interfering RNA (such as siRNA, shRNA or miRNA etc.) (Agrawal, N. et al.; Microbiol Mol Biol Rev. 2003; 67:657-685, Arenz, C. et al.; Naturwissenschaften. 2003; 90:345-359, Hannon G J.; Nature. 2002; 418:244-251).

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. “Gene editing” refers to a type of genetic engineering in which the nucleotide sequence of a target polynucleotide is changed through introduction of deletions, insertions, single stranded or double stranded breaks, or base substitutions to the polynucleotide sequence. In some aspects, CRISPR-mediated gene editing utilizes the pathways of non-homologous end-joining (NHEJ) or homologous recombination to perform the edits. Gene regulation refers to increasing or decreasing the production of specific gene products such as protein or RNA.

The term “gRNA” or “guide RNA” as used herein refers to guide RNA sequences used to target specific polynucleotide sequences for gene editing employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83).

The term “Cas9” refers to a CRISPR associated endonuclease referred to by this name. Non-limiting exemplary Cas9s include Staphylococcus aureus Cas9, nuclease dead Cas9, and orthologs and biological equivalents each thereof. Orthologs include but are not limited to Streptococcus pyogenes Cas9 (“spCas9”), Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

As used herein, “TALEN” (transcription activator-like effector nucleases) refers to engineered nucleases that comprise a non-specific DNA-cleaving nuclease fused to a TALE DNA-binding domain, which can target DNA sequences and be used for genome editing. Boch (2011) Nature Biotech. 29: 135-6; and Boch et al. (2009) Science 326: 1509-12; Moscou et al. (2009) Science 326: 3501. TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence. To produce a TALEN, a TALE protein is fused to a nuclease (N), which is a wild-type or mutated Fokl endonuclease. Several mutations to Fokl have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Bio. 200: 96. The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity. Miller et al. (2011) Nature Biotech. 29: 143-8. TALENs specific to sequences in immune cells can be constructed using any method known in the art, including various schemes using modular components. Zhang et al. (2011) Nature Biotech. 29: 149-53; Geibler et al. (2011) PLoS ONE 6: e19509.

As used herein, “ZFN” (Zinc Finger Nuclease) refers to engineered nucleases that comprise a non-specific DNA-cleaving nuclease fused to a zinc finger DNA binding domain, which can target DNA sequences and be used for genome editing. Like a TALEN, a ZFN comprises a Fokl nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5. ZFNs specific to sequences in immune cells can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; Guo et al. (2010) J. Mol. Bioi. 400: 96; U.S. Patent Publication 201110158957; and U.S. Patent Publication 2012/0060230.

A “cytotoxic cell” intends a cell that is capable of killing other cells or microbes. Examples of cytotoxic cells include but are not limited to CD8+ T cells, natural-killer (NK) cells, NKT cells, and neutrophils, which cells are capable of mediating cytotoxicity responses.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, “disease-relevant antigen” or refers to an antigen, epitope, or fragment thereof involved in the disease process or mechanism. For example, an inflammation-relevant antigen is an antigen or fragment thereof that, when presented, produces an immune response. An inflammation-relevant antigen producing such an effect is selected to treat the inflammation. Similarly, an autoimmunity-related antigen is an antigen that is relevant to an autoimmune disease and would not be selected for the treatment of a disorder or disease other than autoimmunity, e.g., cancer. Non-limiting, exemplary disease-relevant antigens are disclosed herein and further, such antigens may be determined for a particular disease based on the epitope screening techniques, mechanisms, and methods described herein.

The term “encode” as it is applied to nucleic acid sequences refers to a polynucleotide which is said to “encode” an RNA or polypeptide if, in its native state or when manipulated by methods well known to those skilled in the art, the nucleic acid can be transcribed and/or translated to produce a functional RNA (e.g. miRNA, siRNA, RNAi, tRNA, rRNA, snRNA, etc), an mRNA, or a polypeptide and/or a fragment thereof. The antisense strand is the complement of such a nucleic acid, and the encoding sequence can be deduced therefrom.

As used herein, the term “enhancer,” as used herein, denotes regulatory sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

In the context of a nucleic acid or amino acid sequence, the term “chimeric” intends that the sequence contains is comprised of at least one substituent unit (e.g. fragment, region, portion, domain, polynucleotide, or polypeptide) that is derived from, obtained or isolated from, or based upon other distinct physical or chemical entities. For example, a chimera of two or more different proteins may comprise the sequence of a variable region domain from an antibody fused to the transmembrane domain of a cell signaling molecule. In some aspects, a chimera intends that the sequence is comprised of sequences from at least two distinct species.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains. Non-limiting exemplary polynucleotide sequences that encode for components of each domain are disclosed herein, e.g.:

Hinge domain: IgG1 heavy chain hinge polynucleotide sequence: CTCGAGCCCAAATCTTGTGACAAAACTCACACATGCCCACCGTGCCCG, and optionally an equivalent thereof.

Transmembrane domain: CD28 transmembrane region polynucleotide sequence: TTTTGGGTGCTGGTGGTGGTTGGTGGAGTCCTGGCTTGCTATAGCTTGCTAGTAA CAGTGGCCTTTATTATTTTCTGGGTG, and optionally an equivalent thereof.

Intracellular domain: 4-1BB co-stimulatory signaling region polynucleotide sequence: AAACGGGGCAGAAAGAAACTCCTGTATATATTCAAACAACCATTTATGAGACCA GTACAAACTACTCAAGAGGAAGATGGCTGTAGCTGCCGATTTCCAGAAGAAGAA GAAGGAGGATGTGAACTG, and optionally an equivalent thereof.

Intracellular domain: CD28 co-stimulatory signaling region polynucleotide sequence: AGGAGTAAGAGGAGCAGGCTCCTGCACAGTGACTACATGAACATGACTCCCCGC CGCCCCGGGCCCACCCGCAAGCATTACCAGCCCTATGCCCCACCACGCGACTTCG CAGCCTATCGCTCC, and optionally an equivalent thereof.

Intracellular domain: CD3 zeta signaling region polynucleotide sequence: AGAGTGAAGTTCAGCAGGAGCGCAGACGCCCCCGCGTACCAGCAGGGCCAGAA CCAGCTCTATAACGAGCTCAATCTAGGACGAAGAGAGGAGTACGATGTTTTGGA CAAGAGACGTGGCCGGGACCCTGAGATGGGGGGAAAGCCGAGAAGGAAGAACC CTCAGGAAGGCCTGTACAATGAACTGCAGAAAGATAAGATGGCGGAGGCCTACA GTGAGATTGGGATGAAAGGCGAGCGCCGGAGGGGCAAGGGGCACGATGGCCTT TACCAGGGTCTCAGTACAGCCACCAAGGACACCTACGACGCCCTTCACATGCAG GCCCTGCCCCCTCGCTAA, and optionally an equivalent thereof.

Non-limiting examples of CAR extracellular domains capable of binding to antigens are the anti-CD19 binding domain sequences that specifically bind CD19 antigen as disclosed in the US20140271635 application.

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non-limiting examples of such domains are provided herein.

As used herein, the term “CD8 α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177. The sequences associated with the CD8 α hinge domain are provided in Pinto, R. D. et al. (2006) Vet. Immunol. Immunopathol. 110:169-177. Non-limiting examples of such include:

Human CD8 alpha hinge domain amino acid sequence: PAKPTTTPAPRPPTPAPTIASQPLSLRPEACRPAAGGAVHTRGLDFACDIY, and optionally an equivalent thereof.

Mouse CD8 alpha hinge domain amino acid sequence: KVNSTTTKPVLRTPSPVHPTGTSQPQRPEDCRPRGSVKGTGLDFACDIY, and optionally an equivalent thereof.

Cat CD8 alpha hinge domain amino acid sequence: PVKPTTTPAPRPPTQAPITTSQRVSLRPGTCQPSAGSTVEASGLDLSCDIY, and optionally an equivalent thereof.

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (GenBank Accession No: NP_001759.3), or the amino acid positions 197 to 217 of the mouse T-cell surface glycoprotein CD8 alpha chain (GenBank Accession No: NP_001074579.1), and the amino acid positions 190 to 210 of the rat T-cell surface glycoprotein CD8 alpha chain (GenBank Accession No: NP_113726.1) provide additional example sequences of the CD8 α transmembrane domain. The sequences associated with each of the listed accession numbers are provided as follows:

Human CD8 alpha transmembrane domain amino acid sequence: IYIWAPLAGTCGVLLLSLVIT, and optionally an equivalent thereof.

Mouse CD8 alpha transmembrane domain amino acid sequence: IWAPLAGICVALLLSLIITLI, and optionally an equivalent thereof.

Rat CD8 alpha transmembrane domain amino acid sequence: IWAPLAGICAVLLLSLVITLI, and optionally an equivalent thereof.

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. Non-limiting example sequences of the 4-1BB costimulatory signaling region are provided in U.S. Publication 20130266551A1 (filed as U.S. application Ser. No. 13/826,258), such as the exemplary sequence provided below and the sequence encoded by 4-1BB costimulatory signaling region amino acid sequence: KRGRKKLLYIFKQPFMRPVQTTQEEDGCSCRFPEEEEGGCEL, and optionally an equivalent thereof.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Patent Application Publication No. 2015/0017141A1 the exemplary polynucleotide sequence provided below.

ICOS costimulatory signaling region polynucleotide sequence: ACAAAAAAGA AGTATTCATC CAGTGTGCAC GACCCTAACG GTGAATACAT GTTCATGAGA GCAGTGAACA CAGCCAAAAA ATCCAGACTC ACAGATGTGA CCCTA, and optionally an equivalent thereof.

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Patent Application Publication No. 2012/20148552A1, and include the exemplary sequence provided below.

OX40 costimulatory signaling region polynucleotide sequence: AGGGACCAG AGGCTGCCCC CCGATGCCCA CAAGCCCCCT GGGGGAGGCA GTTTCCGGAC CCCCATCCAA GAGGAGCAGG CCGACGCCCA CTCCACCCTG GCCAAGATC, and optionally an equivalent thereof.

As used herein, the term “CD28 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD28 costimulatory signaling region sequence shown herein. The example sequences CD28 costimulatory signaling domain are provided in U.S. Pat. No. 5,686,281; Geiger, T. L. et al. (2001) Blood 98: 2364-2371; Hombach, A. et al. (2001) J Immunol 167: 6123-6131; Maher, J. et al. (2002) Nat Biotechnol 20: 70-75; Haynes, N. M. et al. (2002) J Immunol. 169: 5780-5786 (2002); Haynes, N. M. et al. (2002) Blood 100: 3155-3163. A non-limiting example include the sequence encoded by: CD28 amino acid sequence: MLRLLLALNL FPSIQVTGNK ILVKQSPMLV AYDNAVNLSC KYSYNLFSRE FRASLHKGLDSAVEVCVVYG NYSQQLQVYS KTGFNCDGKL GNESVTFYLQ NLYVNQTDIY FCKIEVMYPPPYLDNEKSNG TIIHVKGKHL CPSPLFPGPS KPFWVLVVVG GVLACYSLLVTVAFIIFWVR SKRSRLLHSD YMNMTPRRPG PTRKHYQPYA PPRDFAAYRS, and equivalents thereof.

As used herein, the term “CD3 zeta signaling domain” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD3 zeta signaling domain sequence as shown herein. Non-limiting example sequences of the CD3 zeta signaling domain amino acid sequence are provided in U.S. application Ser. No. 13/826,258, e.g.: RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR.

As used herein, a “first generation CAR” refers to a CAR comprising an extracellular domain capable of binding to an antigen, a transmembrane domain derived from a polypeptide different from a polypeptide from which the extracellular domain is derived, and at least one intracellular domain. A “second generation CAR” refers to a first generation CAR further comprising one costimulation domain (e.g. 4-1BB or CD28). A “third generation CAR” refers to a first generation CAR further comprising two costimulation domains (e.g. CD27, CD28, ICOS, 4-1BB, or OX40). A “fourth generation CAR” (also known as a “TRUCK”) refers to a CAR T-cell further engineered to secrete an additional factor (e.g. proinflammatory cytokine IL-12). A review of these CAR technologies and cell therapy is found in Maus, M. et al. Clin. Cancer Res. 22(3): 1875-84 (2016).

As used herein, the term “signal peptide” or “signal polypeptide” intends an amino acid sequence usually present at the N-terminal end of newly synthesized secretory or membrane polypeptides or proteins. It acts to direct the polypeptide across or into a cell membrane and is then subsequently removed. Examples of such are well known in the art. Non-limiting examples are those described in U.S. Pat. Nos. 8,853,381 and 5,958,736.

As used herein in reference to a regulatory polynucleotide, the term “operatively linked” refers to an association between the regulatory polynucleotide and the polynucleotide sequence to which it is linked such that, when a specific protein binds to the regulatory polynucleotide, the linked polynucleotide is transcribed.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “suicide gene” is a gene capable of inducing cell apoptosis; non-limiting examples include HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). Suicide genes may function along a variety of pathways, and, in some cases, may be inducible by an inducing agent such as a small molecule. For example, the iCasp suicide gene comprises portion of a caspase protein operatively linked to a protein optimized to bind to an inducing agent; introduction of the inducing agent into a cell comprising the suicide gene results in the activation of caspase and the subsequent apoptosis of said cell.

The term “transduce” or “transduction” as it is applied to the production of chimeric antigen receptor cells refers to the process whereby a foreign nucleotide sequence is introduced into a cell. In some embodiments, this transduction is done via a vector.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a gene of interest such as a polynucleotide encoding a CAR. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif.) and Promega Biotech (Madison, Wis.).

As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P, wherein X refers to any amino acid generally thought to be self-cleaving.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I. In one aspect, a detectable marker excludes naturally fluorescent polynucleotides.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A “normal cell corresponding to the cancer tissue type” refers to a normal cell from a same tissue type as the cancer tissue. A non-limiting example is a normal leukocyte from a patient, e.g. a patient with leukemia.

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “specific binding” means the contact between an antibody and an antigen with a binding affinity of at least 10⁻⁶ M. In certain aspects, antibodies bind with affinities of at least about 10⁻⁷ M, and preferably 10⁻⁸ M, 10⁻⁹ M, 10⁻¹⁰ M, 10⁻¹¹ M, or 10⁻¹² M.

A “solid tumor” is an abnormal mass of tissue that usually does not contain cysts or liquid areas. Solid tumors can be benign or malignant, metastatic or non-metastatic. Different types of solid tumors are named for the type of cells that form them. Examples of solid tumors include sarcomas, carcinomas, and lymphomas.

The sequences associated with each of the above listed GenBank Accession Nos., UniProt Reference Nos., and references are herein incorporated by reference.

As used herein, the term “major histocompatibility complex” (MHC) refers to an antigen presentation molecule that functions as part of the immune system to bind antigens and other peptide fragments and display them on the cell surface for recognition by antigen recognition molecules such as TCR. MHC may be used interchangeably with the term “human leukocyte antigen” (HLA) when used in reference to human MHC; thus, MHC refers to all HLA subtypes including, but not limited to, the classical MHC genes disclosed herein: HLA-A, HLA-B, HLA-C, HLA-E, HLA-F, HLA-G, HLA-DM, HLA-DO, HLA-DP, HLA-DQ, and HLA-DR, in addition to all variants, isoforms, isotypes, and other biological equivalents thereof. MHC class I (MHC-I) and MHC class II (MHC-II) molecules utilize distinct antigen processing pathways. In general, peptides derived from intracellular antigens are presented to CD8+ T cells by MHC class I molecules, which are expressed on virtually all cells, while extracellular antigen-derived peptides are presented to CD4+ T cells by MHC-II molecules. However, several exceptions to this dichotomy have been observed. In certain embodiments disclosed herein, a particular antigen, peptide, and/or epitope is identified and presented in an antigen-MHC complex in the context of an appropriate MHC class I or II protein. The genetic makeup of a subject may be assessed to determine which MHC allele is suitable for a particular patient, disease, or condition with a particular set of antigens. In mice, the MHC genes are known as the histocompatibility 2 (H-2) genes. Murine classical MHC class I subtypes include H-2D, H-2K, and H-2L. Murine non-classical MHC class I subtypes include H-2Q, H-2M, and H-2T. Murine classical MHC class II subtypes include H-2A (I-A), and H-2E (1-E). Non-classical murine MHC class II subtypes include H-2M and H-20. Canine MHC molecules are known as Dog Leukocyte Antigens (DLA). Feline MHC molecules are known as Feline Leukocyte Antigens (FLA). In some embodiments, an orthologous or homologous MHC molecule is selected to transition a therapy or treatment involving a specific antigen-MHC complex from one species to a different species.

As used herein, the phrase “immune response” or its equivalent “immunological response” refers to the development of a cell-mediated response (e.g. mediated by antigen-specific T cells or their secretion products). A cellular immune response is elicited by the presentation of polypeptide epitopes in association with Class I or Class II MHC molecules, to treat or prevent a viral infection, expand antigen-specific Breg cells, TC1, CD4+T helper cells and/or CD8+ cytotoxic T cells and/or disease generated, autoregulatory T cell and B cell “memory” cells. The response may also involve activation of other components. In some aspects, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells—non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

“Immune cells” include all cells that are produced by hematopoietic stem cells (HSC) including, but not limited to, HSCs, white blood cells (leukocytes), lymphocytes (including T cells, B cells, and natural killer (NK) cells) and myeloid-derived cells (neutrophils, eosinophils, basophils, monocytes, macrophages, dendritic cells). “Leukocytes” include but are not limited to lymphocytes, granulocytes, monocytes, and macrophages.

The terms “inflammatory response” and “inflammation” as used herein indicate the complex biological response of immune cells, humoral factors, and vascular tissues of an individual or subject to exogenous or endogenous stimuli, such as pathogens, damaged cells, or irritants, and/or inflammatory signals such as pro-inflammatory cytokines. The inflammatory response includes secretion of cytokines and, more particularly, of pro-inflammatory cytokines, i.e. cytokines which are produced predominantly by activated immune cells and are involved in the amplification of inflammatory reactions. Exemplary pro-inflammatory cytokines and chemokines include but are not limited to IL-1β, TNF-α, IFN-γ, IL-8, IL-6, IL-12, IL-15, IL-16, IL-17 (including family members IL17A, IL17B, IL-17C, IL-17D, IL-17E, IL-17F), IL-18, GM-CSF, IL-21, IL-23, IL-27 and TGF-β. Exemplary anti-inflammatory cytokines include but are not limited to TGF-β, IL-1Rα, IL-4, IL-6, IL-10, IL-11, IL-13, IL-35, INF-α. A cytokine may have either pro-inflammatory and anti-inflammatory properties depending on the particular biological context (Cavaillon, J. M (2001) Cell Mol. Biol 47(4): 695-702). Exemplary inflammations include acute inflammation and chronic inflammation. Acute inflammation indicates a short-term process characterized by the classic signs of inflammation (swelling, redness, pain, heat, and loss of function) due to the infiltration of the tissues by plasma and leukocytes. An acute inflammation typically occurs as long as the injurious stimulus is present and ceases once the stimulus has been removed, broken down, or walled off by scarring (fibrosis). Chronic inflammation indicates a condition characterized by concurrent active inflammation, tissue destruction, and attempts at repair. Chronic inflammation is not characterized by the classic signs of acute inflammation listed above. Instead, chronically inflamed tissue is characterized by the infiltration of mononuclear immune cells (monocytes, macrophages, lymphocytes, and plasma cells), tissue destruction, and attempts at healing, which include angiogenesis and fibrosis. An inflammation can be inhibited in the sense of the present disclosure by affecting and in particular inhibiting any one of the events that form the complex biological response associated with an inflammation in an individual.

“Autoimmune disease or disorder” includes diseases or disorders arising from and directed against an individual's own tissues or organs or manifestation thereof or a condition resulting there from. In one embodiment, it refers to a condition that results from, or is aggravated by, the production by T cells that are reactive with normal body tissues and antigens. Examples of autoimmune diseases or disorders include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica spectrum disorder (NMO, also known as Devic's Disease or Devic's Syndrome), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, non-granulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, Type I diabetes, Type II diabetes, latent autoimmune diabetes in adults (or Type 1.5 diabetes) Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and giant T cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, anti-phospholipid syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, acquired thrombocytopenic purpura, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Graves disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), autoimmune ear disease such as autoimmune inner ear disease (AIED), autoimmune hearing loss, polychondritis such as refractory or relapsed or relapsing polychondritis, pulmonary alveolar proteinosis, Cogan's syndrome/non-syphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or non-purulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, hyperalgesia, renal ischemia, cerebral ischemia, and disease accompanying vascularization, allergic hypersensitivity disorders, glomerulonephritides, reperfusion injury, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, emphysema, alopecia areata, adipose tissue inflammation/diabetes type II, obesity associated adipose tissue inflammation/insulin resistance, and endometriosis.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g. a viral vector). In some embodiments, transfection is done via a chemical carrier, DNA/liposome complex, or micelle (e.g. Lipofectamine (Invitrogen)). In some embodiments, viral infection is done via infecting the cells with a viral particle comprising the polynucleotide of interest (e.g. AAV). In some embodiments, introduction further comprises CRISPR mediated gene editing or Transcription activator-like effector nuclease (TALEN) mediated gene editing. Methods of introducing non-nucleic acid foreign agents (e.g. soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing T cells in a culture medium under conditions that favor expansion and proliferation of the cell. The term “culture medium” or “medium” is recognized in the art, and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

“Cryoprotectants” are known in the art and include without limitation, e.g., sucrose, trehalose, and glycerol. A cryoprotectant exhibiting low toxicity in biological systems is generally used.

### Modes for Carrying Out the Disclosure

To identify transcriptional and other regulators contributing to the diminished function of CAR T cells in solid tumors, a mouse model was developed in which recipient mice bearing murine melanoma tumors expressing huCD19 antigen were adoptively transferred with huCD19-reactive CART cells. CD8⁺ CAR-expressing tumor-infiltrating T cells (CAR TILs) and endogenous TILs with low effector function and high expression of inhibitory surface receptors PD-1 and TIM3 exhibit similar profiles of gene expression and chromatin accessibility, associated with secondary activation of the three Nr4a (Nuclear Receptor Subfamily 4 Group A) transcription factors Nr4a1 (Nur77), Nr4a2 (Nurr1) and Nr4a3 (Nor1) by the initiating transcription factor NFAT (nuclear factor of activated T cells)¹⁶⁻¹⁸. Through a similar comparison of data from human CD8⁺ TILs^{19,20} and viral antigen specific CD8⁺ T cells from humans with chronic infections²¹, and observed high expression of Nr4a transcription factors and enrichment of Nr4a binding motifs in uniquely accessible chromatin regions. Treatment of tumor-bearing mice with CART cells lacking all three Nr4a transcription factors (Nr4a TKO) resulted in tumor regression and prolonged survival compared to mice adoptively transferred with Nr4a-sufficient (WT) CAR T cells. Nr4a TKO CAR TILs displayed phenotypes and gene expression profiles characteristic of CD8⁺ effector T cells, and chromatin regions uniquely accessible in Nr4a TKO CAR TILs compared to wild type were enriched for binding motifs for transcription factors classically involved in T cell activation. As described herein, Nr4a transcription factors were identified as major players in the cell-intrinsic program of T cell hyporesponsiveness and point to Nr4a inhibition as a promising strategy for cancer immunotherapy.

### Engineered Immune Cells

To that end, provided herein is a cell engineered to reduce or eliminate expression and/or function of an NR4A transcription factor in the cell. In one aspect, the cell is engineered to reduce or eliminate expression and/or function of an NR4A transcription factor in the cell, wherein the NR4A transcription factor comprises, or alternatively consists essentially of, or yet further consists of one, two or all three of NR4A1 (Nur77), NR4A2 (Nurr1) or NR4A3 (NOR′). Expression can be reduced by at least 10% or more, or 20% or 30%, or 40%, or 50%, or 60%, or 70%, or 80%, or 85%, or 90%, or 95%, or 99% or 100% as compared to a comparative wild-type cell. Non-limiting examples of cells are immune cells, such as for example T cells and NK cells.

In another aspect, a cell is engineered to reduce or eliminate expression and/or function of an NR4A transcription factor in the cell comprises, or alternatively consists essentially of, or yet further consists of the gene expression profile as shown in Table 1 and/or Table 2. Expression can be reduced by at least 10% or more, or 20% or 30%, or 40%, or 50%, or 60%, or 70%, or 80%, or 85%, or 90%, or 95%, or 99% or 100% as compared to a comparative wild-type cell. Non-limiting examples of cells are immune cells, such as for example T cells and NK cells.

Also provided herein is a cell engineered to reduce or eliminate expression and/or function of a TOX transcription factor in the cell. In one aspect, the cell is engineered to reduce or eliminate expression and/or function of a TOX transcription factor, wherein the TOX transcription factor comprises, or alternatively consists essentially of, or yet further consists of TOX1, TOX2, TOX3 or TOX4. In another aspect, the cell is engineered to reduce or eliminate expression and/or function of two or more of TOX1, TOX2, TOX3 or TOX4. In a further aspect, the cell is engineered to reduce or eliminate expression and/or function of four of TOX1, TOX2, TOX3 or TOX4. Expression can be reduced by at least 10% or more, or 20% or 30%, or 40%, or 50%, or 60%, or 70%, or 80%, or 85%, or 90%, or 95%, or 99% or 100% as compared to a comparative wild-type cell. Non-limiting examples of cells are immune cells, such as for example T cells and NK cells.

Also provided herein is an immune cell engineered to reduce or eliminate expression and/or function of an NR4A and a TOX transcription factor in said immune cell. In one aspect, the immune cell is engineered to reduce or eliminate expression and/or function of an NR4A and a TOX transcription factor in said immune cell, wherein the NR4A transcription factor comprises, or alternatively consists essentially of, or yet further consists of NR4A1 (Nur77), NR4A2 (Nurr1) or NR4A3 (NOR1) and wherein the TOX transcription factor comprises, or alternatively consists essentially of, or yet further consists of TOX1, TOX2, TOX3 or TOX4. In another aspect, wherein the immune cell is engineered to reduce or eliminate expression and/or function of two or more, or three or more of NR4A1 (Nur77), NR4A2 (Nurr1) and NR4A3 (NOR1). Alternatively, the cell can be engineered to reduce one, two, three or all four TOX factors, and one, two, three of all 4 NR4A transcription factors. Expression can be reduced by at least 10% or more, or 20% or 30%, or 40%, or 50%, or 60%, or 70%, or 80%, or 85%, or 90%, or 95%, or 99% or 100% as compared to a comparative wild-type cell.

Further provided herein is an immune cell engineered to reduce or eliminate expression and/or function of an NR4A and a TOX transcription factor, as described above and incorporated by reference herein, and increase expression of IL-21 in said T cell. As used herein, the term “IL-21” (interleukin 21) refers to the members of the common-gamma chain family of cytokines with immunoregulatory activity. A non-limiting example is human IL-21 encoded by the sequence provided in SEQ ID NO:10. Increased expression includes, for example at least about 2%, or alternatively about 5%, or alternatively at least 10%, or alternatively at least 15%, or alternatively at least 20%, alternatively at least 100% or more, or 150% or 200%, or 250%, or 300%, or 350%, or 400%, or 450%, or 500%, or 550%, or 600%, or 650% or 700% or more as compared to a comparative wild-type cell.

Also provided herein is an immune engineered to inhibit expression and/or function of NFAT/AP-1 pathway in said immune cell. Expression can be reduced by at least 10% or more, or 20% or 30%, or 40%, or 50%, or 60%, or 70%, or 80%, or 85%, or 90%, or 95%, or 99% or 100% as compared to a comparative wild-type cell.

As used herein, the term “inhibit expression and/or function of NFAT/AP-1 pathway” refers to reducing or eliminating the transcription of genes in the pathway, or alternatively reducing or eliminating the translation of said mRNA into pathway peptides, polypeptides, or proteins, or reducing or eliminating the functioning of said pathway peptides, polypeptides, or proteins. Non-limiting examples of inhibiting expression and/or function of NFAT/AP-1 pathway include inhibiting and/or function of NR4A transcription factor and/or TOX transcription factor, or alternatively increasing expression of IL-21. Increased expression includes, for example at least about 2%, or alternatively about 5%, or alternatively at least 10%, or alternatively at least 15%, or alternatively at least 20%, alternatively at least 100% or more, or 150% or 200%, or 250%, or 300%, or 350%, or 400%, or 450%, or 500%, or 550%, or 600%, or 650% or 700% or more as compared to a comparative wild-type cell.

One of skill in the art can monitor expression of the transcription factors using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

One of skill in the art can use methods such as RNA interference (RNAi), CRISPR, TALEN, ZFN or other methods that target specific sequences to reduce or eliminate expression and/or function of NR4A or TOX transcription factors. CRISPR, TALEN, ZFN or other genome editing tools can also be used to increase expression and/or function of IL-21.

The cells can be isolated from a host or cultured immune cells. Non-limiting samples of such include mammalian, and human cells, as defined herein. In one aspect the immune cells are NK cells or T cells. When used for treatment, they can be autologous or allogeneic to the subject being treated. “T cell” for the purpose of this disclosure include all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/). The cells can be from any multi-cellular vertebrate organism such s for example, mammals and birds. The term “mammal” includes both human and non-human mammals, e.g., bovines, canines, felines, rat, murines, simians, equines and humans. Additional examples include adults, juveniles and infants.

In some embodiments, the engineered immune cell of this disclosure is a CD8 T cell. In other embodiments, the engineered immune cell of this disclosure is a CD3 cell, T-helper cell (CD4+ cell), natural killer (NK) T-cell, T-regulatory cell (Treg), gamma-delta T cell or a neutrophil.

In some embodiments, the engineered immune cell described above expresses a receptor that binds a tumor antigen or antigens expressed by pathogens. In further embodiments, the engineered T cell described above expresses a receptor that binds a tumor antigen, wherein the tumor antigen comprises, or alternatively consists essentially of, or yet further consists of mesothelin, ROR1, or EGFRvIII, ephrin type-A receptor 2 (EphA2), interleukin (IL)-13r alpha 2, an EGFR VIII, a PSMA, an EpCAM, a GD3, a fucosyl GM1, a PSCA, a PLAC1, a sarcoma breakpoint, a Wilms Tumor 1, a hematologic differentiation antigen, a surface glycoprotein, a gangliosides (GM2), a growth factor receptor, a stromal antigen, a vascular antigen, or a combination thereof.

In a particular embodiment, the engineered immune cell of this disclosure wherein further comprises, or alternatively consists essentially of, or yet further consists of a suicide gene. As used herein, the term “suicide gene” is a gene capable of inducing cell apoptosis; non-limiting examples include HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). Suicide genes may function along a variety of pathways, and, in some cases, may be inducible by an inducing agent such as a small molecule.

In one aspect the engineered immune cell of this disclosure comprises, or alternatively consists essentially of, or yet further consists of a chimeric antigen receptor (CAR) and thus, the engineered cell is a CAR cell. Thus, the engineered immune cell of this disclosure further comprises, or alternatively consists essentially of, or yet further consists of a chimeric antigen receptor (CAR), where the CAR further comprises, or alternatively consists essentially of, or yet further consists of: (a) an antigen binding domain; (b) a hinge domain; (c) a transmembrane domain; (d) and an intracellular domain.

In one aspect, the engineered immune cell of this disclosure comprises, or alternatively consists essentially of, or yet further consists of a chimeric antigen receptor (CAR), wherein the chimeric antigen receptor (CAR) comprises, or alternatively consists essentially of, or yet further consists of: (a) an anti-CD19 binding domain; (b) a hinge domain; (c) a CD28 or a CD8 α transmembrane domain; (d) one or more costimulatory regions selected from a CD28 costimulatory signaling region, a 4-1BB costimulatory signaling region, an ICOS costimulatory signaling region, and an OX40 costimulatory region; and (e) a CD3 zeta signaling domain. The following sequences are merely exemplary for use in making the cells as described herein:

Hinge domain: IgG1 heavy chain hinge polynucleotide sequence: CTCGAGCCCAAATCTTGTGACAAAACTCACACATGCCCACCGTGCCCG, and optionally an equivalent thereof.

Transmembrane domain: CD28 transmembrane region polynucleotide sequence: TTTTGGGTGCTGGTGGTGGTTGGTGGAGTCCTGGCTTGCTATAGCTTGCTAGTAA CAGTGGCCTTTATTATTTTCTGGGTG, and optionally an equivalent thereof.

Intracellular domain: 4-1BB co-stimulatory signaling region polynucleotide sequence: AAACGGGGCAGAAAGAAACTCCTGTATATATTCAAACAACCATTTATGAGACCA GTACAAACTACTCAAGAGGAAGATGGCTGTAGCTGCCGATTTCCAGAAGAAGAA GAAGGAGGATGTGAACTG, and optionally an equivalent thereof.

Intracellular domain: CD28 co-stimulatory signaling region polynucleotide sequence: AGGAGTAAGAGGAGCAGGCTCCTGCACAGTGACTACATGAACATGACTCCCCGC CGCCCCGGGCCCACCCGCAAGCATTACCAGCCCTATGCCCCACCACGCGACTTCG CAGCCTATCGCTCC, and optionally an equivalent thereof.

Intracellular domain: CD3 zeta signaling region polynucleotide sequence: AGAGTGAAGTTCAGCAGGAGCGCAGACGCCCCCGCGTACCAGCAGGGCCAGAA CCAGCTCTATAACGAGCTCAATCTAGGACGAAGAGAGGAGTACGATGTTTTGGA CAAGAGACGTGGCCGGGACCCTGAGATGGGGGGAAAGCCGAGAAGGAAGAACC CTCAGGAAGGCCTGTACAATGAACTGCAGAAAGATAAGATGGCGGAGGCCTACA GTGAGATTGGGATGAAAGGCGAGCGCCGGAGGGGCAAGGGGCACGATGGCCTT TACCAGGGTCTCAGTACAGCCACCAAGGACACCTACGACGCCCTTCACATGCAG GCCCTGCCCCCTCGCTAA, and optionally an equivalent thereof.

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non-limiting examples of such domains are provided herein.

Non-limiting examples of CAR extracellular domains capable of binding to antigens are the anti-CD19 binding domain sequences that specifically bind CD19 antigen as disclosed in the US20140271635 application. Thus, the polynucleotide will encode this binding domain.

As used herein, the term “CD8 α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177. The sequences associated with the CD8 α hinge domain are provided in Pinto, R. D. et al. (2006) Vet. Immunol. Immunopathol. 110:169-177. Non-limiting examples of such include:

Human CD8 alpha hinge domain amino acid sequence: PAKPTTTPAPRPPTPAPTIASQPLSLRPEACRPAAGGAVHTRGLDFACDIY, and optionally an equivalent thereof.

Mouse CD8 alpha hinge domain amino acid sequence: KVNSTTTKPVLRTPSPVHPTGTSQPQRPEDCRPRGSVKGTGLDFACDIY, and optionally an equivalent thereof.

Cat CD8 alpha hinge domain amino acid sequence: PVKPTTTPAPRPPTQAPITTSQRVSLRPGTCQPSAGSTVEASGLDLSCDIY, and optionally an equivalent thereof.

Thus, polynucleotides encoding these peptides are comprised within the CAR-encoding polynucleotide.

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (GenBank Accession No: NP_001759.3), or the amino acid positions 197 to 217 of the mouse T-cell surface glycoprotein CD8 alpha chain (GenBank Accession No: NP_001074579.1), and the amino acid positions 190 to 210 of the rat T-cell surface glycoprotein CD8 alpha chain (GenBank Accession No: NP_113726.1) provide additional example sequences of the CD8 α transmembrane domain. The sequences associated with each of the listed accession numbers are provided as follows:

Human CD8 alpha transmembrane domain amino acid sequence: IYIWAPLAGTCGVLLLSLVIT, and optionally an equivalent thereof.

Mouse CD8 alpha transmembrane domain amino acid sequence: IWAPLAGICVALLLSLIITLI, and optionally an equivalent thereof.

Rat CD8 alpha transmembrane domain amino acid sequence: IWAPLAGICAVLLLSLVITLI, and optionally an equivalent thereof.

Thus, polynucleotides encoding these peptides are comprised within the polypeptide.

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. Non-limiting example sequences of the 4-1BB costimulatory signaling region are provided in U.S. Publication 20130266551A1 (filed as U.S. application Ser. No. 13/826,258), such as the exemplary sequence provided below.

4-1BB costimulatory signaling region amino acid sequence: KRGRKKLLYIFKQPFMRPVQTTQEEDGCSCRFPEEEEGGCEL, and optionally an equivalent thereof. Thus, a polynucleotide encoding this sequence is encoded within the polynucleotide.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Patent Application Publication No. 2015/0017141A1 the exemplary polynucleotide sequence provided below.

ICOS costimulatory signaling region polynucleotide sequence: ACAAAAAAGA AGTATTCATC CAGTGTGCAC GACCCTAACG GTGAATACAT GTTCATGAGA GCAGTGAACA CAGCCAAAAA ATCCAGACTC ACAGATGTGA CCCTA, and optionally an equivalent thereof.

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Patent Application Publication No. 2012/20148552A1, and include the exemplary sequence provided below.

OX40 costimulatory signaling region polynucleotide sequence: AGGGACCAG AGGCTGCCCC CCGATGCCCA CAAGCCCCCT GGGGGAGGCA GTTTCCGGAC CCCCATCCAA GAGGAGCAGG CCGACGCCCA CTCCACCCTG GCCAAGATC, and optionally an equivalent thereof.

As used herein, the term “CD28 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD28 costimulatory signaling region sequence shown herein. The example sequences CD28 costimulatory signaling domain are provided in U.S. Pat. No. 5,686,281; Geiger, T. L. et al. (2001) Blood 98: 2364-2371; Hombach, A. et al. (2001) J Immunol 167: 6123-6131; Maher, J. et al. (2002) Nat Biotechnol 20: 70-75; Haynes, N. M. et al. (2002) J Immunol 169: 5780-5786 (2002); Haynes, N. M. et al. (2002) Blood 100: 3155-3163. Non-limiting examples include the sequence below: CD28 amino acid sequence: MLRLLLALNL FPSIQVTGNK ILVKQSPMLV AYDNAVNLSC KYSYNLFSRE FRASLHKGLDSAVEVCVVYG NYSQQLQVYS KTGFNCDGKL GNESVTFYLQ NLYVNQTDIY FCKIEVMYPPPYLDNEKSNG TIIHVKGKHL CPSPLFPGPS KPFWVLVVVG GVLACYSLLVTVAFIIFWVR SKRSRLLHSD YMNMTPRRPG PTRKHYQPYA PPRDFAAYRS, and equivalents thereof.

Thus, polynucleotides encoding these polypeptides are included within the CAR encoding polypeptide.

As used herein, the term “CD3 zeta signaling domain” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD3 zeta signaling domain sequence as shown herein. Non-limiting example sequences of the CD3 zeta signaling domain amino acid sequence are provided in U.S. application Ser. No. 13/826,258, e.g.: RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQAL PPR. Thus, a polynucleotide encoding these polypeptides are included within the CAR encoding polynucleotide.

In one aspect, the engineered cell of this disclosure comprises, or alternatively consists essentially of, or yet further consists of a chimeric antigen receptor (CAR), wherein the anti-CD19 binding domain of the CAR comprises, or consists essentially of, or yet further consists of a single-chain variable fragment (scFv) that specifically recognizes a humanized anti-CD19 binding domain. In yet a further aspect, the engineered cell of this disclosure comprises, or alternatively consists essentially of, or yet further consists of a chimeric antigen receptor (CAR), wherein the anti-CD19 binding domain scFv of the CAR comprises, or alternatively consists essentially of, or yet further consists of a heavy chain variable region and a light chain variable region. Non-limiting examples of CAR extracellular domains capable of binding to antigens are the anti-CD19 binding domain sequences that specifically bind CD19 antigen as disclosed in the US20140271635 application.

In a particular embodiment, the engineered cell of this disclosure comprises, or alternatively consists essentially of, or yet further consists of a chimeric antigen receptor (CAR), wherein the anti-CD19 binding domain scFv of the CAR comprises, or alternatively consists essentially of, or yet further consists of a linker polypeptide located between the anti-CD19 binding domain scFv heavy chain variable region and the anti-CD19 binding domain scFv light chain variable region. In a further aspect, the engineered T cell of this disclosure comprises, or alternatively consists essentially of, or yet further consists of a chimeric antigen receptor (CAR), wherein the CAR linker polypeptide comprises, or alternatively consists essentially of, or yet further consists of the sequence (GGGGS)n wherein n is an integer from 1 to 6. Alternatively, a “linker sequence” relates to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In one aspect, the linker sequence is a (Glycine4Serine)3 flexible polypeptide linker comprising three copies of gly-gly-gly-gly-ser—represented in single letter sequence notation as GGGGS.

In a particular embodiment, the engineered cell of this disclosure comprises, or alternatively consists essentially of, or yet further consists of a chimeric antigen receptor (CAR), wherein the CAR further comprises, or alternatively consists essentially of, or yet further consists of a detectable marker or purification marker attached to or expressed by the CAR.

Non-limiting examples of detectable markers include enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

Non-limiting examples of purification markers include His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

In one aspect, the engineered cell of this disclosure comprises, or alternatively consists essentially of, or yet further consists of a polynucleotide encoding the CAR, and optionally, wherein the polynucleotide further encodes an anti-CD19 binding domain.

The CAR cells of this disclosure can be generated by inserting into the engineered immune cell a polynucleotide encoding the CAR and then expressing the CAR in the cell, Thus, in one aspect, the engineered T cell of this disclosure comprises, or alternatively consists essentially of, or yet further consists of a polynucleotide encoding the CAR, wherein the polynucleotide further comprises, or alternatively consists essentially of, or yet further consists of a promoter operatively linked to the polynucleotide to express the polynucleotide in the cell. Non-limiting examples of promoters include constitutive, inducible, repressible, or tissue-specific. The promoter is “operatively linked” in a manner to transcribe the linked polynucleotide.

In one aspect, the polynucleotide further comprises, or alternatively consists essentially of, or yet further consists of a sequence encoding a 2A self-cleaving peptide (T2A) that is optionally located upstream of a polynucleotide encoding an antigen binding domain, e.g., the anti-CD19 binding domain. “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P, wherein X refers to any amino acid generally thought to be self-cleaving.

In some embodiments, the engineered cell of this disclosure comprises, or alternatively consists essentially of, or yet further consists of a polynucleotide encoding the CAR, wherein the polynucleotide further comprises, or alternatively consists essentially of, or yet further consists of a signal peptide located upstream of a polynucleotide encoding the anti-CD19 binding domain. In a particular embodiment, the engineered cell of this disclosure comprises, or alternatively consists essentially of, or yet further consists of a polynucleotide encoding the CAR, wherein the polynucleotide further comprises, or alternatively consists essentially of, or yet further consists of a mouse Thy1.1 reporter signal polypeptide.

In some embodiments, the engineered cell of this disclosure comprises, or alternatively consists essentially of, or yet further consists of a polynucleotide encoding the CAR, wherein the polynucleotide further comprises, or alternatively consists essentially of, or yet further consists of the sequence, SEQ ID NO:1.

In some embodiments, the engineered T cell of this disclosure comprises, or alternatively consists essentially of, or yet further consists of a polynucleotide encoding the CAR, wherein the polynucleotide encodes the amino acid sequence of SEQ ID NO:2.

The polynucleotide encoding the CAR can be contained within a vector, e.g., a plasmid. In a separate aspect, the vector is a viral vector selected from the group of a retroviral vector, a lentiviral vector, an adenoviral vector, and an adeno-associated viral vector.

In some embodiments, the cell of this disclosure has been isolated from a subject. In a particular embodiment, the cell of this disclosure has been isolated from a subject, wherein the subject has cancer.

In a particular embodiment, the cell of this disclosure has been isolated from a subject, wherein the subject has cancer and the tumor antigen is expressed by a cell associated with the cancer.

### Producing the Cells

Also provided herein is a method of producing an engineered immune cell, the method comprising, or alternatively consisting essentially of, or yet further consisting of reducing or eliminating expression and/or function of an NR4A transcription factor in the cell. In one aspect, the method of producing an engineered immune cell further comprises, or alternatively consists essentially of, or yet further consists of isolating a cell from a subject, reducing or eliminating expression and/or function of an NR4A transcription factor in the cell and culturing the cell under conditions that favor expansion and proliferation of the cell.

Further provided herein is a method of producing an engineered immune cell, the method comprising reducing or eliminating expression and/or function of a TOX transcription factor in the cell. In one aspect, the method of producing an engineered immune cell further comprises, or alternatively consists essentially of, or yet further consists of isolating an immune cell from a subject, reducing or eliminating expression and/or function of a TOX transcription factor in said cell and culturing the immune cell under conditions that favor expansion and proliferation of the cell.

Immune cells include but are not limited to NK cells and T cells. As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

The term “reduce or eliminate expression and/or function of” intends reducing or eliminating the transcription of said polynucleotides into mRNA, or alternatively reducing or eliminating the translation of said mRNA into peptides, polypeptides, or proteins, or reducing or eliminating the functioning of said peptides, polypeptides, or proteins. In a non-limiting example, the transcription of polynucleotides into mRNA is reduced to at least half of its normal level found in wild type cells.

In one aspect, this disclosure provides a method of producing an engineered immune cell, the method comprising, or alternatively consisting essentially of, or yet further consisting of reducing or eliminating expression and/or function of an NR4A and a TOX transcription factor in the cell. In one aspect, the method of producing an engineered immune cell further comprises, or alternatively consists essentially of, or yet further consists of isolating an immune cell from a subject, reducing or eliminating expression and/or function of an NR4A transcription factor in said cell and culturing the cell under conditions that favor expansion and proliferation of the cell. In another aspect, the method of producing an engineered immune cell further comprises, or alternatively consists essentially of, or yet further consists of isolating an immune cell from a subject, reducing or eliminating expression and/or function of a TOX transcription factor in the cell and culturing the cell under conditions that favor expansion and proliferation of the cell.

The transduced cells can be cultured under conditions to grow and expand the cells.

For the purposes of the methods, the term “NR4A transcription factor” refers to the members of the NR4A subfamily of nuclear hormone receptors that bind to DNA and modulate gene expression. Non-limiting examples of members of NR4A transcription factor family are human NR4A1 (Nur77), NR4A2 (Nurr1) and NR4A3 (NOR1) encoded by the sequences provided in SEQ ID NO:3, SEQ ID NO:4 and SEQ ID NO:5, respectively. Also for the purposes of this disclosure, the term “TOX transcription factor” refers to the members of the TOX subfamily of nuclear hormone receptors that bind to DNA and modulate gene expression. Non-limiting examples of members of TOX transcription factor family are human TOX1, TOX2, TOX3 and TOX4 encoded by the sequences provided in SEQ ID NO:6, SEQ ID NO:7, SEQ ID NO:8 and SEQ ID NO:9, respectively. Further for the purposes of this disclosure, the term “IL-21” (interleukin 21) refers to the members of the common-gamma chain family of cytokines with immunoregulatory activity. A non-limiting example is human IL-21 encoded by the sequence provided in SEQ ID NO:10.

Immune cells include but are not limited to NK cells and T cells. As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

In another aspect, this disclosure provides a method of producing an engineered immune cell, the method comprising, or alternatively consisting essentially of, or yet further consisting of reducing or eliminating expression and/or function of an NR4A and a TOX transcription factor, and increasing the expression of IL-21 in the cell. In one aspect, the method of producing an engineered cell further comprises, or alternatively consists essentially of, or yet further consists of isolating an immune cell from a subject, reducing or eliminating expression and/or function of an NR4A transcription factor in said cell and culturing the cell under conditions that favor expansion and proliferation of the cell. In another aspect, the method of producing an engineered immune cell further comprises, or alternatively consists essentially of, or yet further consists of isolating an immune cell from a subject, reducing or eliminating expression and/or function of a TOX transcription factor in the cell and culturing the cell under conditions that favor expansion and proliferation of the cell. In a yet further aspect, the method of producing an engineered immune cell further comprises, or alternatively consists essentially of, or yet further consists of isolating an immune cell from a subject, increasing expression and/or function of IL-21 in the cell and culturing the cell under conditions that favor expansion and proliferation of the cell.

One of skill in the art can monitor expression of the transcription factors using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

One of skill in the art can use methods such as RNA interference (RNAi), CRISPR, TALEN, ZFN or other methods that target specific sequences to reduce or eliminate expression and/or function of NR4A or TOX transcription factors. CRISPR, TALEN, ZFN or other genome editing tools can also be used to increase expression and/or function of IL-21.

In a further aspect, a method of producing an engineered immune cell, the method comprising, or alternatively consisting essentially of, or yet further consisting of inhibiting expression and/or function of NFAT/AP-1 pathway in the cell. In one aspect, the method of producing an engineered immune cell further comprises, or alternatively consists essentially of, or yet further consists of isolating an immune cell from a subject, inhibiting expression and/or function of NFAT/AP-1 pathway in the cell and culturing the cell under conditions that favor expansion and proliferation of the cell.

As used herein, the term “inhibit expression and/or function of NFAT/AP-1 pathway” refers to reducing or eliminating the transcription of genes in the pathway, or alternatively reducing or eliminating the translation of said mRNA into pathway peptides, polypeptides, or proteins, or reducing or eliminating the functioning of said pathway peptides, polypeptides, or proteins. Non-limiting examples of inhibiting expression and/or function of NFAT/AP-1 pathway include inhibiting and/or function of NR4A transcription factor or TOX transcription factor, or alternatively increasing expression of IL-21.

The term “reduce or eliminate expression and/or function of” intends reducing or eliminating the transcription of said polynucleotides into mRNA, or alternatively reducing or eliminating the translation of said mRNA into peptides, polypeptides, or proteins, or reducing or eliminating the functioning of said peptides, polypeptides, or proteins. In a non-limiting example, the transcription of polynucleotides into mRNA is reduced to at least half of its normal level found in wild type cells.

Immune cells include but are not limited to NK cells and T cells. As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Culturesfy (https://www.dsmz.de/).

In a further aspect, the method of producing an engineered immune cell as described above further comprises, or alternatively consists essentially of, or yet further consists of isolating an immune cell from a subject, wherein the cell isolated from the subject binds a target antigen. In one aspect, the target antigen is a tumor antigen or antigens expressed by pathogens. In another aspect, the tumor antigen comprises, or alternatively consists essentially of, or yet further consists of mesothelin, ROR1, EGFRvIII, ephrin type-A receptor 2 (EphA2), interleukin (IL)-13r alpha 2, an EGFR VIII, a PSMA, an EpCAM, a GD3, a fucosyl GM1, a PSCA, a PLAC1, a sarcoma breakpoint, a Wilms Tumor 1, a hematologic differentiation antigen, a surface glycoprotein, a gangliosides (GM2), a growth factor receptor, a stromal antigen, a vascular antigen, or a combination thereof.

The term “receptor” or “T-cell receptor” or “TCR” refers to a cell surface molecule found on T-cells that functions to recognize and bind antigens presented by antigen presenting molecules. Generally, a TCR is a heterodimer of an alpha chain (TRA) and a beta chain (TRB). Some TCRs are comprised of alternative gamma (TRG) and delta (TRD) chains. T-cells expressing this version of a TCR are known as γδ T-cells. TCRs are part of the immunoglobulin superfamily. Accordingly, like an antibody, the TCR comprises three hypervariable CDR regions per chain. There is also an additional area of hypervariability on the beta-chain (HV4). The TCR heterodimer is generally present in an octomeric complex that further comprises three dimeric signaling modules CD3γ/ε, CD3δ/ε, and CD247 ζ/ζ or ζ/η. Non-limiting exemplary amino acid sequence of the human TCR-alpha chain: METLLGVSLVILWLQLARVNSQQGEEDPQALSIQEGENATMNCS YKTSINNLQWYRQNSGRGLVHLILIRSNEREKHSGRLRVTLDTSKKSSSLLITASRAA DTASYFCAPVLSGGGADGLTFGKGTHLIIQPYIQNPDPAVYQLRDSKSSDKSVCLFTD FDSQTNVSQSKDSDVYITDKTVLDMRSMDFKSNSAVAWSNKSDFACANAFNNSIIPE D TFFPSPESSCDVKLVEKSFETDTNLNFQNLSVIGFRILLLKVAGFNLLMTLRLWSS. Non-limiting exemplary amino acid sequence of the human TCR-beta chain: DSAVYLCASSLLRVYEQYFGPGTRLTVTEDLKNVFPPEVAVFEP PEAEISHTQKATLVCLATGFYPDHVELSWWVNGKEVHSGVSTDPQPLKEQP.

In one aspect the method of producing an engineered immune cell of this disclosure further comprises, or alternatively consists essentially of, or yet further consists of introducing into the cell a polynucleotide encoding a chimeric antigen receptor (polynucleotide CAR). In one embodiment, the polynucleotide CAR comprises, or alternatively consists essentially of, or yet further consists of a polynucleotide encoding: (a) an antigen binding domain; (b) a hinge domain; (c) a transmembrane domain; (d) and an intracellular domain.

In one aspect the method of producing an engineered immune cell of this disclosure further comprises, or alternatively consists essentially of, or yet further consists of introducing a polynucleotide CAR, wherein the polynucleotide CAR further comprises, or alternatively consists essentially of, or yet further consists of: (a) an anti-CD19 binding domain; (b) a hinge domain; (c) a CD28 or a CD8 α transmembrane domain; (d) one or more costimulatory regions selected from a CD28 costimulatory signaling region, a 4-1BB costimulatory signaling region, an ICOS costimulatory signaling region, and an OX40 costimulatory region; and (e) a CD3 zeta signaling domain.

A chimeric antigen receptor may optionally comprise a “hinge domain” which serves as a linker between the extracellular and transmembrane domains. Non-limiting exemplary polynucleotide sequences that encode for components are described herein.

Non-limiting examples of CAR extracellular domains capable of binding to antigens are the anti-CD19 binding domain sequences that specifically bind CD19 antigen as disclosed in the US20140271635 application.

In one particular aspect, the method of producing an engineered immune cell of this disclosure further comprises, or alternatively consists essentially of, or yet further consists of introducing a polynucleotide CAR, wherein the anti-CD19 binding domain of the polynucleotide CAR is a single-chain variable fragment (scFv) that specifically recognizes a humanized anti-CD19 binding domain. In yet a further aspect, the anti-CD19 binding domain scFv of the polynucleotide CAR comprises, or alternatively consists essentially of, or yet further consists of a heavy chain variable region and a light chain variable region.

A chimeric antigen receptor may optionally comprise a “hinge domain” which serves as a linker between the extracellular and transmembrane domains.

Non-limiting examples of CAR extracellular domains capable of binding to antigens are the anti-CD19 binding domain sequences that specifically bind CD19 antigen as disclosed in the US20140271635 application.

In one particular aspect, the method of producing an engineered T cell of this disclosure further comprises, or alternatively consists essentially of, or yet further consists of introducing a polynucleotide CAR, wherein the anti-CD19 binding domain scFv of the polynucleotide CAR comprises, or alternatively consists essentially of, or yet further consists of a linker polypeptide located between the anti-CD19 binding domain scFv heavy chain variable region and the anti-CD19 binding domain scFv light chain variable region. In a further aspect, the method of producing an engineered T cell of this disclosure further comprises, or alternatively consists essentially of, or yet further consists of introducing a polynucleotide CAR with a linker, wherein the polynucleotide CAR linker polypeptide comprises, or alternatively consists essentially of, or yet further consists of the sequence (GGGGS)n wherein n is an integer from 1 to 6.

In one aspect, the method of producing an engineered T cell of this disclosure further comprises, or alternatively consists essentially of, or yet further consists of introducing a polynucleotide CAR, wherein the polynucleotide further comprises a detectable marker and/or a purification marker.

In another aspect, the method of producing an engineered immune cell of this disclosure further comprises, or alternatively consists essentially of, or yet further consists of introducing a polynucleotide CAR, wherein the polynucleotide further comprises a promoter operatively linked to the polynucleotide to express the polynucleotide in said immune cell.

In a further aspect, the method of producing an engineered immune cell of this disclosure further comprises, or alternatively consists essentially of, or yet further consists of introducing a polynucleotide CAR, wherein the polynucleotide further comprises, or alternatively consists essentially of, or yet further consists of a 2A self-cleaving peptide (T2A) encoding polynucleotide sequence optionally located upstream of the polynucleotide encoding the anti-CD19 binding domain.

In some embodiments, the method of producing an engineered immune cell of this disclosure comprises, or alternatively consists essentially of, or yet further consists of introducing a polynucleotide CAR, wherein the polynucleotide sequence comprises, or alternatively consists essentially of, or yet further consists of SEQ ID NO:1.

In one embodiment, the method of producing an engineered immune cell of this disclosure comprises, or alternatively consists essentially of, or yet further consists of introducing a polynucleotide CAR, wherein the polynucleotide encodes the amino acid sequence of SEQ ID NO:2.

In another embodiment, the method of producing an engineered immune cell of this disclosure comprises, or alternatively consists essentially of, or yet further consists of introducing a polynucleotide CAR, wherein the polynucleotide further comprises, or alternatively consists essentially of, or yet further consists of a vector. In one aspect, the vector further comprises, or alternatively consists essentially of, or yet further consists of the isolated nucleic acid sequence comprising SEQ ID NO:1. In another aspect, the vector is a plasmid. In a separate aspect, the vector is a viral vector selected from the group of a retroviral vector, a lentiviral vector, an adenoviral vector, and an adeno-associated viral vector.

Also provided herein is an immune cell prepared by any of the methods of producing an engineered immune cell disclosed above.

Further provided herein is a substantially homogenous population of cells of any of the engineered immune cells of this disclosure.

Also provided herein is a heterogeneous population of cells of any of the engineered immune cells of this disclosure.

### Compositions

In one aspect, provided herein is a composition comprising, or alternatively consisting essentially of, or yet further consisting of a carrier and one or more of any of the engineered immune cells of this disclosure, or the population of cells of any of the engineered immune cells of this disclosure. In a further aspect, the carrier is a pharmaceutically acceptable carrier.

A “composition” typically intends a combination of the active agent, e.g., an engineered T-cell receptor, a modified T-cell receptor, a chimeric antigen receptor, a cell comprising an engineered T-cell receptor, a CAR T cell or a CAR NK cell, an antibody, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

In another aspect, provided herein is a composition comprising, or alternatively consisting essentially of, or yet further consisting of a carrier and one or more of any of the engineered immune cells of this disclosure, or the population of cells of any of the engineered immune cells of this disclosure, wherein the composition further comprises, or alternatively consists essentially of, or yet further consists of a cryoprotectant.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

“Cryoprotectants” are known in the art and include without limitation, e.g., sucrose, trehalose, and glycerol. A cryoprotectant exhibiting low toxicity in biological systems is generally used.

Provided herein is an immune cell bound to a target cell, wherein the immune cell is any of the engineered immune cells of this disclosure.

### Kits

Further provided is a kit comprising, or alternatively consisting essentially of, or yet further consisting of vectors and instructions for the manufacture of any of the engineered immune cells of this disclosure, and optionally, instructions for their use diagnostically or therapeutically.

### Methods of Use

Further provided herein is a method for stimulating a cell-mediated immune response to a target cell population, the method comprising, or alternatively consisting essentially of, or yet further consisting of contacting the target cell population with an engineered cell of this disclosure, or the population of cells of this disclosure. In a further aspect, a method for stimulating a cell-mediated immune response to a target cell population is provided, the method comprising, or alternatively consisting essentially of, or yet further consisting of contacting the target cell population with a cell of this disclosure, or the population of cells of this disclosure, wherein the contacting is in vitro or in vivo. The contacting can be direct or indirect binding or interaction between two or more entities (e.g., between target cell population and a T cell engineered to reduce or eliminate expression and/or function of a NR4A or TOX transcription factor in said cell). A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration. The target cell can be a pathogen infected cell or a cancer or tumor cell. In another aspect, the cancer is characterized as being hyporesponsive. In one aspect, the cell is selected for specific binding to the target cell. The cells can be from any species, e.g., a mammalian or a human cell. They can be isolated from a subject (e.g., from a biopsy) or a cultured cell.

In a further aspect, a method for stimulating a cell-mediated immune response to a target cell population is provided, the method comprising, or alternatively consisting essentially of, or yet further consisting of contacting the target cell population with a cell of this disclosure, or the population of cells of this disclosure, wherein the contacting is in vivo and the target cell population is a population of cancer cells in a subject. In another aspect, the cancer is characterized as being hyporesponsive.

Cancer cells targeted by this method include blood cancers such as acute myeloid leukemia or acute lymphoblastic leukemia, as well as solid tumors, e.g., a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma.

The methods are useful to treat humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). A mammal can be any age or at any stage of development (e.g., an adult, teen, child, infant, or a mammal in utero). A mammal can be male or female. In some embodiments, a human has or is suspected of having a cancer or neoplastic disorder. The method can be used as a first line, second line, third line, fourth line or fifth line therapy, and combined with other suitable therapies, e.g., surgical recession. In another aspect, the cancer is characterized as being hyporesponsive.

In yet another aspect, disclosed herein is a method for stimulating a cell-mediated immune response to a pathogen infected cell in a subject, the method comprising, or alternatively consisting essentially of, or yet further consisting of administering to the subject a cell of this disclosure or the population of cells of this disclosure in an amount effective to stimulate the cell-mediated immune response. In one aspect, the cell or population specifically binds the pathogen infected cell population. A pathogen infected cell population or pathogen infected cells treated by this method include, but are not limited to, infection by bacteria such as group A Streptococcus, Mycobacterium tuberculosis, Shigella flexneri, Salmonella enterica, Listeria monocytogenes, Francisella tularensis, and infection by viruses such as herpes simplex virus. The methods are useful to treat animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human.

In a further aspect, method for stimulating a cell-mediated immune response to a cancer target cell population, the method comprising, or alternatively consisting essentially of, or yet further consisting of administering to the subject a cells or population of cells of this disclosure in an amount effective to stimulate the cell-mediated immune response. In one aspect, the subject has, has had or is in need of treatment for cancer. In another aspect, the cancer is characterized as being hyporesponsive.

Also provided herein is a method of providing anti-tumor immunity in a subject, the method comprising, or alternatively consisting essentially of, or yet further consisting of administering to the subject a cell or population of cells of this disclosure, in an amount effective to provide the immunity to the subject. In one aspect, the subject is a mammal. In another aspect, the subject is a human. The cell or population are provided to preventing the symptoms or cancer from occurring in a subject that is predisposed or does not yet display symptoms of the cancer.

In one aspect disclosed herein is a method of treating a subject having a disease, disorder or condition associated with an elevated expression of a tumor antigen, the method comprising, or alternatively consisting essentially of, or yet further consisting of administering to the subject a cell or population of cells of this disclosure, in an amount effective to treat the subject. In another aspect, the tumor is characterized as being hyporesponsive.

Cancer cells targeted by these methods and tumor antigens associated with a cancer include antigens related to blood cancers such as acute myeloid leukemia or acute lymphoblastic leukemia, as well as solid tumors, e.g., a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. In one aspect, the cells or population specifically bind the cancer target cell population. In another aspect, the cancer is characterized as being hyporesponsive.

The methods are useful to treat humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). A mammal can be any age or at any stage of development (e.g., an adult, teen, child, infant, or a mammal in utero). A mammal can be male or female. In some embodiments, a human has or is suspected of having a cancer or neoplastic disorder. The method can be used as a first line, second line, third line, fourth line or fifth line therapy, and combined with other suitable therapies, e.g., surgical recession.

In certain embodiments a subject has or is suspected of having a neoplastic disorder, neoplasia, tumor, malignancy or cancer. In some embodiments a subject in need of a treatment, cell or composition described herein has or is suspected of having a neoplastic disorder, neoplasia, tumor, malignancy or cancer.

In one aspect, a method for stimulating a cell-mediated immune response to a pathogen-infected target cell population in a subject is provided, the method comprising, or alternatively consisting essentially of, or yet further consisting of administering to the subject a cell of this disclosure or the population of cells of this disclosure. In one aspect, the subject has, has had or is in need of treatment for a pathogen infection. A pathogen infected cell population or pathogen infected cells refer treated by this method include, but are not limited to, infection by bacteria such as group A Streptococcus, Mycobacterium tuberculosis, Shigella flexneri, Salmonella enterica, Listeria monocytogenes, Francisella tularensis, and infection by viruses such as herpes simplex virus. Subjects treated by this method includes animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. The method can be combined with other suitable therapies or treatments.

In some embodiments a subject is in need of a treatment, cell or composition described herein. In certain embodiments a subject has or is suspected of having a pathogen infection. In some embodiments a subject in need of a treatment, cell or composition described herein has or is suspected of having a pathogen infection.

For the above methods, an effective amount is administered, and administration of the cell or population serves to attenuate any symptom or prevent additional symptoms from arising. When administration is for the purposes of preventing or reducing the likelihood of cancer recurrence or metastasis or pathogen infection, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

The methods provide one or more of: (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies. In one aspect, treatment excludes prophylaxis.

Also provided herein is a method of providing immunity to the pathogen infection in a subject, the method comprising, or alternatively consisting essentially of, or yet further consisting of administering to the subject any of a cell or population of this disclosure, in an amount that provides immunity. In one aspect, the methods prevent the symptoms or pathogen infection from occurring in a subject that is predisposed or does not yet display symptoms of the pathogen infection. The methods are useful to treat animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. The methods can be combined with other suitable therapies and can be used for the treatment of virus, bacteria, fungi, and protozoa. Examples of pathogenic infections include, but are not limited to, infection by bacteria such as group A Streptococcus, Mycobacterium tuberculosis, Shigella flexneri, Salmonella enterica, Listeria monocytogenes, Francisella tularensis, and infection by viruses such as herpes simplex virus.

### DISCUSSION

Many existing methods for engineering adoptive T cell therapies are focused on forcing T cells to recognize the tumor antigens by expressing specific T cell receptors or chimeric antigen receptors. After infusion, the function of cells will be affected after entering the tumor environment. From in vitro models, the inventors have found that conventional T cells and CAR-T cells rapidly acquire a hyporesponsive, “exhausted” state and poorly clear implanted melanoma tumors. The present disclosure provides that CAR-T cells genetically engineered to lack Nr4a transcription factors retain function within tumors and promote tumor clearance.

This “exhausted” state is characterized by a unique gene expression and epigenetic profile, including increased activity and altered expression of the Nr4a family transcription factors (Nr4a1 aka Nur77, Nr4a2 aka Nurr1, Nr4a3 aka Nor1). Using an implantable melanoma mouse model, the inventors found a similar “exhaustion” profile among endogenous, polyclonal tumor infiltrating T cells, adoptively transferred monoclonal transgenic T cells, and chimeric antigen receptor expressing T (CAR-T) cells. CAR-T cells engineered to lack Nr4a1, Nr4a2 and Nr4a3 transcription factors cleared implanted melanoma tumors, while unmodified CAR-T cells did not limit tumor growth. The Nr4a-deleted CAR-T cells produced greater amounts of IFN-gamma and TNF upon restimulation than Nr4a-expressing CAR-T cells. Thus, deletion of Nr4a is a potential strategy to improve outcomes after adoptive cell therapy, particularly for CAR-T cells directed against solid tumors.

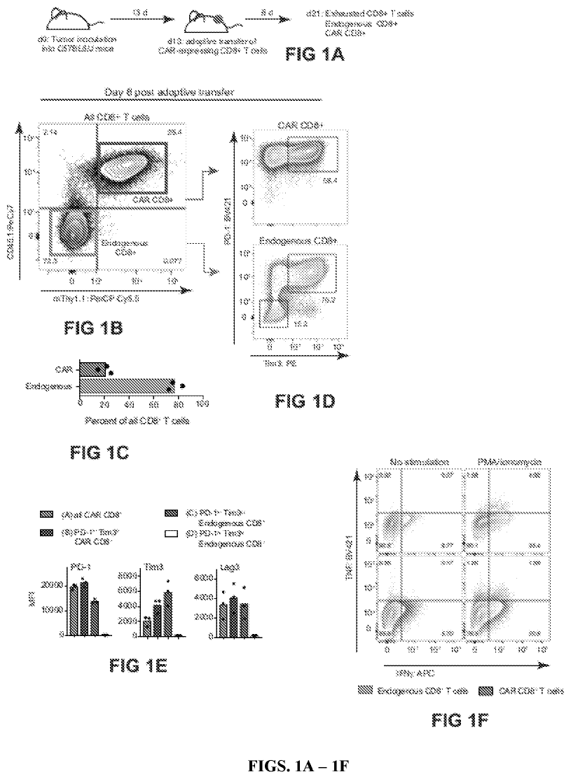

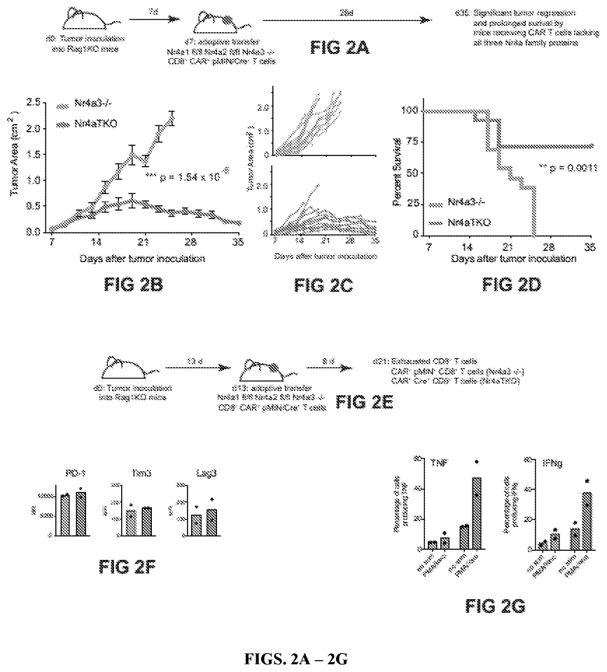

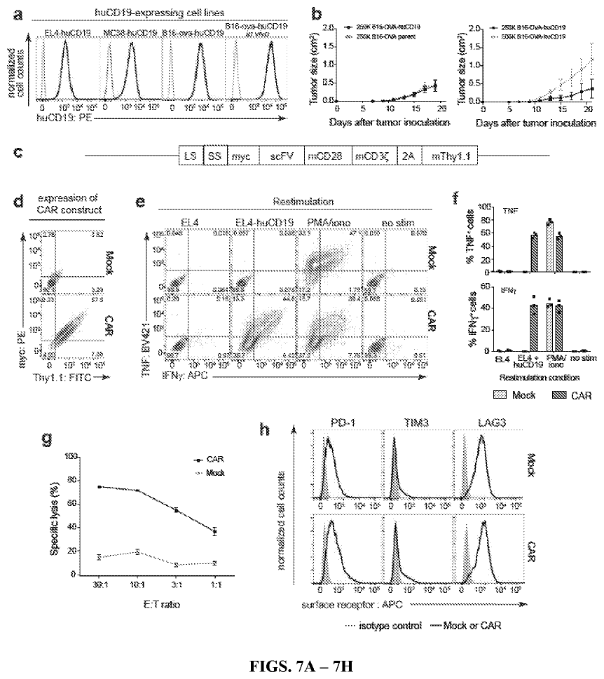

To identify changes in gene expression and associated regulatory elements in CAR T cells infiltrating solid tumors, and compared the properties of three distinct classes of CD8⁺ tumor-infiltrating lymphocytes (TILs): CAR TILs that recognize human CD19 (huCD19), OT-I T cell receptor (TCR)-transgenic TILs (OT-I TILs) that recognize the SIINFEKL peptide from chicken ovalbumin (OVA) presented by H-2K^{b}, and endogenous TILs from the recipient congenic mice (FIG. 7). As targets for the CART cells, the B16-OVA mouse melanoma cell line, the EL4 mouse thymoma cell line, and the MC38 colon adenocarcinoma cell line to express human CD19 (huCD19) were engineered (FIG. 1A, left panels); the resulting B16-OVA-huCD19 cells are recognized by OVA-reactive OT-I as well as by huCD19-reactive CAR-expressing CD8⁺ T cells. It was confirmed that the B16-OVA-huCD19 cell line stably maintained huCD19 expression after subcutaneous growth in syngeneic C57BL/6J mice for 18 days and subsequent culture for 7 days ex vivo (FIG. 7A, right panel). The Bf16-OVA and B16-OVA-huCD19 cells grew at the same rate in vivo, indicating that addition of the huCD19 antigen did not cause rejection of the tumor cells (FIG. 1B, left). Based on the growth rate of the tumors in mice, mice were inoculated with 500,000 B16-OVA-huCD19 tumor cells (FIG. 7B, right).

The CAR T cells express a second-generation CAR in which a myc epitope-tagged single-chain variable fragment specific for huCD19^{22,23} was fused to the transmembrane domain of murine CD28 and the intracellular signaling portions of mouse CD28 and CD3ζ¹, and was followed in the retroviral construct by a 2A self-cleaving peptide and a mouse Thy1.1 reporter (FIG. 7C). 95.5±4.0% transduction efficiency of the CAR retrovirus in mouse CD8⁺ T cells was achieved (FIG. 1D). The CAR T cells were functional, as they produced the cytokines TNF and IFNγ upon restimulation with EL4-huCD19 target cells in culture (FIG. 7E, 7F), and exhibited dose-dependent target cell lysis against the B16-OVA-huCD19 cells in vitro (FIG. 7G). The CART cells resembled mock-transduced cells in their surface expression of PD-1, TIM3, and LAG3 under resting conditions (FIG. 7H).

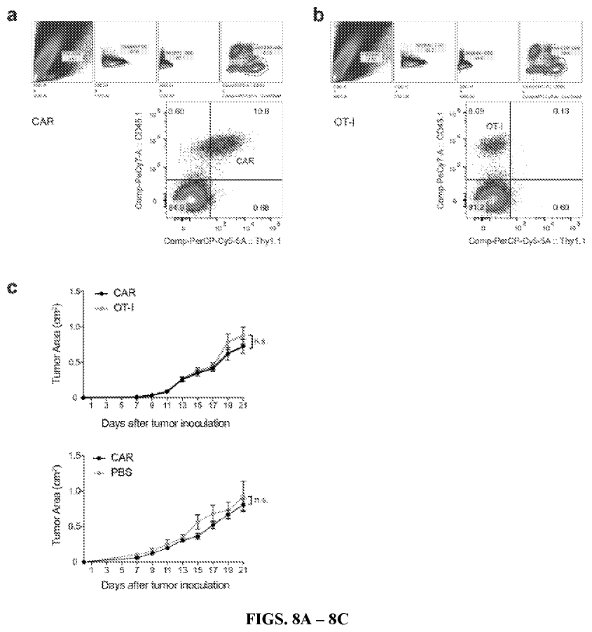

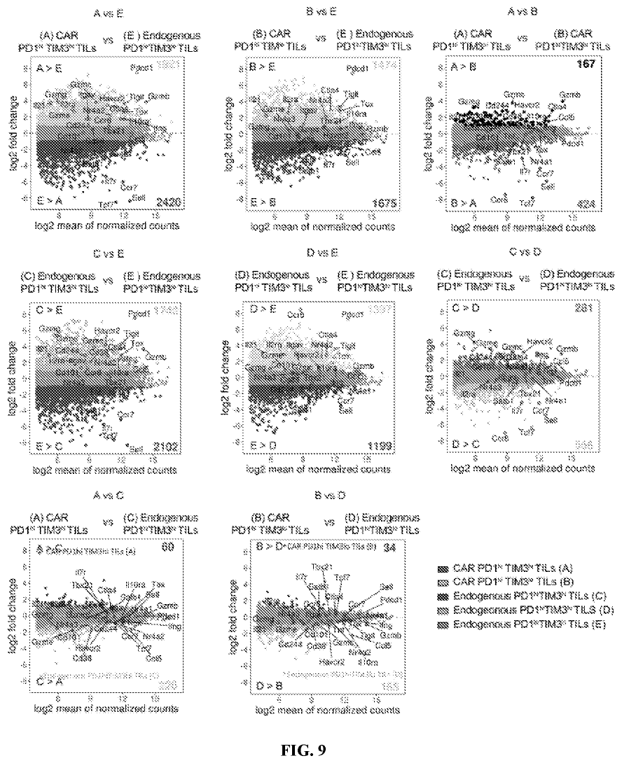

To assess CAR T cell function, CD45.1⁺ CD8⁺ T cells were transduced with the CAR retrovirus and adoptively transferred into C57BL/6J mice bearing B16-OVA-huCD19 tumors, 13 days after tumor inoculation (FIG. 7A). Eight days after adoptive transfer, CD8⁺ CD45.1⁺ Thy1.1⁺ CAR TILs as well as endogenous CD45.2⁺ host T cells were isolated (FIG. 7B, left). For comparison, a parallel analysis with adoptively transferred CD45.1⁺ CD8⁺ OT-I TCR-transgenic cells¹⁸ infiltrating the same B16-OVA-huCD19 tumor was performed (FIG. 1C; 1D; for gating scheme see FIG. 8A, 8B). Tumor growth rates were comparable in mice that received CAR or OT-I CD8⁺ T cells (FIG. 8C, top panel), allowing direct comparison of these two TIL populations; the number of transferred CAR T cells was kept low to minimize tumor rejection (FIG. 8C, bottom panel), allowing the effects of genetic manipulations to be more easily observed. On average, the CAR and OT-I T cells comprised ˜18% and ˜9% of CD8⁺ TILs in the tumor (FIG. 7E).

At 8 days after transfer, all three TIL populations produced low levels of the cytokines TNF and IFNγ upon restimulation (FIG. 7F, 7G), confirming their decreased function. All three TIL populations also contained PD-1^{high} TIM3^{high} cells that are thought to be highly exhausted (populations A, C and F in FIG. 7B, 7D, top right), as well as PD-1^{high} TIM3^{low} cells, thought to be antigen-specific memory precursors that proliferate after treatment with anti-PD-1/PD-L1 (populations B and D). The endogenous TILs also contained a population of PD-1^{low} TIM3^{low} T cells (FIG. 7B, 7D, lower right) that resembled naïve T cells (see ATAC-seq data below). Thus, all three TIL populations—CAR, OT-I and endogenous—developed similar phenotypes of decreased cytokine production and increased inhibitory receptor expression.

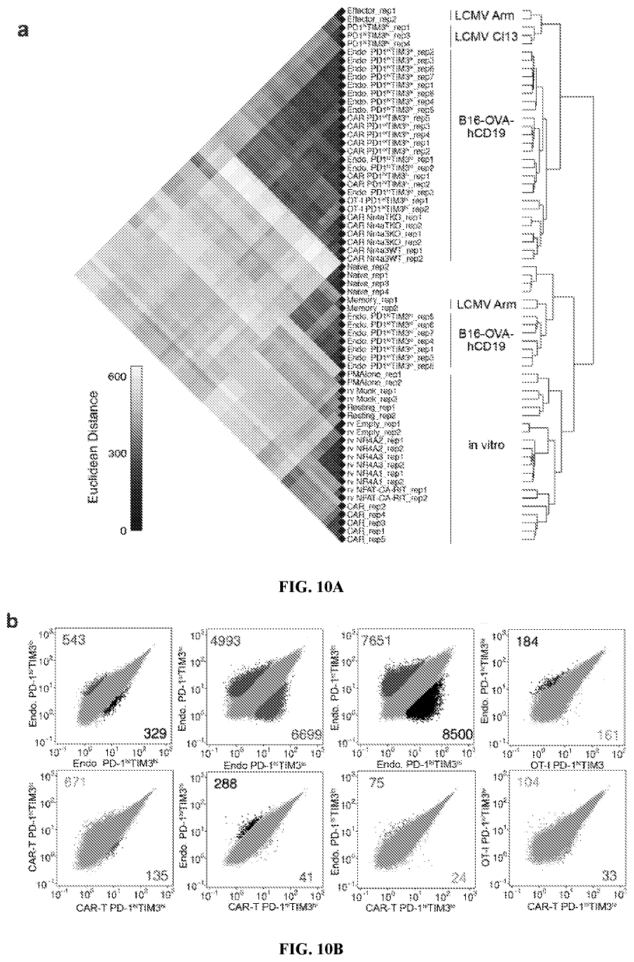

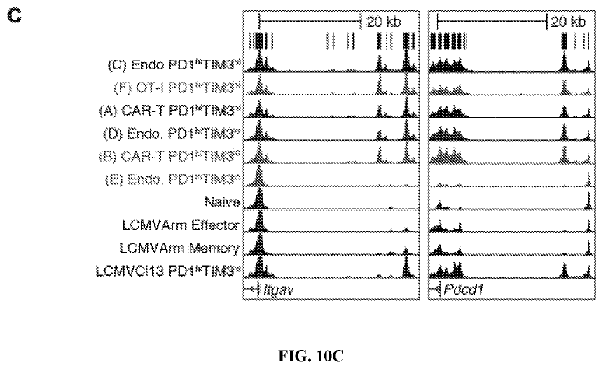

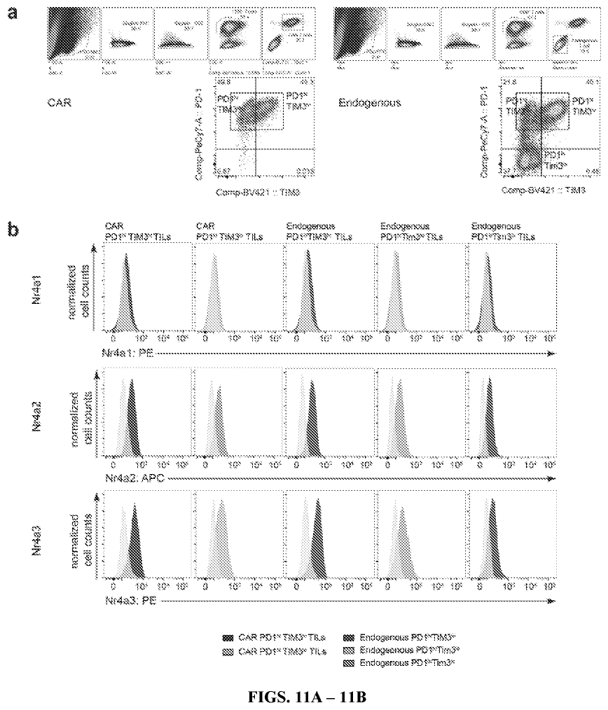

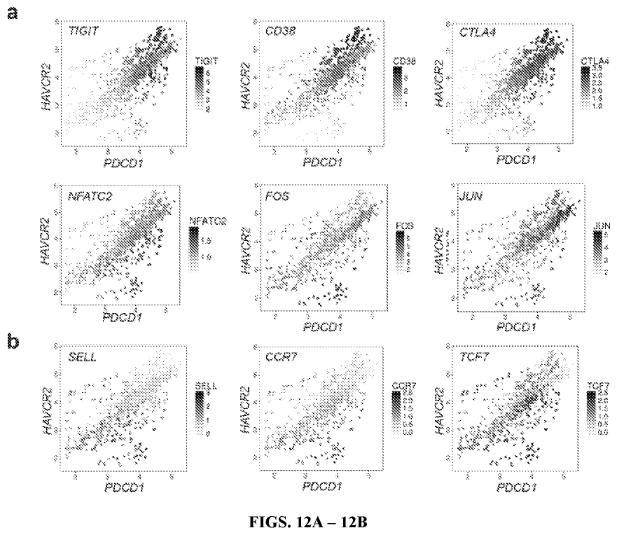

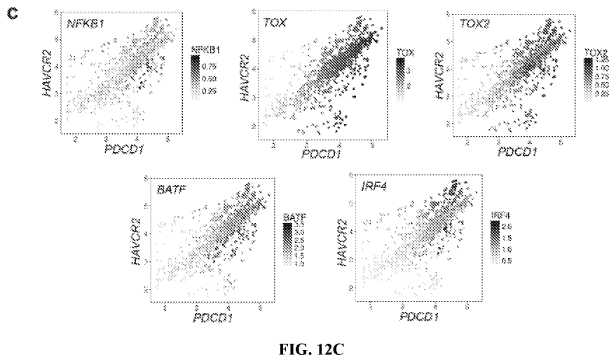

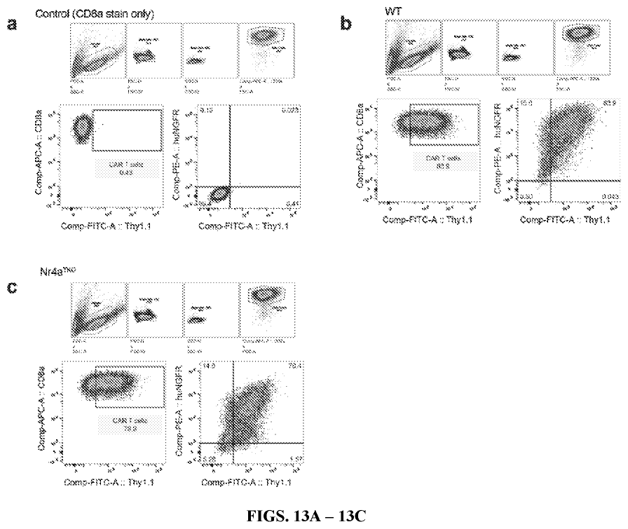

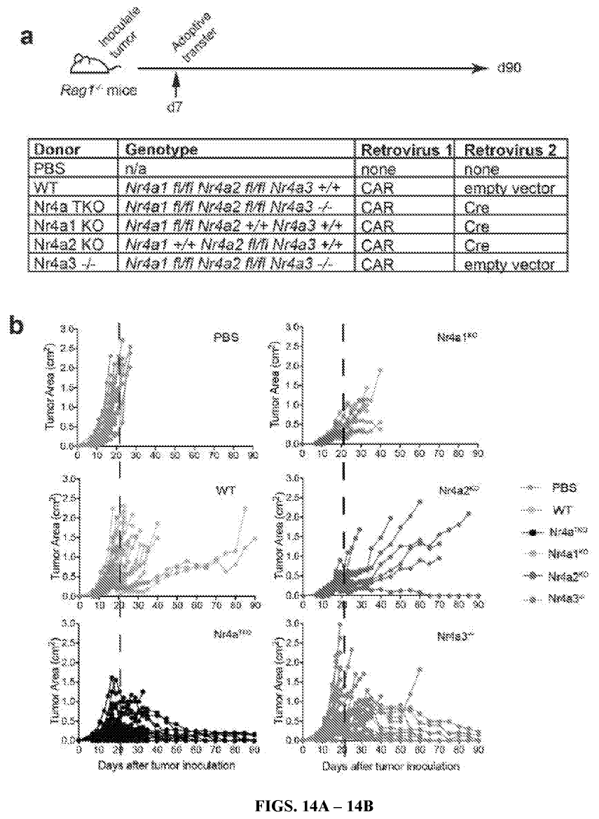

RNA-sequencing²⁴ (RNA-seq) and ATAC-seq²⁵ (Assay for transposase-accessible chromatin followed by sequencing) was used to compare the gene expression and chromatin accessibility profiles²⁶⁻²⁸ of the different populations (A-F) of CD8⁺ tumor-infiltrating lymphocytes in the CAR T cell system (FIG. 7B, 7D). Principal component analysis of the RNA-seq data showed that the transcriptional profiles of PD-1^{high} TIM3^{high} CAR TIL populations (A) were similar to those of endogenous PD-1^{high} TIM3^{high} TILs (C), but distinct from those of CAR and endogenous PD-1^{high} TIM3^{low} cells (B, D) and endogenous PD-1^{low} TIM3^{low} cells (E) (FIG. 8A, FIG. 9). Similarly, ATAC-seq analysis showed that the genome-wide chromatin accessibility profiles of the endogenous, OT-I and CAR PD-1^{high} TIM3^{high} and PD-1^{high} TIM3^{low} TIL subsets were similar to one another, but distinct from those of PD-1^{low} TIM3^{low} endogenous TILs (FIG. 8B, FIG. 10).

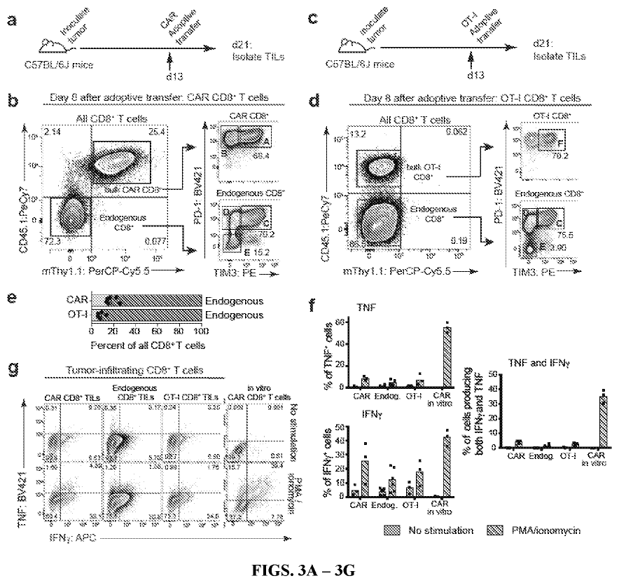

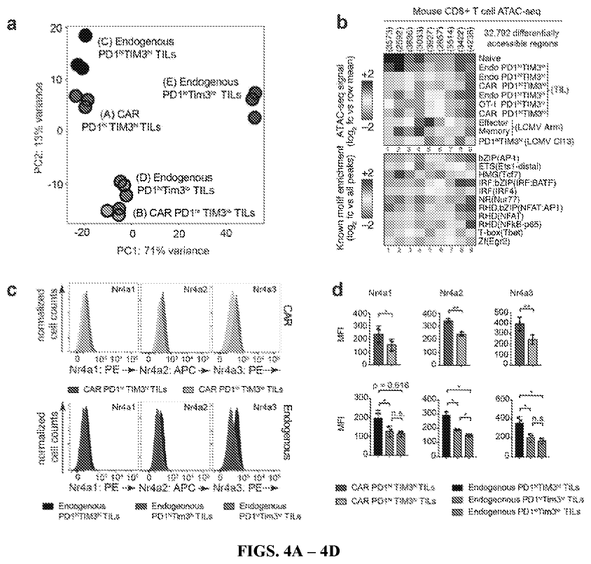

The B16-OVA melanoma model that tumor-reactive OT-I TILs (PD-1^{high} TIM3^{high}) showed increased expression of genes encoding the transcription factors Nr4a2 and Tox, various inhibitory surface receptors, and effector genes including granzymes and cytokines, compared to PD-1^{low} TIM3^{low} tumor-nonreactive P14 TILs¹⁸. Consistent with these findings, genes encoding transcription factors (Nr4a2, Tox, Tbx21), inhibitory receptors (Pdcd1, Ctla4, Havrc2, Tigit), several granzymes, cytokines (Il21, Ifng, Tnf), and cytokine receptors (Il2ra, Il10ra) were also upregulated in PD-1^{high} CAR TILs and endogenous TILs (populations A-D), compared to endogenous PD-1^{low} TIM3^{low} TILs (population E) (FIG. 9, panels comparing populations A vs E, B vs E, C vs E and D vs E). Exhausted PD-1^{high} TIM3^{high} CAR and endogenous TILs (populations A, C) did not show significant differences in Nr4a mRNA expression compared to PD-1^{high} TIM3^{low} (populations B, D) (FIG. 9, panels comparing populations A vs B, C vs D), but Nr4a protein expression was clearly increased in the former TIL populations (A, C) compared to the latter (B, D) (see below). Finally, consistent with the published literature^{9,17,18}, the PD-1^{high} TIM3^{low} antigen-specific memory precursors resembled naïve CD8⁺ T cells in expressing higher levels of Tcf7, Il7r, Ccr7 and Sell (encoding CD62L (L-selectin)) mRNAs compared to exhausted PD-1^{high} TIM3^{high} TILs (FIG. 3, panels comparing populations A vs E, B vs E, C vs E and D vs E).

Analysis of the ATAC-seq data showed that endogenous PD-1^{low} TIM3^{low} TILs (population E) resembled naïve CD8⁺ T cells in their pattern of chromatin accessibility and were distinct from PD-1^{high} TIM3^{high} and PD-1^{high} TIM3^{low} TILs (populations A-D) (FIG. 8B, top panel; FIG. 10). Moreover, regions selectively accessible in populations A-D (PD-1^{high} CAR and endogenous TILs) were enriched for consensus Nr4a binding motifs, as well as consensus binding motifs for NFAT, NFκB, bZIP and IRF-bZIP motifs (FIG. 8B, clusters 8 and 9; discussed below). Again consistent with the published literature^{9,17,18}, the chromatin accessibility profile of memory-precursor PD-1^{high} TIM3^{low} TILs (populations B, D) resembled that of naïve CD8⁺ T cells in showing substantial enrichment for Tcf7 binding motifs (FIG. 8B, cluster 6).

As mentioned above, PD-1^{high} TIM3^{high} and PD-1^{high} TIM3^{low} TIL populations displayed no significant difference in expression of Nr4a family members at the mRNA level (FIG. 9, panels comparing populations A vs B, C vs D). However, flow cytometric analysis showed clearly that at the protein level, expression of all three Nr4a transcription factors was higher in PD-1^{high} TIM3^{high} compared to PD-1^{high} TIM3^{low} TIL populations. Together, the increased expression of Nr4a family members in exhausted PD-1^{high} TIM3^{high} compared to PD-1^{high} TIM3^{low} TIL populations, as well as the enrichment for Nr4a binding motifs in the differentially accessible regions of these cells, pointed strongly to Nr4a family members as potential transcriptional effectors of the CD8⁺ T cell response to chronic antigen stimulation.

Data from human TILs and T cells from chronically infected HIV patients provided further justification for our focus on the Nr4a family. Single-cell RNA-seq data derived from CD8⁺ T cells infiltrating a human melanoma²⁰ revealed that NR4A1 and NR4A2 expression showed a strong positive correlation with PDCD1 (encoding PD-1) and HAVCR2 (encoding TIM3) expression, whereas NR4A3 showed a moderate positive correlation (FIG. 8E). Consistent with our mouse RNA-seq data, a positive correlation of PDCD1 and HAVCR2 mRNA expression with expression of mRNAs encoding the surface receptors CD38, TIGIT, and CTLA4 was observed (FIG. 12A); and a negative correlation with expression of mRNAs encoding the transcription factor TCF1, the cell surface receptor SELL (L-selectin, or CD62L), and the chemokine receptor CCR7 (FIG. 12B). The expression of mRNAs encoding other transcription factors of interest—TOX, TOX2 and IRF4—also correlated positively with the expression of PDCD1 and HAVCR2 mRNAs (FIG. 12C). Analysis of human ATAC-seq data^{19,21} showed enrichment for Nr4a nuclear receptors, NFAT, bZIP and IRF:bZIP motifs in regions uniquely accessible in CD8⁺ PD-1^{high} TILs from melanoma and non-small cell lung cancer, and HIV-antigen specific CD8⁺ T cells from infected humans. Taken together, FIG. 12 shows that Nr4a family members are upregulated, and Nr4a nuclear receptor binding motifs are enriched in regions accessible to PD-1^{hi} T cells^{17,18,21,29} in both human and mouse CD8⁺ T cells exposed to chronic antigen stimulation.

All three members of the Nr4a family are essential for the development of regulatory T cells³⁰, suggesting that they may function redundantly in other biological contexts. Considering this expected redundancy, Nr4a-sufficient (WT) CAR TILs was compared with CAR TILs triply deficient in all three Nr4a transcription factors (Nr4a TKO) (FIG. 9). Because the Nr4a gene-disrupted mice were originally derived from 129/SvJ ES cells³¹, and their genetic background might not have been fully compatible with that of inbred C57BL/6J mice despite stringent backcrossing, Rag1-deficient mice as recipients for tumor inoculation to avoid variable rejection of the CART cells. Naïve CD8+ T cells from Nr4a1 fl/fl Nr4a2 fl/fl Nr4a3−/− mice were simultaneously transduced with two retroviruses, the first encoding the CAR-2A-Thy1.1 (FIG. 7C) and the second encoding Cre followed by an IRES-NGFR cassette, to yield Nr4a triple knockout (Nr4a TKO) CAR T cells (FIG. 7A). As controls, naïve CD8⁺ T cells from Nr4a1 fl/fl Nr4a2 fl/fl Nr4a3+/+ mice were retrovirally transduced with the CAR-2A-Thy1.1 retrovirus and the empty retrovirus with IRES-NGFR alone, to yield Nr4a WT CAR T cells (WT) (FIG. 13A). The CAR T cells were adoptively transferred into mice that had been injected with B16-OVA-huCD19 melanoma cells 7 days previously, and tumor growth was monitored for an additional 83 days. Compared to control mice adoptively transferred with WT CD8⁺ CAR T cells, mice adoptively transferred with Nr4a TKO CD8⁺ CAR T cells lacking all three Nr4a transcription factors showed pronounced tumor regression and enhanced survival (FIG. 9B, 9C), with the tumor size difference between the three groups (PBS, WT and Nr4a TKO) apparent as early as day 21 after tumor inoculation (i.e. 14 days after adoptive transfer) (FIG. 9B, bottom panel). Thus, Nr4a transcription factors suppress tumor rejection in the CAR T cell model.

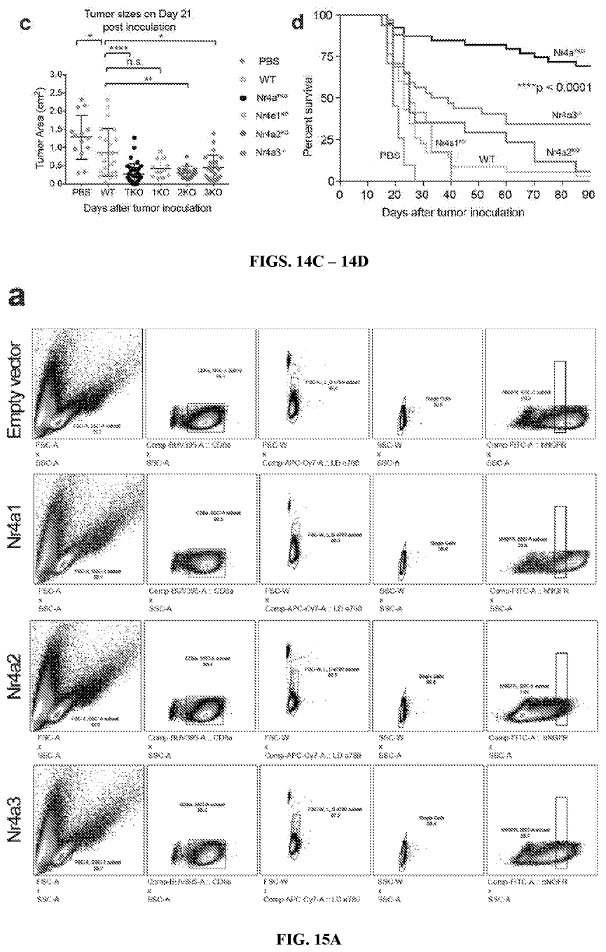

To explore the redundancy of the Nr4a family members in CD8⁺ T cell function in vivo, the anti-tumor effects of CD8⁺ CAR T cells lacking individual Nr4a proteins were compared to those of WT and Nr4a TKO CAR T cells (FIG. 14). The CAR T cells were adoptively transferred into Rag1-deficient mice that had been injected with B16-OVA-huCD19 melanoma cells 7 days previously, and tumor growth was monitored for an additional 83 days (FIG. 14A). Tumor growth curves and survival curves showed that Nr4a TKO CAR T cells exhibited superior anti-tumor activity compared to CAR T cells from mice lacking any single Nr4a protein (FIG. 14B, 14C). Again, tumor size differences between the WT and the various Nr4a knockouts were observed as early as day 21 after tumor inoculation.

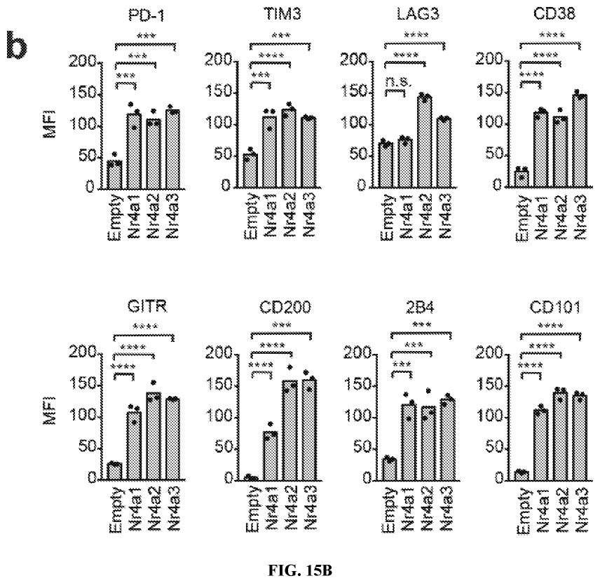

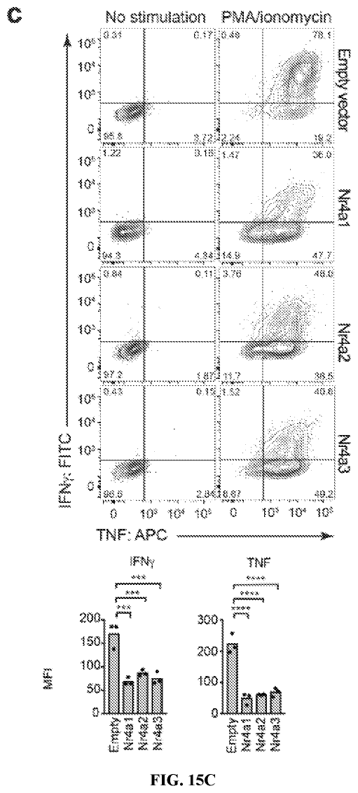

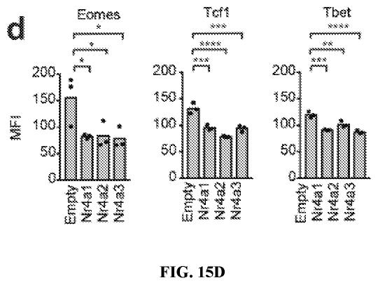

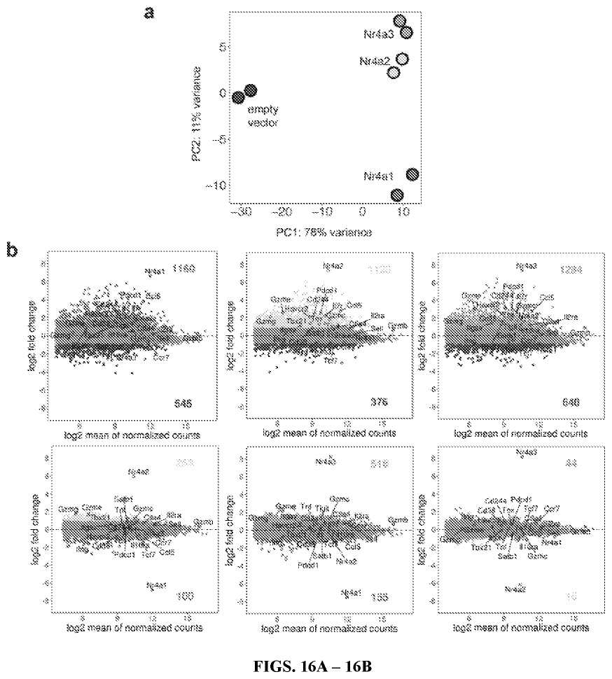

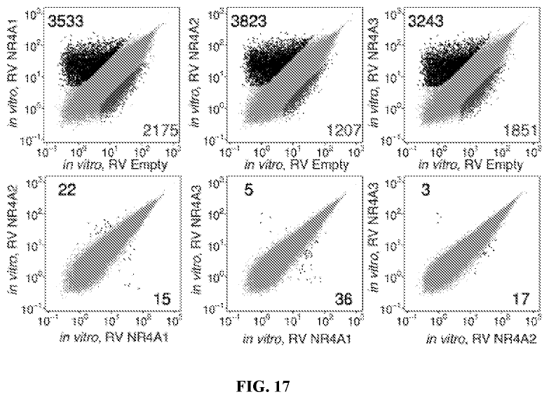

To further confirm the redundant functions of the three Nr4a family members in CD8⁺ T cell function, retroviruses to express Nr4a1, Nr4a2, and Nr4a3 in mouse CD8+ T cells in vitro were used (FIG. 15-17). Ectopic expression of any of the three Nr4a transcription factors resulted in increased expression of inhibitory surface receptors PD-1, TIM3, 2B4 and GITR and decreased production of the cytokines TNF and IFN_{γ} upon restimulation (FIG. 15). Principal component analysis of the RNA-seq data of cells expressing any given Nr4a transcription factor or the empty vector control indicated that the majority of the variance between these groups was at genes with a similar profile in cells expressing Nr4a family members compared to empty vector (FIG. 16A). In both RNA-seq and ATAC-seq, pairwise comparisons showed very few if any differences between Nr4a family members (FIG. 16B, 17A). Thus all three Nr4a proteins induce overlapping changes in gene expression and regulatory element accessibility profiles of CD8⁺ T cells in vitro as well as in vivo.

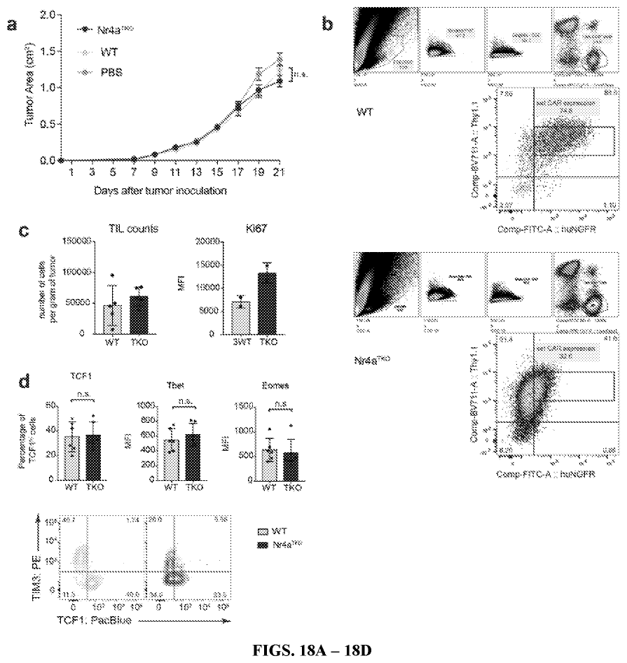

To assess the phenotypic and genome-wide changes associated with anti-tumor function in Nr4a TKO and WT, experimental conditions were modified to delay tumor regression (FIG. 9D) and ensure similar tumor sizes between the two groups (FIG. 18A), for gating scheme of TILs see FIG. 18B). The number of Nr4a TKO TILs recovered per gram of tumor was not significantly different from the number of WT TILs recovered (FIG. 18C). At day 21 following tumor inoculation (8 days after adoptive transfer), there was a mild but statistically significant decrease in PD-1 expression when comparing WT and Nr4a TKO TILs, as well as a striking skewing of the total PD-1^{high} population towards low TIM3 expression (FIG. 9E, top panels); in the Nr4a TKO TILs, there is a skewing of the TIM3^{low} populations towards low TCF1 expression (FIG. 18D, bottom). In tests of effector function, the percentage of cells expressing TNF and both IFN_{γ} and TNF was significantly higher in Nr4a TKO compared to WT TILs (FIG. 9F). On a bulk level, there was no significant difference in MFI of TCF1, Tbet, or Eomes (FIG. 18D, top).

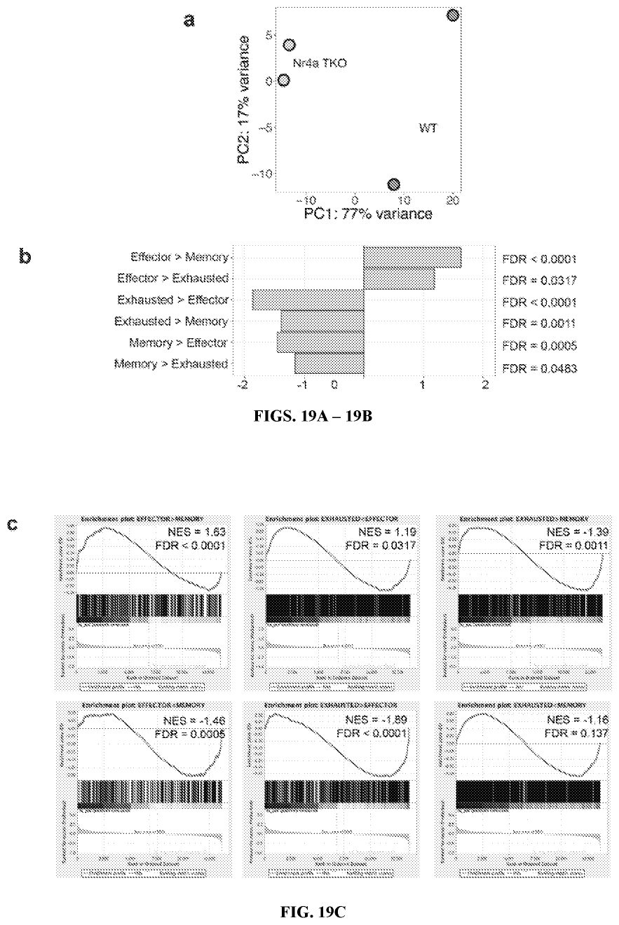

Genome-wide changes associated with effector function in the Nr4a TKO and WT TILs by RNA-seq and ATAC-seq. RNA-seq identified 1,076 differentially expressed genes, of which 536 genes were more highly expressed in the Nr4a TKO TILs and 540 were more highly expressed in the WT TILs (FIG. 10A, FIG. 19A). Gene set enrichment analysis³² (GSEA) using gene sets from effector, memory and exhausted (PD-1^{high} TIM3^{high}) populations isolated from LCMV-infected mice¹⁷ showed that broadly, Nr4a TKO TILs were enriched for genes related to effector function (FIG. 10A; FIG. 19B, 19C). Specifically, mRNAs encoding IL-2Rα, TNF, and granzymes were upregulated in Nr4a TKO TILs, consistent with the increased production of TNF observed upon restimulation (FIG. 9G). In contrast, genes that are typically upregulated in naïve/memory T cells compared to effector populations, such as Sell (encoding L-selectin/CD62L) and Ccr7 were downregulated in Nr4a TKO compared to WT TILs (FIG. 10A). Inhibitory surface receptors usually upregulated in hyporesponsive T cells, including Pdcd1, Havcr2, Cd244, Tigit, and Cd38, were also downregulated in Nr4a TKO compared to WT TILs (FIG. 10A).

To identify the transcriptional targets of individual Nr4a proteins, genes differentially expressed in Nr4aTKO TILs compared to WT TILs (FIG. 10A) were considered, and clustered by whether their expression changed when Nr4a was ectopically expressed (FIG. 10B). Clusters 1 and 2 contain genes that are downregulated in the absence of Nr4a, and upregulated in cells ectopically expressing Nr4a—these include Pdcd1, Havcr2, Cd244 in cluster 1, and Tox, Tigit and Cd38 in cluster 2. Cluster 4 contains genes, notably Tnf and Il21, that were upregulated in the absence of Nr4a, and downregulated in cells ectopically expressing Nr4a. Notably, Runx3 was not among the genes differentially expressed in Nr4a TKO compared to WT TILs, even though previous publications have identified it as a downstream target of Nr4a in the context of CD8⁺ T cell development³³, and as a gene whose overexpression contributes to tumor regression³⁴.

ATAC-seq revealed ˜2500 differentially accessible regions between WT and Nr4a TKO TILs (FIG. 10C). Among regions lost in Nr4a TKO TILs, a substantial fraction (˜36%) contained Nr4a binding motifs and a smaller subset contained NFAT binding sites (FIG. 10C). Of regions more accessible in Nr4a TKO compared to WT TILs, ˜71% were enriched for consensus bZIP family motifs and 25% for consensus Rel/NFκB binding motifs, confirming the established role of bZIP (Fos, Jun, ATF, CREB, etc) and Rel/NFκB family members in T cell activation and effector function. These data are also consistent with a previous publication suggesting a negative crosstalk between Nr4a and NFκB^{35,36}. Thus, Nr4a TKO TILs display potent effector function by several independent measures: altered profiles of gene expression that confer decreased expression of inhibitory receptors and increased cytokine production; and strong enrichment of binding motifs for transcription factors involved in effector function in regions of accessible chromatin.

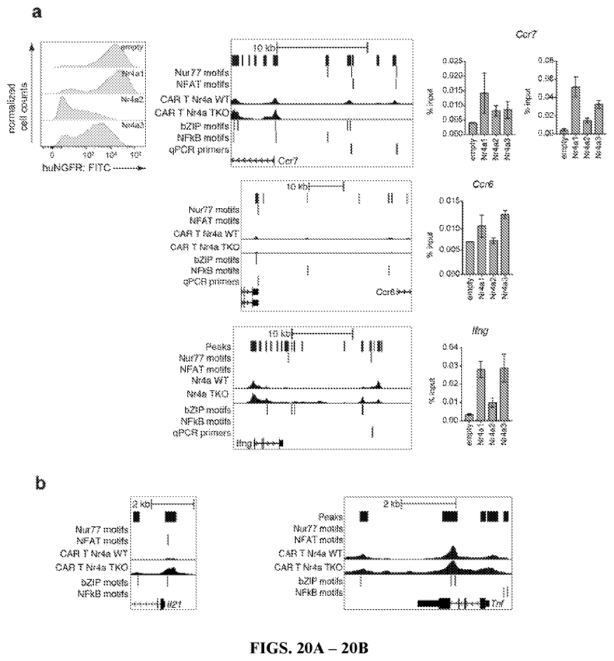

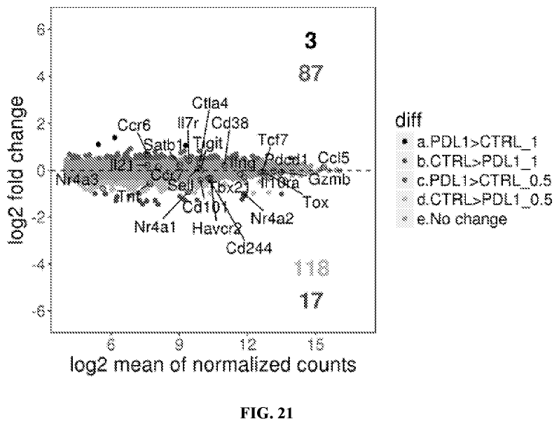

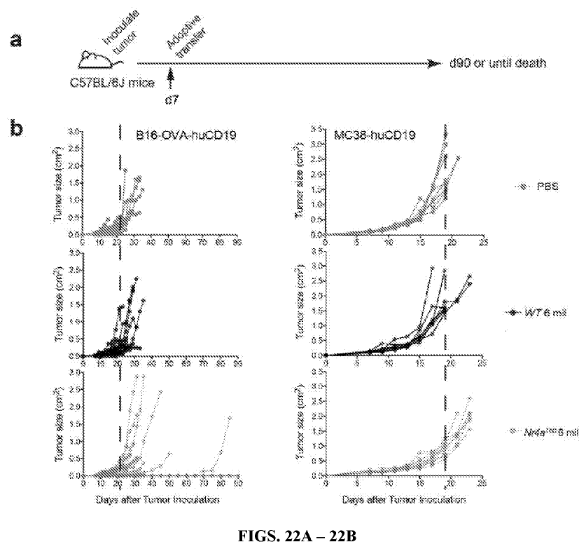

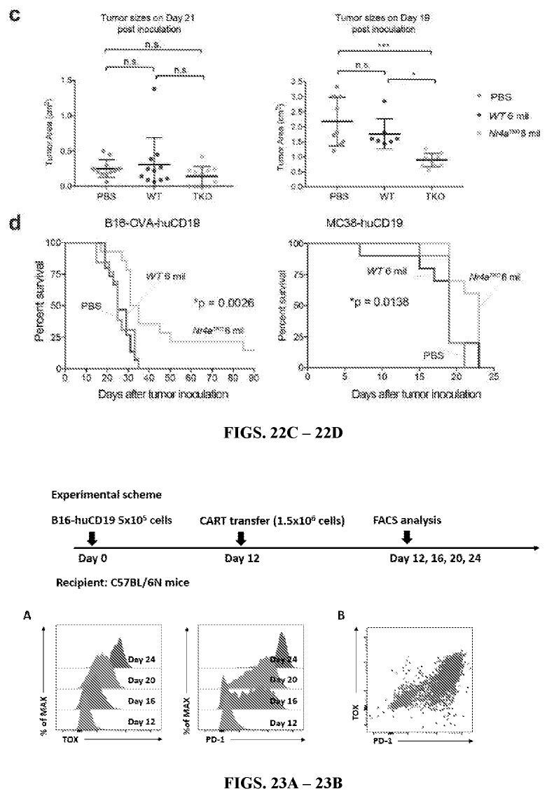

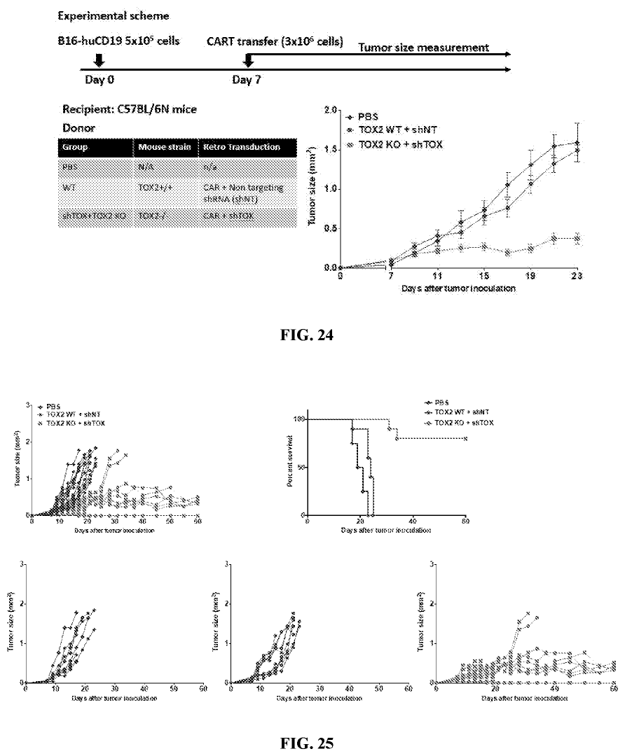

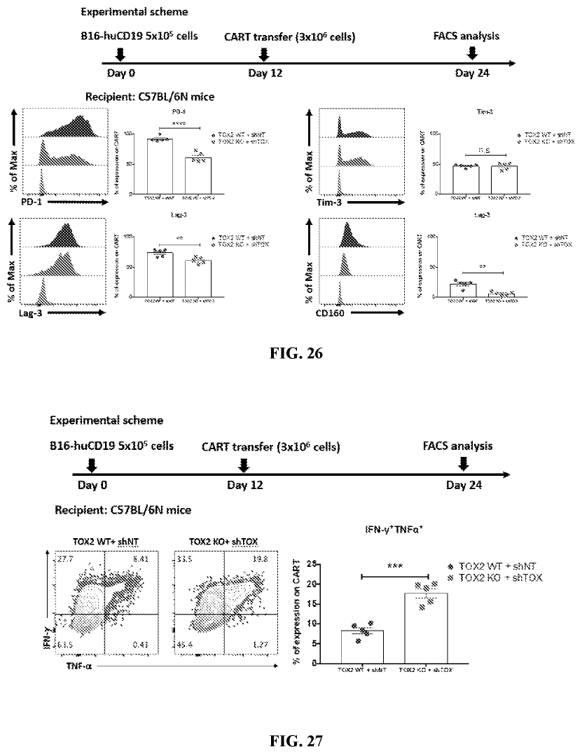

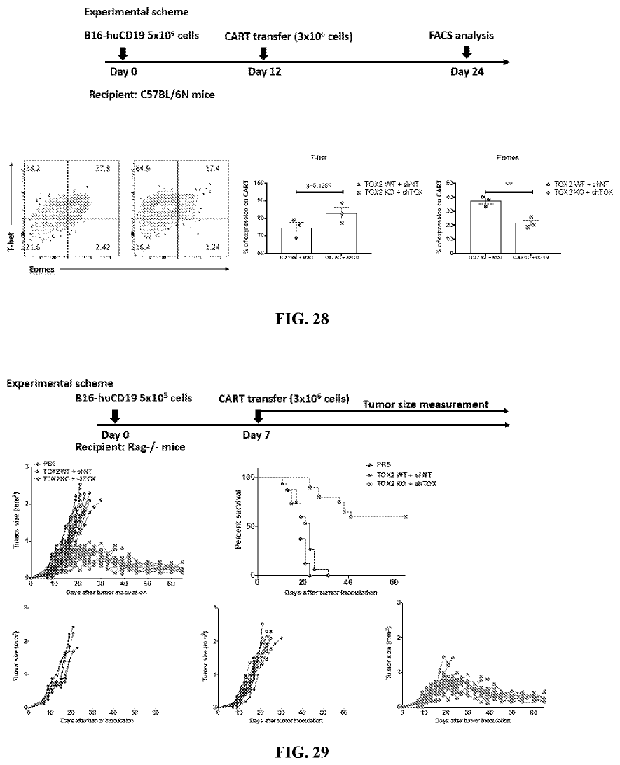

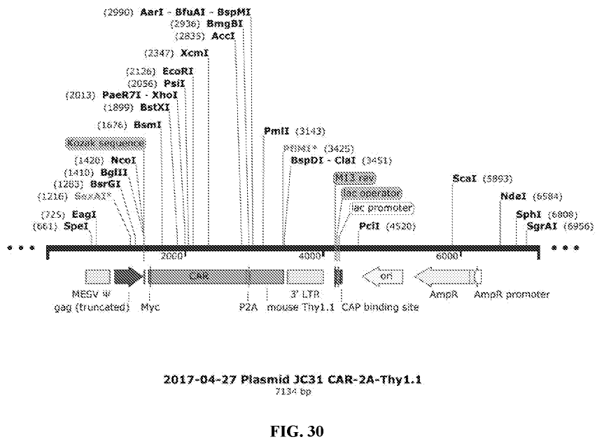

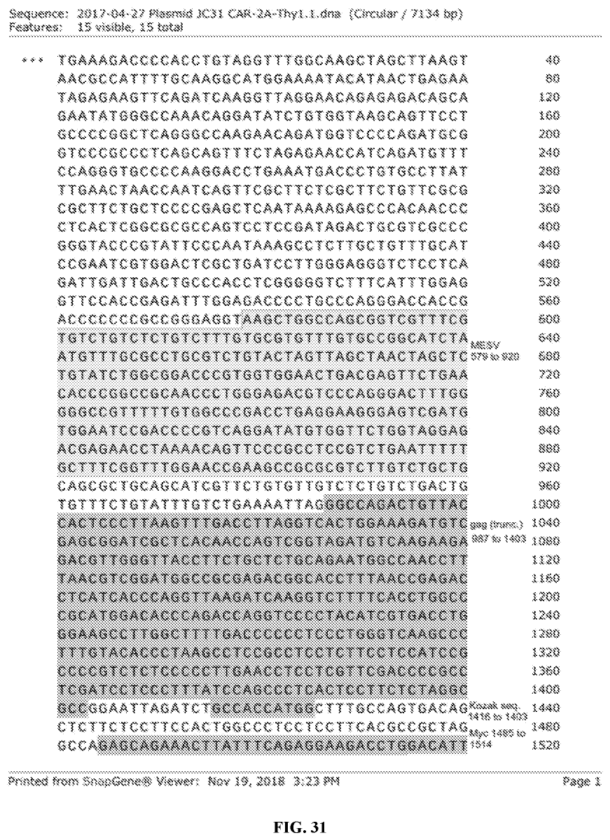

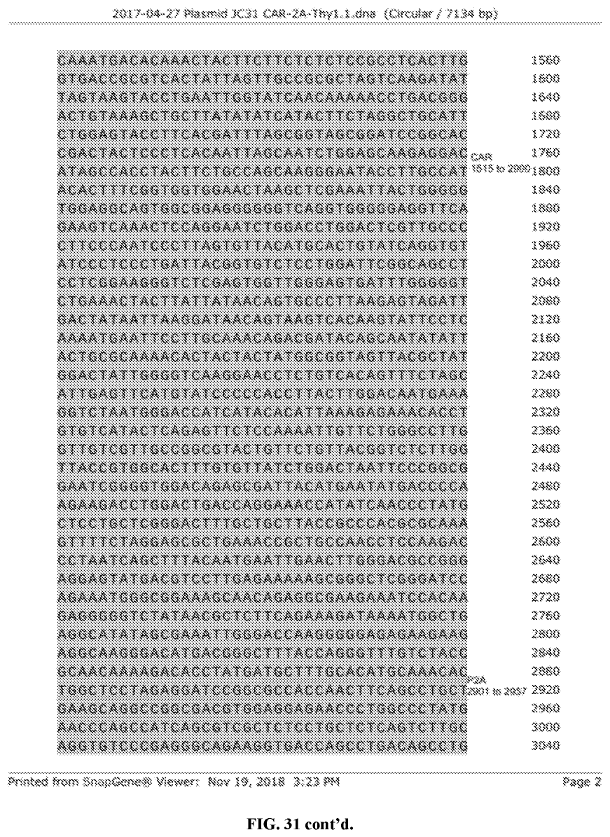

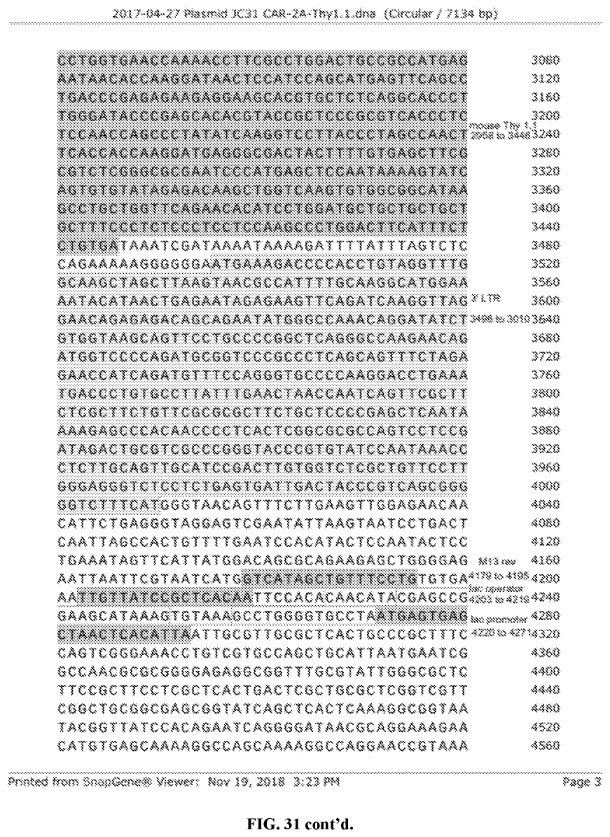

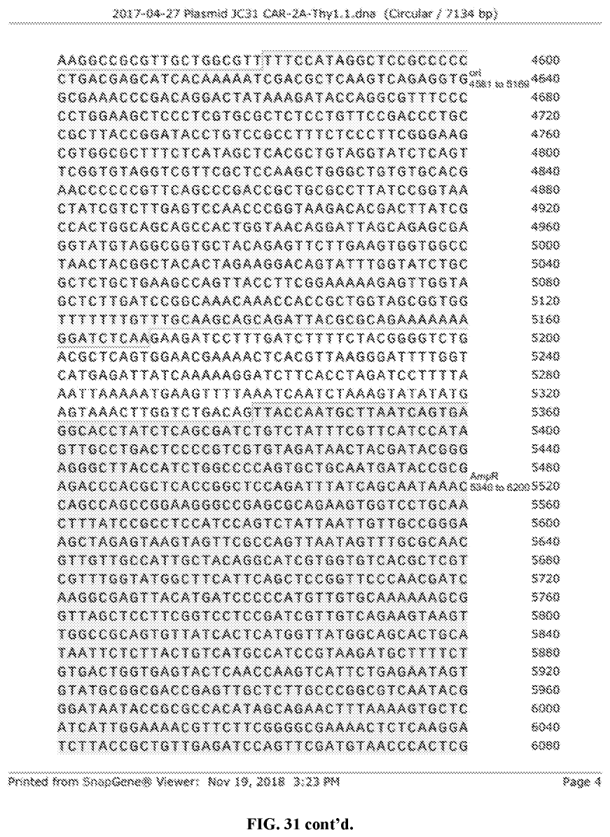

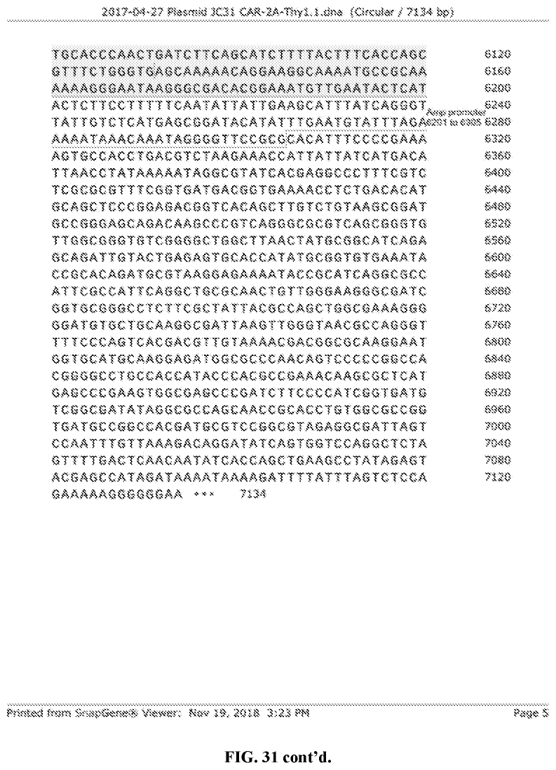

To determine whether differentially accessible regions enriched for Nr4a motifs in fact bound Nr4a, ectopically expressed individual HA-tagged Nr4a proteins in CD8+ T cells were used, and confirmed Nr4a binding by chromatin immunoprecipitation using the anti-HA antibody followed by qPCR for selected differentially accessible regions in gene loci that were differentially expressed. For instance, Ccr7, a gene whose expression is high in naïve and memory T cells and decreased in effector cells³⁷, is less expressed in effector-like Nr4a TKO compared to WT (more exhausted) TILs (FIG. 20A); consistent with lower expression, the distal 5′ region of Ccr7 has at least two ATAC-seq peaks that are less prominent in Nr4a TKO than in WT TILs and contain adjacent NFAT and Nr4a binding motifs (FIG. 20A, left panel, peach lines). ChIP-qPCR that ectopically expressed Nr4a bound these two Ccr7 enhancer regions (FIG. 20A, left panel, black lines, and right panels, bar plots). In contrast, the proximal promoter region and first intron of Ccr7 have ATAC-seq peaks that are more prominent in Nr4a TKO compared to WT TILs, and contain bZIP and NFκB motifs (FIG. 0.20, left panel, blue lines). Additional examples (Ifng, Ccr6) are shown in FIG. 19A (Ifng, Ccr6: left panels, genome browser views with NFAT and Nr4a motifs marked in peach and bZIP and NFkB motifs in blue; right panels, ChIP-qPCR for Nr4a binding to the indicated accessible regions). Examples of genes with enrichment of bZIP motifs in differentially accessible regions include Il21, which encodes a cytokine involved in effector function³⁸ and is more highly expressed in Nr4a TKO compared to WT TILs (FIG. 10A); two regions of the Il21 promoter gain accessibility in Nr4a TKO compared to WT TILs, one of which contains a bZIP motif (FIG. 20B). Similarly, the cytokine TNF is more highly expressed at both mRNA (FIG. 10A) and protein (FIG. 9G) levels in Nr4a TKO TILs, and the Tnf locus shows broadly increased accessibility across the promoter and the entire gene in Nr4a TKO TILs (FIG. 20B).

Inspection of chromatin accessibility at the Pdcd1 locus (encoding PD-1) shows that each of the Nr4a family members is at least partly responsible for increased accessibility of an enhancer located at ˜23 kb 5′ of the Pdcd1 transcription start site (FIG. 10C, gray box), which has been noted in all mouse models of exhaustion or dysfunction investigated so far^{17-19,21,29}. The ATAC-seq peak marking this enhancer is diminished in Nr4a TKO compared to WT CAR T cells, and increased in T cells ectopically expressing Nr4a1, Nr4a2 or Nr4a3 compared to cells transduced with empty vector alone. ChIP-qPCR shows that all three Nr4a family members bind at this enhancer region of the PD-1 locus (FIG. 10C, right). Notably, the small decrease in PD-1 MFI observed in the Nr4a TKO TILs is supported by a previous publication²¹ showing that deletion of this ˜23 kb enhancer region results in a small decrease in the mean fluorescence intensity of PD-1 staining in the EL-4 thymoma cell line.

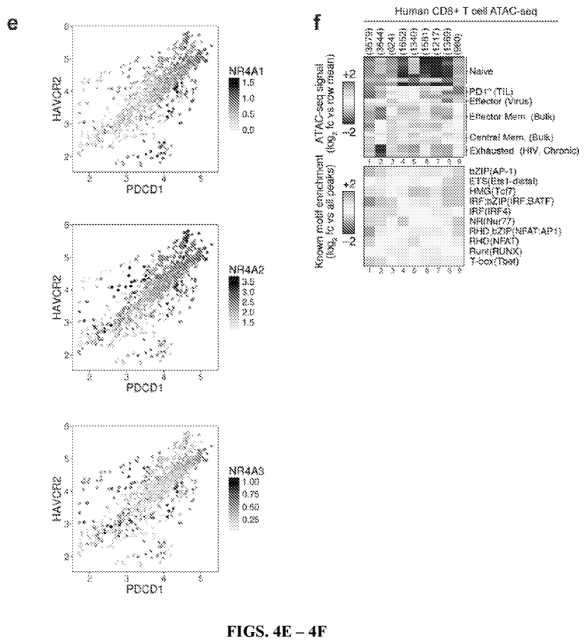

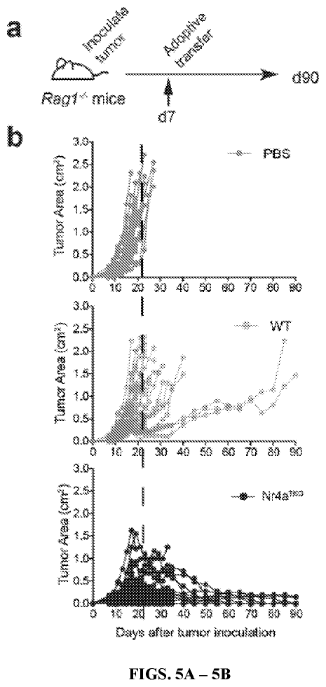

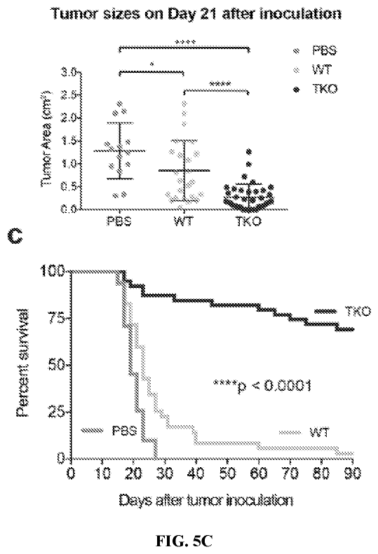

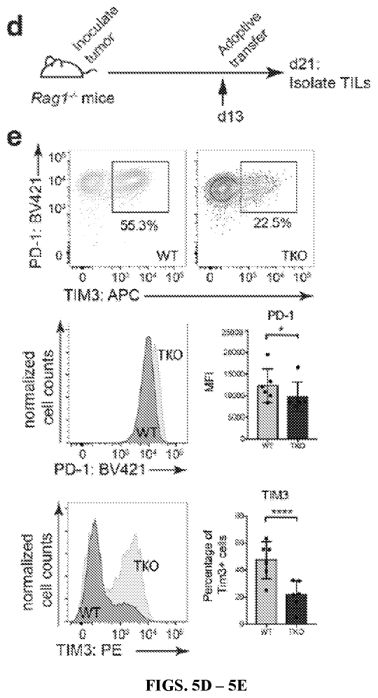

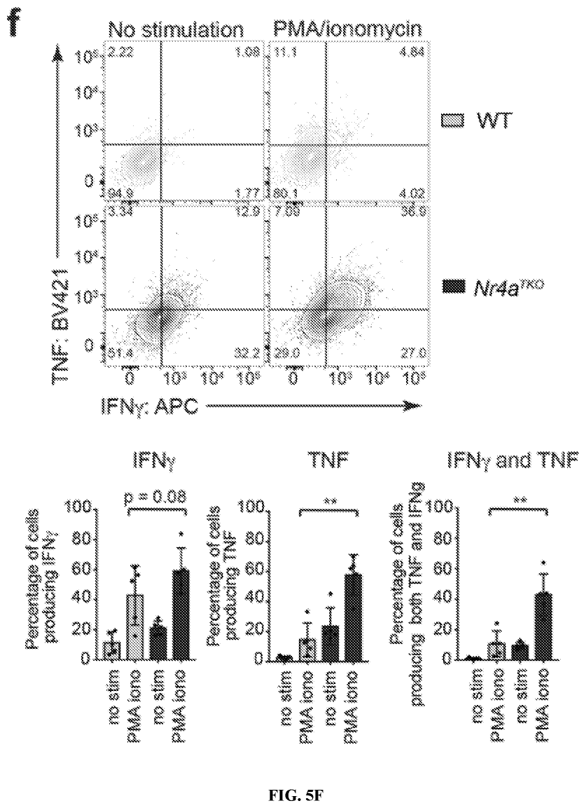

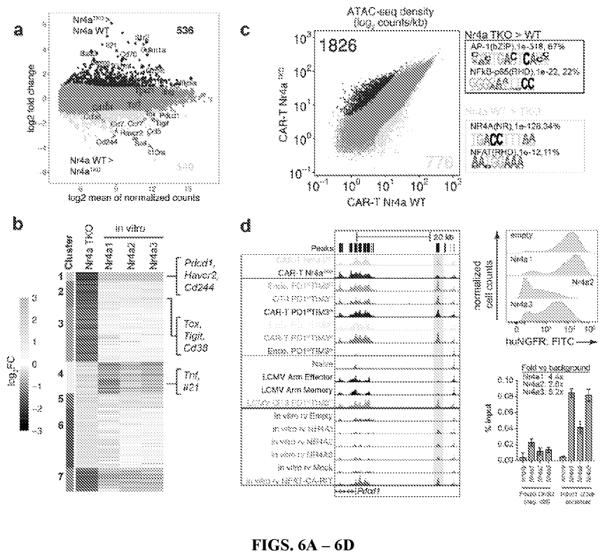

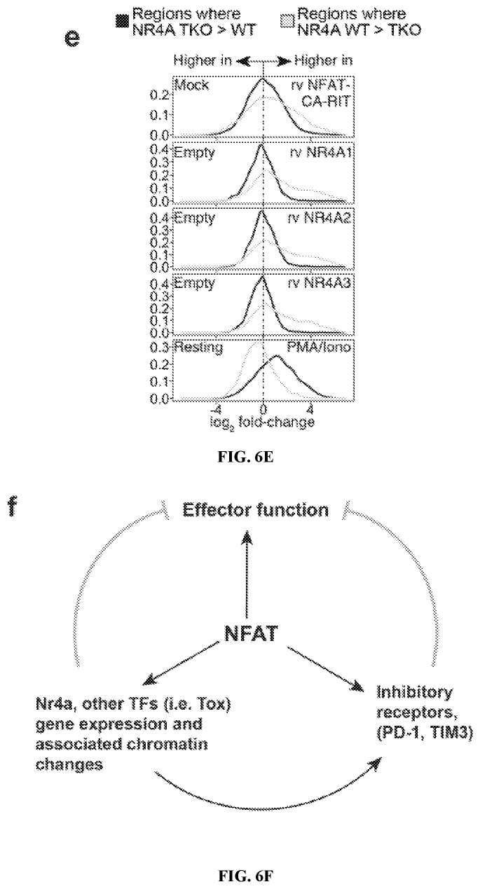

An engineered NFAT protein, CA-RIT-NFAT1, was used to mimic a dephosphorylated nuclear NFAT that cannot form cooperative transcriptional complexes with AP-1 (Fos-Jun)¹⁶. Compared to mock-treated cells, CA-RIT-NFAT1-transduced cells express higher levels of Nr4a transcription factors as well as inhibitory receptors, and display a transcriptional program that mimics in vivo exhaustion, particularly the early stages of “dysfunction”¹⁶⁻¹⁸. Genome-wide analysis of ATAC-seq data showed that regions that were more accessible in WT compared to Nr4a TKO TILs were also more accessible in cultured cells expressing CA-RIT-NFAT1 compared to mock-transduced cells, as well as in Nr4a-expressing compared to empty vector transduced cells (FIG. 4E). Thus, the regulatory elements sensitive to in vivo reduction of NR4A activity are sensitive to in vitro induction of constitutive NFAT and NR4A activity. Conversely, regions that were more accessible in Nr4a TKO TILs compared to WT TILs were more accessible in PMA/ionomycin stimulated cells compared to that of resting cells (FIG. 10C). As PMA/ionomycin stimulation engages bZIP and NFκB family member activity, these data indicate that the increased in vivo effector function and gene expression of Nr4a TKO TILs compared to WT TILs is associated with increased activity of these transcription factors.

Nr4a transcription factors are prominent, redundant effectors of the CD8⁺ T cell hyporesponsive program downstream of NFAT (FIG. 10C). Ectopic expression of each individual Nr4a protein represses cytokine function; conversely, TILs lacking all three Nr4a proteins display a gene expression profile characteristic of effector function, including increased expression of granzymes and cytokines. Nr4a TKO TILs also show increased chromatin accessibility at regions containing binding sites for transcription factors of the bZIP and Rel/NFκB families, which are involved in the classical program of T cell activation. Thus lack of Nr4a results in a permissive genomic landscape for T cell activation to occur. In addition, Nr4aTKO CAR TILs exhibit an increased frequency of a TIM3⁻ TCF1⁻ population (FIG. 12D, bottom) that may exhibit increased effector function, and is different from the TIM3⁻ TCF1+ memory/precursor population³⁹⁻⁴² that expands after PD-1 blockade³⁹ but is less represented in Nr4aTKO than in WT CAR TILs.

There is a clear relationship between PD-1 and Nr4a, both in our own studies and in previously published data from other labs^{17-19,21,29.} Ectopic expression of any individual Nr4a family member in vitro results in upregulation of PD-1 at both the protein and mRNA levels, as well as in increased accessibility at the −23 kb upstream enhancer region of the Pdcd1 locus. Nr4aTKO CAR TILs exhibit decreased accessibility at this enhancer compared to WT CAR TILs, and all three Nr4a family members bound this enhancer in cells. Taken together, these data indicate that the effect of triple Nr4a deficiency is functionally somewhat similar to PD-1 blockade (FIG. 10C), but Nr4a deletion has a broader effect than PD-1 blockade alone, by affecting a wide range of regulatory elements. In other words, PD-1 is likely to be only one of many genes regulated by the NFAT/Nr4a axis in both mouse and human T cells—the others encode other inhibitory receptors including CTLA4, TIM3, LAG3 and TIGIT.

Immune cell therapies offer considerable promise for the treatment of cancer^{43,44}. In some cases, endogenous CD8⁺ T cells that do not reject tumors efficiently can be rendered functional by antibodies that block inhibitory receptors such as PD-1 and CTLA4⁴⁵⁻⁴⁷. However, treatment with individual blocking antibodies rarely achieves complete cures, and hence the field of cancer immunotherapy is moving towards treatment with blocking antibodies to multiple inhibitory receptors. The NFAT/Nr4a axis controls the expression of multiple inhibitory receptors, and functionally, treatment of tumor-bearing mice with CAR T cells lacking all three Nr4a transcription factors resulted in tumor regression and prolonged survival. Thus inhibiting the function of Nr4a family members in tumor-infiltrating T cells could be a promising strategy for cancer immunotherapy.

### Materials and Methods

### Construction of Retroviral Vector (MSCV-myc-CAR-2A-Thy1.1) Containing Chimeric Antigen Receptor (CAR).

The chimeric antigen receptor was pieced together using published portions of the clone FMC63 human CD19 single chain variable fragment^{22,23}, and the published portions of the murine CD28 and CD3ζ sequences¹. The sequence for the myc tag on the N-terminus was obtained from published work⁴⁸. This chimeric antigen construct was then cloned into an MSCV-puro (Clontech) murine retroviral vector in place of the PGK-puro.

Polynucleotide sequence of CAR construct is provided in SEQ ID NO:1.

Amino acid sequence of CAR construct is provided in SEQ ID NO:2.

Construction of Retroviral Vector Containing huCD19.

DNA fragment encoding huCD19 was PCR-amplified and cloned into an MSCV-puro (Clontech) murine retroviral vector.

Construction of retroviral vectors containing Cre (MSCV-Cre-IRES-NGFR), and Nr4a1, Nr4a2, Nr4a3 (MCSV-HA-Nr4a1-IRES-NGFR, MCSV-HA-Nr4a2-IRES-NGFR, MCSV-HA-Nr4a3-IRES-NGFR).

DNA fragment encoding Cre was PCR-amplified and cloned into MSCV-IRES-NGFR (Addgene Plasmid #27489). DNA fragment encoding Nr4a1 (a kind gift of C.-W. J. Lio, La Jolla Institute for Allergy and Immunology, La Jolla, Calif.) was PCR-amplified with 5′ HA-tag and cloned into MSCV-IRES-NGFR. DNA fragment encoding Nr4a2 (Addgene Plasmid #3500) was PCR-amplified with 5′ HA-tag and cloned into MSCV-IRES-NGFR. DNA fragment encoding Nr4a3 (DNASU Plasmid #MmCD00080978) was PCR-amplified with 5′ HA-tag and cloned into MSCV-IRES-NGFR.

### Eukaryotic Cell Lines.

The EL4 mouse thymoma cell line was purchased from the American Type Culture Collection (ATCC): EL4 (ATCC® TIB-39™, Mus musculus T cell lymphoma). The B16-OVA mouse melanoma cell line expressing the ovalbumin protein (a kind gift of S. Schoenberger, La Jolla Institute for Allergy and Immunology, La Jolla, Calif.) was previously described¹⁸. The 293T cell line was purchased from ATCC: 293T (ATCC® CRL-3216™). The Platinum-E Retroviral Packaging Cell Line, Ecotropic (PlatE) cell line was purchased from Cell BioLabs, Inc: RV-101. The MC-38 mouse colon adenocarcinoma cell line (a kind gift of A. W. Goldrath, UCSD, La Jolla, Calif.) was originally purchased from Kerafast, Inc (ENH204).

Construction of Mouse Tumor Cell Lines Expressing huCD19.

B16-OVA, EL4, and MC-38 cells were transduced with an amphotropic virus containing the human CD19 (huCD19) and then sorted for cells expressing high levels of huCD19.

### Preparation of B16-OVA-huCD19 Melanoma Cells for Tumor Inoculation.

B16-OVA-huCD19 cells were cultured in Dulbecco's medium (DMEM) with 10% (vol/vol) FBS, 1% L-glutamine, 1% penicillin/streptomycin and passaged three times prior to inoculation. At the time of injection, cells were trypsinized and resuspended in Hanks balanced salt solution without phenol red at 10 million cells per milliliter. C57BL/6J male mice (8-12 wk old) were injected intradermally with 500,000 B16-OVA-huCD19 cells (50 μL per injection).

### Preparation of MC38-huCD19 Colon Adenocarcinoma Cells for Tumor Inoculation.

MC38-huCD19 cells were cultured in Dulbecco's medium (DMEM) with 10% (vol/vol) FBS, 0.1 mM non-essential amino acids, 1 mM sodium pyruvate, 10 mM Hepes, 1% L-glutamine, 1% penicillin/streptomycin and passaged two times prior to inoculation. At the time of injection, cells were trypsinized and resuspended in Hanks balanced salt solution without phenol red at 10 million cells per milliliter. C57BL/6J male mice (8-12 wk old) were injected intradermally with 500,000 MC38-huCD19 cells (50 μL per injection).

### Mice.

C57BL/6J, B6.SJL-Ptprc^{a}Pepc^{b}/BoyJ, Rag 1−/− mice were obtained from Jackson Laboratories. Nr4a gene-disrupted strains were obtained from Takashi Sekiya and Akihiko Yoshimura, with permission from Pierre Chambon. Both male and female mice were used for studies. Mice were age-matched and between 8-12 weeks old when used for experiments, and tumor-bearing mice were first tumor size-matched and then randomly assigned to experimental groups. All mice were bred and/or maintained in the animal facility at the La Jolla Institute for Allergy and Immunology. All experiments were performed in compliance with the LJI Institutional Animal Care and Use Committee (IACUC) regulations.

### B16-OVA-huCD19 Tumor Model.

For analysis of CAR CD8⁺ TILS and endogenous CD8⁺ TILs: On Day 0, 8-12 week old C57BL/6J mice were injected intradermally with 5×10⁵B16-OVA-huCD19 cells. After tumors became palpable, tumor measurements were recorded with a manual caliper every other day and tumor area was calculated in centimeters squared (length×width). On Day 13, 1.5 million CAR transduced CD45.1⁺ CD8⁺ T cells were adoptively transferred into tumor size-matched tumor-bearing mice. On Day 21, mice were harvested for tumors and spleens. For analysis of CAR CD8⁺ TILS lacking Nr4a family members: On Day 0, 8-12 week old Rag1−/− mice were injected intradermally with 5×10⁵B16-OVA-huCD19 cells and tumors were measured every other day after they became palpable. On Day 13, 1.5 million CAR- and empty vector pMIN-transduced Nr4a fl/fl Nr4a2 fl/fl Nr4a3+/+ or CAR- and Cre-transduced CD8⁺ Thy1.1⁺ NGFR⁺ Nr4a fl/fl Nr4a2 fl/fl Nr4a3−/− T cells were adoptively transferred into tumor size-matched tumor-bearing mice. On Day 21, mice were harvested for tumors and spleens. For monitoring of tumor growth for survival studies after adoptive transfer of CAR T cells lacking Nr4a family members: On Day 0, 8-12 week old Rag1−/− mice were injected intradermally with 5×10⁵B16-OVA-huCD19 cells and tumors were measured every other day after they became palpable. On Day 7, 3 million CAR- and empty vector pMIN-transduced or CAR- and Cre-transduced CD8⁺ Thy1.1⁺ NGFR⁺ Nr4a-floxed mouse T cells (in combinations to produce Nr4a1 KO, Nr4a2 KO, Nr4a3 KO, Nr4a TKO, WT as listed in FIG. 8) were adoptively transferred into tumor size-matched tumor-bearing mice. Tumor growth was then monitored until experimental endpoint on Day 90 after tumor inoculation or until IACUC endpoint.

### Preparation of Cells for Adoptive Transfer.

CD8⁺ T cells were isolated and activated with 1 ug/mL anti-CD3 and 1 ug/mL anti-CD28 for 1 d, then removed from activation and transduced with retrovirus expressing CAR, Cre, pMIN, or a combination of the above for 1 h at 37° C. and 2000 g. Immediately after the transduction, cells were replaced with media containing 100 U of IL-2/mL. 1 d following the first transduction, a second transduction was performed and immediately after the transduction, cells were replaced with media containing 100 U of IL-2/mL. On the day of adoptive transfer (either day 3 or day 5 post activation), cells were analyzed by flow cytometry and cell counts were obtained using a hemocytometer. The number of CAR-transduced cells was obtained using the cell counts from the hemocytometer and the population percentages obtained from flow cytometry. Cells were then collected, washed with PBS and resuspended at a concentration equivalent of 1.5 million or 3 million CAR-transduced cells per 200 uL of PBS. Mice were then adoptively transferred with 200 uL of retro-orbital i.v. injections each.

### Isolation of Tumor Infiltrating Lymphocytes (TILs) for Subsequent Analyses.

Sample preparation for flow cytometry of TILs from CAR and OT-I experiments: On Day 21, mice were euthanized and perfused with PBS prior to removal of tumor. Tumors were collected, pooled together by group, homogenized, and then dissociated using the MACS Miltenyi Mouse Tumor Dissociation kit (Miltenyi Biotec) and the gentleMACs dissociator with Octo Heaters (Miltenyi Biotec) according to manufacturer's instructions. Tumors were then filtered through a 70 uM filter and spun down. Supernatant was aspirated and the tumors were resuspended in the equivalent of 4-5 grams of tumor per 5 mL of 1% FBS/PBS for CD8 positive isolation using the Dynabeads FlowComp Mouse CD8 isolation kit (Invitrogen). After positive isolation, cells were either divided into equal amount for staining and phenotyping with flow cytometry, or stained for cell sorting. Sample preparation for flow cytometry of TILs from Nr4a WT and Nr4a TKO experiments:

On Day 21, mice were euthanized and perfused with PBS prior to removal of tumor. Tumors were collected, pooled together by group, homogenized, and then dissociated using the MACS Miltenyi Mouse Tumor Dissociation kit (Miltenyi Biotec) and the gentleMACs dissociator with Octo Heaters (Miltenyi Biotec) according to manufacturer's instructions. Tumors were then filtered through a 70 uM filter and spun down. Supernatant was aspirated and the tumors were resuspended in 40% Percoll/RPMI and underlaid with 80% Percoll/PBS in 15 mL conical tubes to form an 80%/40% Percoll discontinuous density gradient. Samples were spun for 30 min at room temperature at 1363 g in a large benchtop centrifuge with a swinging bucket. TILs were collected from 80%/40% Percoll interface and further purified using CD90.2 Microbeads (Miltenyi Biotec) and magnetic separation. After positive isolation, cells were either divided into equal amount for staining and phenotyping with flow cytometry, or stained for cell sorting.

### Transfections.

Transfections were performed in 10 cm dish format, following manufacturer's instructions for the TransIT®-LT1 Transfection Reagent (Mirus Bio LLC) and using the pCL10A1 and pCL-Eco packaging vectors (the former for the huCD19 virus, and the latter for all other viruses produced).

### Retroviral Transduction.

Retroviral transductions were performed in 6-well plate format, using 3 mL of 0.45 uM filtered virus and 8 ug/mL of polybrene per well. Double transductions were performed using 1.5 mL of each virus for a total of 3 mL. Cells were spun at 2000 g for 1 h at 37° C. in a pre-warmed centrifuge. Immediately after the transduction, cells were replaced with media containing 100 U of IL-2/mL. A second transduction is performed the following day.

### Antibodies.

Fluorochrome-conjugated antibodies were purchased from Biolegend, BD Sciences, eBioscience, and Cell Signaling Technologies. Primary antibody used for chromatin-immunoprecipitation was purchased from Cell Signaling Technologies.

### Surface Marker Staining.

Cells were spun down and stained with 1:200 final concentration of antibodies in 50% of 2.4G2 (Fc block) and 50% of FACS Buffer (PBS+1% FBS, 2 mM EDTA) for 15 min.

### Cytokine Restimulation and Staining.

Prior to staining, cells were incubated in media containing 10 nM of PMA and 500 nM of ionomycin, and 1 ug/mL of Brefeldin A at 37° C. for 4 hours. After restimulation, cells were then stained for surface markers and with live/dead dye as described in the surface marker staining protocol above. Cells were then fixed with 4% paraformaldehyde for 30 min, permeabilized with 1×BD Perm/Wash (BD Biosciences) for 30 min, and then stained for cytokines at a final concentration of 1:200 in 1×BD Perm/Wash buffer. 1×BD Perm/Wash buffer was prepared according to manufacturer's instructions. All wash steps were performed with FACS Buffer (PBS+1% FBS, 2 mM EDTA).

### Tf Staining.

Cells were stained for surface markers and with live/dead dye as described in the surface marker staining protocol above. Cells were then fixed, permeabilized, and stained using the Foxp3/Transcription Factor Staining Kit (eBioscience) according to manufacturer's instructions. All transcription factor antibodies were used at 1:200 final Concentration. The Antibody for Ki67 was Used at 1:100 Final Concentration.

### Flow Cytometry Analysis.

All flow cytometry analysis was performed using the LSRFortessa (BD Biosciences) or the LSR-II (BD Biosciences). Flow data was analyzed using FlowJo v.10 (Tree Star, Inc). Relevant sample gating has been provided in figures.

### Statistical Analyses.

Statistical analyses on flow cytometric data and tumor growth data for experiments involving were performed using the appropriate statistical comparison, including paired or unpaired two-tailed t-tests with Welch's correction as needed, one-way ANOVA with multiple comparisons test (Tukey's or Dunnett's), or ordinary two-way ANOVA (Prism 7, GraphPad Software). Statistical analyses for survival curves were performed using the Log-rank (Mantel-Cox) test (Prism 7, GraphPad Software). A p value of <0.05 was considered statistically significant.

### In Vitro Killing Assay.

10,000 B16-OVA-huCD19 cells were plated in 100 uL of T cell media as a target cells (or media only for background) were added to each well in E-plate 96 (ACEA Biosciences Inc., San Diego, Calif.). Plate was placed in xCELLigence Real-Time Cell Analysis (RTCA) instrument (ACEA Biosciences Inc., San Diego, Calif.) after 30 minutes and incubated overnight. The following day, the plate was removed from xCELLigence RTCA machine and CD8⁺ CART cells were added in an additional 100 uL of T cell media as an effector cells for 30 minutes (for lysis positive control, 0.2% TritonX was used, for lysis negative control, only media was used). The plate is then placed back into the incubator, and data acquisition begins. 5 hours after, the Cell Index (CI) was obtained from each well. Percentage of specific lysis was calculated for each well as follows: % specific lysis=100−(CI^{each well}/(CI^{pos}−CI^{neg})*100. B16-OVA-huCD19 cells were thawed out 3 days prior to plating on day 4 (when inoculation would usually occur); mouse CD8⁺ CAR T cells were prepared prior to the experiment to be added to target cells on day 5 post activation of CD8⁺ T cells.

### Chromatin Immunoprecipitation and Quantitative Real-Time PCR (ChIP-qPCR).

ChIP was performed as previously described⁴⁹. Briefly, CD8⁺ T cells were isolated from C57BL/6J mice as above, activated with plate-bound anti-CD3/CD28, transduced with either empty vector control or retrovirus expressing Nr4a1, Nr4a2, or Nr4a3 with hemagglutinin(HA)-tag on the N-terminus. Cells were cultured for a total of 5 days post-transduction. For fixation, formaldehyde (16%, ThermoFisher) was added directly to the cells to a final concentration of 1% and incubated at room temperature for 10 mins with constant agitation. Glycine (final 125 mM) was added to quench the fixation and the cells were washed twice with ice-cold PBS. Cell pellets were snap-frozen with liquid nitrogen and stored at −80° C. until use. For nuclei isolation, cell pellets were thawed on ice and lysed with lysis buffer (50 mM HEPES pH 7.5, 140 mM NaCl, 1 mM EDTA, 10% glycerol, 0.5% NP40, 0.25% Triton-X100) supplemented with 1% Halt protease inhibitor (ThermoFisher) for 10 mins at 4° C. with constant rotation. Pellets were washed once with washing buffer (10 mM Tris-HCl pH 8.0, 200 mM NaCl, 1 mM EDTA, 0.5 mM EGTA, 1% Halt protease inhibitor) and twice with shearing buffer (10 mM Tris-HCl pH 8.0, 1 mM EDTA, 0.1% SDS, 1% Halt protease inhibitor). Nuclei were resuspended in 1 mL shearing buffer, transferred to 1 mL milliTUBE (Covaris, Woburn, Mass.), and sonicated with Covaris E220 using for 18 minutes (Duty Cycle 5%, intensity 140 Watts, cycles per burst 200). After sonication, insoluble debris was removed by centrifugation at 20,000×g for 10 mins at 4° C. The concentration of chromatin was quantified using Qubit DNA BR assay (ThermoFisher). For immunoprecipitation, 25 ug of chromatin was removed and mixed with equal volume of 2× Conversion buffer (10 mM Tris-HCl pH 7.5, 280 mM NaCl, 1 mM EDTA, 1 mM EGTA, 0.2% sodium deoxycholate, 0.2% Triton-X100, 1% Halt protease inhibitor) in a 2 mL low-binding tube (Eppendorf). Either 5% or 6% of input chromatin was saved as control. Chromatin was pre-cleared using 30 uL washed protein A magnetic dynabeads (ThermoFisher) for 1 h at 4° C. with constant rotation. Pre-cleared chromatin was transferred to new tube, added with 10 ug rabbit monoclonal anti-HA (C29F4, Cell Signaling Technology) and 30 uL washed protein A magnetic dynabeads, and incubated at 4° C. overnight with constant rotation. Bead-bound chromatin was washed twice with RIPA buffer (50 mM Tris-HCl pH 8.0, 250 mM LiCl, 1 mM EDTA, 1% sodium deoxycholate, 1% NP-40, 0.1% SDS), once with high salt washing buffer (50 mM Tris-HCl pH 8.0, 500 mM NaCl, 1 mM EDTA, 1% NP-40, 0.1% SDS), once with Lithium washing buffer (50 mM Tris-HCl pH 8.0, 150 mM NaCl, 1 mM EDTA, 1% sodium deoxycholate, 1% NP-40), and once with TE (10 mM Tris-HCl pH 8.0, 1 mM EDTA). All washes were incubated for 5 mins at 4° C. with constant rotation. Chromatin was eluted from beads by incubating with elution buffer (100 mM NaHCO₃, 1% SDS) at room temperature for 30 mins in the presence of 0.5 mg/mL of RNaseA (Qiagen). To de-crosslink protein and DNA, proteinase K (final 0.5 mg/mL) and NaCl (final 200 mM) were added to the recovered supernatant and incubated at 65° C. overnight with constant shaking (1000 rpm) in a ThermoMixer (Eppendorf). DNA was purified using Zymo ChIP DNA clean and concentration kit (Zymo Research) according to the manual from the manufacturer. Eluted DNA was analyzed by qPCR using Power SYBR Green PCR Master Mix (Roche) and StepOne Real Time PCR system (ThermoFisher). The signals from ChIP sample was normalized to those form the input and calculated as “percentage of input”.

ChIP qPCR primers (all coordinates are for mm10).

1) chrX:7584283-7584409 127 bp: Fp3-CNS2-qF (forward) CCCAACAGACAGTGCAGGAA, (reverse) Fp3-CNS2-qR TGGTGTGACTGTGTGATGCA. 2) chr1:94074907-94075062 156 bp: Pd1.4A_qF1 (forward) ACCTTTCCTGTGCCTACGTC, Pd1.4A_qR1 (reverse) TAAGAGTGGTGGTGGTTGGG. 3) chr11:99163437-99163632 196 bp: CCR7_E1_F1 (forward) GGCTCTACTGCCCTGTTGTC, CCR7_E1_R1 (reverse) AACACATCATTTTGCCGTGA. 4) chr11:99168432-99168614 183 bp: CCR7_E2_F1 (forward) GGACACAGACGGGTGAGTTT, CCR7_E2_R1 (reverse) GGCCTGTGTTCAAATGAGGT. 5) chr17:8196147-8196301 155 bp: CCR6_F1 (forward) GGCAGGATGTGGCTTTGTAT, CCR6_R1 (reverse) CCTGCATGTAGTGCTGACCA 6) chr10:118460432-118460610 179 bp: IfngE_F1 (forward) GCGCCTAGAAGTTCAGTGCT, IfngE_R1 (reverse) TTTGAGATGCAGCAGTTTGG.

### Cell Sorting.

Cell sorting was performed by the LJI Flow Cytometry Core, using the FACSAria-I, FACSAria-II, or FACSAria-Fusion (BD Biosciences). For ATAC-seq, 50,000 cells were sorted from the isolated CD8⁺ TILS, with the exception of the OT-I samples, for which 15,000-30,000 cells were sorted. In some cases, a second ATAC-seq technical replicate using 50,000 additional cells was prepared in parallel. For RNA-seq, 10,000 cells were sorted from the isolated CD8⁺ TILs. For the CAR and OT-I experiments, the populations sorted were as follows: CD8⁺ CD45.1⁺ Thy1.1⁺ PD-1^{high} TIM3^{high} CAR (population A), CD8⁺ CD45.1⁺ Thy1.1⁺ PD-1^{high} TIM3^{high} CAR (population B), CD8⁺ CD45.1⁻ Thy1.1⁻ PD-1^{high} TIM3^{high} endogenous cells (population C), CD8⁺ CD45.1⁻ Thy1.1⁻ PD-1^{high} TIM3^{low} endogenous cells (population D) and CD8⁺ CD45.1⁻ Thy1.1⁻ PD-1^{low} TIM3^{low} endogenous cells (population E), and CD8⁺ CD45.1⁺ PD-1^{high} TIM3^{high} OT-I (population F). For the Nr4a experiments, populations were sorted as follows: CD8⁺ Thy1.1⁺ NGFR⁺ Nr4a WT TILs and CD8⁺ Thy1.1⁺ NGFR⁺ Nr4a TKO TILs. For the experiments ectopically expressing Nr4a in in vitro, populations were sorted on a set level of NGFR⁺ expression.

### ATAC-Seq Sample and Library Preparation.

ATAC-seq samples were prepared as in²⁵ with minor modifications. Briefly, cells were sorted into 50% FBS/PBS, spun down, washed once with PBS, and then lysed. Transposition reaction was performed using Nextera enzyme (Illumina) and purified using the MinElute kit (Qiagen) prior to PCR amplification (KAPA Biosystems) with 10-12 cycles using barcoded primers and 2×50 cycle paired-end sequencing (Illumina).

### ATAC-Seq Analysis.

Sequencing reads in FASTQ format were generated from Illumina Basespace (for mouse datasets) or were from published data^{19,21}. Reads were mapped to mouse (mm10) or human (hg19) genomes using bowtie (version 1.0.0,⁵⁰ with parameters “-p 8-m 1--best--strata-X 2000-S--fr--chunkmbs 1024.” Unmapped reads were processed with trim_galore using parameters “--paired--nextera--length 37--stringency 3--three_prime_clip_R1 1--three_prime_clip_R2 1” before attempting to map again using the above parameters. These two bam files were merged and processed to remove reads mapping to the mitochondrial genome and duplicate reads (with picard MarkDuplicates). For mouse datasets, technical replicates were merged together into one single biological replicate at this point. For human datasets, samples with low coverage or which did not meet quality control metrics were excluded. For the one human sample with two technical replicates, these matched closely and number 1 was chosen for the analysis. Genomic coverage for individual replicates were computed on 10 bp windows with MEDIPS⁵¹ using full fragments captured by ATAC-seq and used to generate average coverage with the Java Genomics Toolkit⁵² for each group.

To identify peaks, the barn files containing unique, non-chrM reads were processed with samtools and awk using “‘{ if(sqrt(\$9*\$9)<100){print \$0}}’” to identify nucleosome free DNA fragments less than 100 nt in length. These subnucleosomal fragments were used to call peak summits for each replicate with MACS2 using parameters “--nomodel--keep-dup all--call-summits.” For peak calling, a q value cutoff of 0.0001 for mouse datasets and 0.01 for human datasets was used. The summits for each peak from all replicates were expanded to regions with a uniform size of 200 bp for mouse datasets and 300 bp for human datasets. These regions from all replicates were merged into one global set of peaks and were filtered to remove peaks on the Y chromosome or those that overlapped ENCODE blacklisted regions^{53,54}.

Summarize Overlaps were used to compute the number of transposase insertions overlapping each peak from all replicates²⁷. For differential coverage, raw ATAC-seq counts in each peak for all replicates of all samples were normalized between replicates using voom⁵⁵. Pairwise contrasts were performed with limma and differentially accessible regions were filtered based on an fdr adjusted p-value of less than 0.01 and an estimated fold-change of at least 4. ATAC-seq density (number of transposase insertion sites per kilobase per million mapped reads) per peak and accessible regions were defined as those with a mean of 5 normalized insertions per kilobase. HOMER⁵⁶ was used to identify motifs for transcription factor binding sites enriched in different groups of peaks.

### RNA-Seq Sample and Library Preparation.

Total RNA was extracted using the RNeasy Micro Kit (Qiagen). SMARTseq2 libraries were prepared as described²⁴. Briefly, purified RNA was hybridized to polyA to enrich for mRNA, and then mRNA underwent reverse transcription and template switching prior to an 18-cycle PCR pre-amplification step. PCR cleanup was then performed using AMPure XP beads (Beckman Coulter). Quality check of the cDNA library was performed using an Agilent high-sensitivity DNA chip, and 1 ng of input cDNA was further used for library preparation using the Nextera XT LibraryPrep kit (Illumina). Tagmented DNA was amplified with a 12-cycle PCR and again purified with AMPureXP beads. Library size distribution and yield were evaluated using the Agilent high-sensitivity DNA chip. Libraries were pooled at equimolar ratios and sequenced with the rapid run protocol on a HiSEq2500 (Illumina) with 50-nt single-end cycling.

### RNA-Seq Analysis.

Quality and adapter trimming was performed on raw RNA-seq reads using TrimGalore! v0.4.5 (http://www.bioinformatics.babraham.ac.uk/projects/trim_galore/) with default parameters, retaining reads with minimal length of 36 bp. Resulting single-end reads were aligned to mouse genome mm10 using STAR v2.5.3 a²⁶ with alignment parameter outFilterMismatchNmax 4. Technical replicates were merged. RNA-seq analysis was performed at the gene level, employing the transcript annotations of the mouse genome mm10. Reads aligning to annotated features were counted using the summarizeOverlaps function (mode=“Union”) of the Bioconductor package GenomicAlignments v1.10.1²⁷. The DESeq2 package v1.14.1²⁸ was used to normalize the raw counts and identify differentially expressed genes (FDR cutoff of p<0.1, unless otherwise specified). Genes with less than 10 reads total were pre-filtered in all comparisons as an initial step. Transformed values (rlog) were calculated within DESeq2 for data visualization.

### Single Cell RNA-Seq Analysis.

Data was obtained from a previously published study on the cellular ecosystem of human melanoma tumors²⁰. Briefly,²⁰ profiled by single cell RNA-seq (scRNA-seq) malignant and non-malignant cells (including immune, stromal, and endothelial cells). Normalized expression values (E_{i,j}=log₂(TPM_{i,j}/10+1), where TPM_{i,j} refers to transcripts per million (TPM) for gene i in cell j) were obtained from Gene Expression Omnibus (GSE72056). For the analysis, only genes with non-zero expression values in at least 10 cells were kept. Given the technical noisiness and gene dropout associated with scRNA-seq data, the MAGIC algorithm⁵⁷ was used for imputation in the matrix of normalized expression values, with diffusion parameter t=2. An R implementation of the MAGIC method was downloaded from (https://www.krishnaswamylab.org/magic-project). Tumor infiltrating T cells were selected based on the inferred cell type annotation described in²⁰. CD8⁺ T cells were selected based on the expression of CD8A (cells with imputed values ≥4) and CD4 (cells with imputed values ≤1.5). Imputed values were used for gene expression visualizations.

### Gene Set Enrichment Analyses (GSEAs).

Gene set enrichment analysis (GSEA) was performed employing the GSEA Preranked function³², ranking genes by log 2 fold change according to the pertinent comparison, with number of permutations of 10,000 and allowing for gene set size up to 2000 genes. Gene sets were defined from differentially expressed genes obtained from pairwise comparisons between effector, memory, and exhausted CD8⁺ T cells from a previously published study¹⁷. In this context, differential gene expression was identified employing DESeq2 with FDR cutoff of p<0.01 and log 2 fold change cutoff of 1.

### Data Reporting.

No statistical methods were used to predetermine sample size. The investigators were not blinded to group allocation during experiments and outcome assessment.

### Data Availability.

All data generated and supporting the findings of his study are available within the paper. RNA-seq and ATAC-seq data have been deposited in the Gene Expression Omnibus (GEO) and accession codes will be available upon publication. Additional information and materials will be made available upon request.

### Material and Methods

### Mice.

C57BL/6N mice were purchased from Charles river Laboratories. Tox 2 gene KO strain were obtained from Dr. Avinash Bhandool (NIH, Baltimore, Md.). Rag 1−/− mice were obtained from Jackson Laboratories. Both male and female mice were used for studies. Mice were age-matched and between 8-12 weeks when used for experiments, and tumor bearing mice were first tumor size-matched and then randomly assigned to experimental groups. All mice were bred and maintained in the animal facility at the La Jolla institute for Immunology. All experiments were performed in compliance with the LJI Institutional Animal Care and Use Committee (IACUC) regulation.

### Preparation of CAR-T Cells.

CD8 T cells were isolated and activated with 1 ug/ml anti-CD3 and 1 ug/m anti-CD28 for 1 d, then removed from activation and transduced with retrovirus expressing CAR, non-targeting shRNA, TOX targeting shRNA or combination of the above fort 1 h 2000 g 37° C. Immediately after the transduction, cells were replaced with media containing 100 U of IL-2/ml. 1 d following the first transduction, a second transduction was performed and immediately after the transduction, cells were replaced with media containing 100 U of IL-2/ml. On the day of adoptive transfer, cells were analyzed by flow cytometry and cell counts were obtained using a hemocytometer. The number of CAR-transduced cells were obtained using the cell counts from the hemocytomeyter and the population percentages obtained from flow cytometry.

### B16-OVA-huCD19 Tumor Model.

For analysis of CAR CD8+TIL: On Day0, 8-12-week-old C57BL/6N mice were injected intradermally with 500K B16-OVA-huCD19 cells. After tumors became palpable, tumor measurements were recorded with a manual caliper every other day and tumor area was calculated in centimeter squared (Length×width). On Day 12, 3 million CAR- and non-targeting shRNA vector transduced TOX2+/+ T cells or CAR- and TOX targeting shRNA vector transduced TOX2−/− T cells were adoptively transferred into tumor size-matched tumor-bearing mice. On Day 24, mice were harvested for tumor. For monitoring of tumor growth for survival studies after adoptive transfer of CAR T cells lacking TOX family members: On Day0, 8-12-week-old C57BL/6N mice were injected intradermally with 500K B16-OVA-huCD19 cells. After tumors became palpable, tumor measurements were recorded with a manual caliper every other day and tumor area was calculated in centimeter squared (Length×width). On Day 12, 3 million CAR- and non-targeting shRNA vector transduced TOX2+/+ T cells or CAR- and TOX targeting shRNA vector transduced TOX2−/− T cells were adoptively transferred into tumor size-matched tumor-bearing mice. Tumor growth was then monitored until experimental endpoint after tumor inoculation or until IACUC endpoint.

### Cytokine Restimulation and Intra Cellular Staining.

For Cytokine: Prior to staining, cells were incubated in media containing 10 nM of PMA and 500 nM of ionomycin, and 1 ug/ml Brefeldin A at 37 degree for 4 hours. After restimulation, cells were then stained for surface markers and with live/dead dye as described in the surface marker staining protocol above. Cells were fixed with 4% paraformaldehyde for 30 min, permeabilized with 1×BD perm/Wash for 30 min and then stained for cytokine at a final concentration of 1:100 in 1×BD perm/wash buffer. 1×BD perm/wash buffer was prepared according to manufacturer's instruction. For transcription factor: Cells were stained for surface markers and with live/dead dye as described in the surface marker staining protocol above. Cells were then fixed, permeabilized, and stained using the Foxp3/Transcription Factor Staining Kit (eBioscience) according to manufacturer's instruction.

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

TABLE 1
Genes differentially expressed in Nr4a TKO relative to WT
CAR TILs. Genes differentially expressed (adjusted p value < 0.1
and log2Fold- Change ≥ 1 or ≤ −1) are highlighted.

| Genes | log2FoldChange | Pvalue_adjusted | Notes |
|---|---|---|---|
| Hapln1 | 6.108873287 | 3.04E−35 | Nr4a |
| | | | TKO >WT |
| Il23a | 5.325529984 | 4.92E−55 | Nr4a |
| | | | TKO >WT |
| Wisp1 | 5.05446419 | 2.32E−27 | Nr4a |
| | | | TKO > WT |
| Slc17a6 | 4.753982109 | 1.22E−63 | Nr4a |
| | | | TKO > WT |
| Il1r2 | 4.595827057 | 2.07E−47 | Nr4a |
| | | | TKO > WT |
| Ifitm1 | 4.562491119 | 2.03E−51 | Nr4a |
| | | | TKO > WT |
| Ifitm2 | 4.466715206 | 1.67E−69 | Nr4a |
| | | | TKO > WT |
| Csf2 | 4.389764071 | 8.37E−77 | Nr4a |
| | | | TKO > WT |
| Lrp1 | 4.38064071 | 5.23E−17 | Nr4a |
| | | | TKO > WT |
| Lta | 3.944000864 | 6.89E−59 | Nr4a |
| | | | TKO > WT |
| Il21 | 3.896908027 | 5.73E−19 | Nr4a |
| | | | TKO > WT |
| Trim66 | 3.875164559 | 2.89E−11 | Nr4a |
| | | | TKO > WT |
| 5830411N06Rik | 3.752496898 | 1.32E−10 | Nr4a |
| | | | TKO > WT |
| Ramp3 | 3.709785143 | 7.09E−13 | Nr4a |
| | | | TKO > WT |
| Areg | 3.63991464 | 2.00E−11 | Nr4a |
| | | | TKO > WT |
| Cdkn1a | 3.543671023 | 8.36E−48 | Nr4a |
| | | | TKO > WT |
| Clec12a | 3.424373 | 6.93E−21 | Nr4a |
| | | | TKO > WT |
| Cd70 | 3.235383968 | 2.75E−20 | Nr4a |
| | | | TKO > WT |
| Batf3 | 3.215155759 | 6.49E−08 | Nr4a |
| | | | TKO > WT |
| Ptprk | 3.191774717 | 9.40E−22 | Nr4a |
| | | | TKO > WT |
| Ccl1 | 3.12419465 | 9.43E−34 | Nr4a |
| | | | TKO > WT |
| Gzmf | 3.057601933 | 7.33E−10 | Nr4a |
| | | | TKO > WT |
| Tex15 | 3.020852142 | 1.47E−07 | Nr4a |
| | | | TKO > WT |
| P3h2 | 2.963994841 | 5.66E−07 | Nr4a |
| | | | TKO > WT |
| Tprg | 2.790686466 | 9.04E−06 | Nr4a |
| | | | TKO > WT |
| Siah2 | 2.788514824 | 6.23E−10 | Nr4a |
| | | | TKO > WT |
| Upp1 | 2.778556691 | 1.61E−06 | Nr4a |
| | | | TKO > WT |
| Rnf19b | 2.778410276 | 2.29E−16 | Nr4a |
| | | | TKO > WT |
| Gm156 | 2.719004729 | 3.80E−17 | Nr4a |
| | | | TKO > WT |
| Scimp | 2.70622405 | 3.52E−06 | Nr4a |
| | | | TKO > WT |
| Serpinb9b | 2.70126679 | 1.65E−22 | Nr4a |
| | | | TKO > WT |
| Mt2 | 2.658228685 | 1.56E−11 | Nr4a |
| | | | TKO > WT |
| Gzmc | 2.644263144 | 3.26E−19 | Nr4a |
| | | | TKO > WT |
| Cdkn2b | 2.629014182 | 1.11E−05 | Nr4a |
| | | | TKO > WT |
| Cxcl10 | 2.616233403 | 6.60E−16 | Nr4a |
| | | | TKO > WT |
| Cdh17 | 2.592217956 | 9.48E−06 | Nr4a |
| | | | TKO > WT |
| Hlf | 2.590332421 | 3.89E−05 | Nr4a |
| | | | TKO > WT |
| Socs2 | 2.587518259 | 4.06E−09 | Nr4a |
| | | | TKO > WT |
| Gzmg | 2.574512644 | 1.62E−07 | Nr4a |
| | | | TKO > WT |
| Vash1 | 2.55123118 | 1.40E−06 | Nr4a |
| | | | TKO > WT |
| Hbegf | 2.540913947 | 4.54E−08 | Nr4a |
| | | | TKO > WT |
| Mt1 | 2.520822254 | 1.45E−13 | Nr4a |
| | | | TKO > WT |
| Sema6d | 2.518825441 | 1.36E−07 | Nr4a |
| | | | TKO > WT |
| Tnfsf9 | 2.504129601 | 3.79E−21 | Nr4a |
| | | | TKO > WT |
| Dusp14 | 2.498839 | 9.00E−17 | Nr4a |
| | | | TKO > WT |
| Epha2 | 2.497335227 | 0.000148674 | Nr4a |
| | | | TKO > WT |
| Il2 | 2.49259591 | 2.56E−05 | Nr4a |
| | | | TKO > WT |
| Odc1 | 2.480370816 | 5.32E−20 | Nr4a |
| | | | TKO > WT |
| Fam20a | 2.455926078 | 3.19E−05 | Nr4a |
| | | | TKO > WT |
| Ebi3 | 2.432035641 | 4.06E−09 | Nr4a |
| | | | TKO > WT |
| Igfbp7 | 2.427459438 | 4.12E−08 | Nr4a |
| | | | TKO > WT |
| Msc | 2.398668532 | 2.30E−06 | Nr4a |
| | | | TKO > WT |
| Gadd45g | 2.386599001 | 8.56E−27 | Nr4a |
| | | | TKO > WT |
| Adora2b | 2.37081757 | 0.000266978 | Nr4a |
| | | | TKO > WT |
| Npnt | 2.348152919 | 0.000364677 | Nr4a |
| | | | TKO > WT |
| Serpinf1 | 2.347250236 | 3.64E−06 | Nr4a |
| | | | TKO > WT |
| Itga3 | 2.337797316 | 7.80E−08 | Nr4a |
| | | | TKO > WT |
| Capn5 | 2.331381784 | 4.90E−06 | Nr4a |
| | | | TKO > WT |
| Il1rl1 | 2.294161915 | 8.27E−12 | Nr4a |
| | | | TKO > WT |
| Gzme | 2.273980641 | 8.27E−07 | Nr4a |
| | | | TKO > WT |
| Mt3 | 2.268495298 | 6.90E−05 | Nr4a |
| | | | TKO > WT |
| Piwil2 | 2.256921971 | 0.00076918 | Nr4a |
| | | | TKO > WT |
| Rora | 2.200061327 | 8.61E−20 | Nr4a |
| | | | TKO > WT |
| Il2ra | 2.188897942 | 1.15E−08 | Nr4a |
| | | | TKO > WT |
| Abhd17b | 2.162121528 | 7.07E−05 | Nr4a |
| | | | TKO > WT |
| Gm15645 | 2.161202416 | 9.48E−06 | Nr4a |
| | | | TKO > WT |
| Lilrb4a | 2.155125192 | 1.94E−14 | Nr4a |
| | | | TKO > WT |
| Cenpv | 2.147098097 | 0.001161183 | Nr4a |
| | | | TKO > WT |
| Alyref | 2.139818846 | 6.20E−06 | Nr4a |
| | | | TKO > WT |
| Ifitm3 | 2.13556946 | 2.62E−12 | Nr4a |
| | | | TKO > WT |
| Marcksl1 | 2.119304519 | 0.000133708 | Nr4a |
| | | | TKO > WT |
| Lif | 2.110159766 | 0.000825135 | Nr4a |
| | | | TKO > WT |
| Pla2g12a | 2.106075955 | 0.000728641 | Nr4a |
| | | | TKO > WT |
| Adm | 2.105678204 | 0.002116964 | Nr4a |
| | | | TKO > WT |
| Gm16712 | 2.096188263 | 9.67E−05 | Nr4a |
| | | | TKO > WT |
| Krt17 | 2.09618043 | 0.001147567 | Nr4a |
| | | | TKO > WT |
| Zbtb46 | 2.075477124 | 0.002132034 | Nr4a |
| | | | TKO > WT |
| Reep2 | 2.06881887 | 0.000138885 | Nr4a |
| | | | TKO > WT |
| Nrn1 | 2.058647418 | 1.25E−19 | Nr4a |
| | | | TKO > WT |
| Mycn | 2.057669796 | 0.002068076 | Nr4a |
| | | | TKO > WT |
| Anxa1 | 2.057199825 | 4.69E−06 | Nr4a |
| | | | TKO > WT |
| Prkcdbp | 2.055206 | 0.002551645 | Nr4a |
| | | | TKO > WT |
| Rab34 | 2.054134868 | 0.002829457 | Nr4a |
| | | | TKO > WT |
| Epas1 | 2.049413922 | 9.01E−05 | Nr4a |
| | | | TKO > WT |
| Pdgfa | 2.02303643 | 0.003101547 | Nr4a |
| | | | TKO > WT |
| Ybx3 | 2.015598051 | 1.60E−15 | Nr4a |
| | | | TKO > WT |
| Lilr4b | 2.007890644 | 7.87E−11 | Nr4a |
| | | | TKO > WT |

TABLE 2
Genes differentially expressed in Nr4a TKO relative to WT
CAR TILs. Genes differentially expressed (adjusted p value < 0.1
and log2Fold- Change ≥ 1 or ≤ −1) are highlighted.

| Genes | log2Fold Change | Pvalue_adjusted | Notes |
|---|---|---|---|
| Ngfr | −5.002252842 | 1.27E−60 | *Used as |
| | | | marker |
| Slc6a12 | −4.705691896 | 4.15E−18 | Nr4a |
| | | | WT > TKO |
| Slc16a11 | −4.020665135 | 4.98E−12 | Nr4a |
| | | | WT > TKO |
| Il10ra | −3.826077207 | 1.11E−49 | Nr4a |
| | | | WT > TKO |
| Dgkh | −3.779518972 | 7.79E−11 | Nr4a |
| | | | WT > TKO |
| Gucy1a3 | −3.506211397 | 2.21E−09 | Nr4a |
| | | | WT > TKO |
| Mrc2 | −3.366653856 | 4.12E−08 | Nr4a |
| | | | WT > TKO |
| St6galnac3 | −3.306043176 | 8.11E−08 | Nr4a |
| | | | WT > TKO |
| Sell | −3.278164012 | 1.01E−18 | Nr4a |
| | | | WT > TKO |
| Havcr2 | −3.253909143 | 1.30E−29 | Nr4a |
| | | | WT > TKO |
| Filip1 | −3.060558254 | 9.49E−07 | Nr4a |
| | | | WT > TKO |
| Neb | −3.054273469 | 9.21E−07 | Nr4a |
| | | | WT > TKO |
| Eno3 | −3.007320839 | 1.05E−11 | Nr4a |
| | | | WT > TKO |
| Cd244 | −2.997654436 | 6.75E−17 | Nr4a |
| | | | WT > TKO |
| Penk | −2.973838536 | 2.07E−06 | Nr4a |
| | | | WT > TKO |
| Ccl5 | −2.922247753 | 1.36E−44 | Nr4a |
| | | | WT > TKO |
| Lyst | −2.776778703 | 2.21E−20 | Nr4a |
| | | | WT > TKO |
| Sdcbp2 | −2.718881172 | 8.29E−10 | Nr4a |
| | | | WT > TKO |
| Ildr1 | −2.694776546 | 7.98E−08 | Nr4a |
| | | | WT > TKO |
| Wls | −2.678017741 | 1.12E−08 | Nr4a |
| | | | WT > TKO |
| Cd7 | −2.670352489 | 1.56E−10 | Nr4a |
| | | | WT > TKO |
| Tbc1d4 | −2.61999889 | 2.86E−05 | Nr4a |
| | | | WT > TKO |
| Itih5 | −2.595139441 | 6.46E−12 | Nr4a |
| | | | WT > TKO |
| Hid1 | −2.476024866 | 2.00E−07 | Nr4a |
| | | | WT > TKO |
| 9930111J21Rik1 | −2.448565518 | 1.39E−11 | Nr4a |
| | | | WT > TKO |
| Insrr | −2.445344879 | 0.00021264 | Nr4a |
| | | | WT > TKO |
| Gm5431 | −2.430712581 | 2.27E−08 | Nr4a |
| | | | WT > TKO |
| Ifi44 | −2.420619518 | 8.65E−05 | Nr4a |
| | | | WT > TKO |
| Pde2a | −2.40515166 | 0.00028552 | Nr4a |
| | | | WT > TKO |
| Usp28 | −2.405054171 | 0.000279508 | Nr4a |
| | | | WT > TKO |
| Rap1gap2 | −2.404807879 | 0.00028552 | Nr4a |
| | | | WT > TKO |
| Pnpla7 | −2.384293555 | 9.39E−06 | Nr4a |
| | | | WT > TKO |
| Tor4a | −2.37197954 | 9.70E−12 | Nr4a |
| | | | WT > TKO |
| Kit | −2.352092252 | 0.000410029 | Nr4a |
| | | | WT > TKO |
| Cblb | −2.339264642 | 1.30E−19 | Nr4a |
| | | | WT > TKO |
| Clec16a | −2.33071958 | 1.40E−05 | Nr4a |
| | | | WT > TKO |
| Otub2 | −2.326753985 | 0.000314707 | Nr4a |
| | | | WT > TKO |
| Exoc3l | −2.304683813 | 1.40E−06 | Nr4a |
| | | | WT > TKO |
| BC147527 | −2.279324206 | 1.25E−05 | Nr4a |
| | | | WT > TKO |
| Khnyn | −2.259249976 | 5.74E−08 | Nr4a |
| | | | WT > TKO |
| Itga1 | −2.257808018 | 1.49E−05 | Nr4a |
| | | | WT > TKO |
| Fam65b | −2.244243009 | 9.37E−12 | Nr4a |
| | | | WT > TKO |
| Tsga10 | −2.237767508 | 0.000898918 | Nr4a |
| | | | WT > TKO |
| Tnfrsf1b | −2.227916895 | 4.33E−16 | Nr4a |
| | | | WT > TKO |
| Gm12185 | −2.224473307 | 2.03E−07 | Nr4a |
| | | | WT > TKO |
| Il6st | −2.19397954 | 5.02E−05 | Nr4a |
| | | | WT > TKO |
| Ccdc184 | −2.191777884 | 0.001178976 | Nr4a |
| | | | WT > TKO |
| Wdr95 | −2.179916723 | 0.000373848 | Nr4a |
| | | | WT > TKO |
| Tlr12 | −2.144020007 | 0.001499431 | Nr4a |
| | | | WT > TKO |
| Sidt1 | −2.113094246 | 9.13E−10 | Nr4a |
| | | | WT > TKO |
| Pydc4 | −2.103692595 | 3.84E−07 | Nr4a |
| | | | WT > TKO |
| Hs1bp3 | −2.099027844 | 0.002252352 | Nr4a |
| | | | WT > TKO |
| St8sia4 | −2.094598221 | 9.49E−07 | Nr4a |
| | | | WT > TKO |
| Pogk | −2.079997822 | 0.000369025 | Nr4a |
| | | | WT > TKO |
| Abi3 | −2.056253316 | 0.000988709 | Nr4a |
| | | | WT > TKO |
| Cd86 | −2.024373779 | 1.01E−06 | Nr4a |
| | | | WT > TKO |
| Pde4a | −2.024347335 | 0.001058763 | Nr4a |
| | | | WT > TKO |
| Foxred2 | −2.008191048 | 0.000806288 | Nr4a |
| | | | WT > TKO |
| Sgsh | −2.003114545 | 0.000178766 | Nr4a |
| | | | WT > TKO |
| Zfp862-ps | −2.002066474 | 0.003975931 | Nr4a |
| | | | WT > TKO |

### REFERENCES

1. Kochenderfer, J. N., Yu, Z., Frasheri, D., Restifo, N. P. & Rosenberg, S. A. Adoptive transfer of syngeneic T cells transduced with a chimeric antigen receptor that recognizes murine CD19 can eradicate lymphoma and normal B cells. Blood 116, 3875-3886 (2010).
2. Milone, M. C. et al. Chimeric receptors containing CD137 signal transduction domains mediate enhanced survival of T cells and increased antileukemic efficacy in vivo. Mol. Ther. 17, 1453-1464 (2009).
3. Davila, M. L. et al. Efficacy and toxicity management of 19-28z CAR T cell therapy in B cell acute lymphoblastic leukemia. Sci. Transl. Med. 6, (2014).
4. Maude, S. L. et al. Chimeric Antigen Receptor T Cells for Sustained Remissions in Leukemia. N. Engl. J. Med. 371, 1507-1517 (2014).
5. Ahmed, N. et al. Human Epidermal Growth Factor Receptor 2 (HER2)-Specific Chimeric Antigen Receptor—Modified T Cells for the Immunotherapy of HER2-Positive Sarcoma. J. Clin. Oncol. 33, 1688-1696 (2015).
6. O'Rourke, D. M. et al. A single dose of peripherally infused EGFRvIII-directed CAR T cells mediates antigen loss and induces adaptive resistance in patients with recurrent glioblastoma. Sci. Transl. Med. 9, (2017).
7. Pereira, R. M., Hogan, P. G., Rao, A. & Martinez, G. J. Transcriptional and epigenetic regulation of T cell hyporesponsiveness. J. Leukoc. Biol. jlb.2R10317-097R (2017). doi:10.1189/jlb.2R10317-097R
8. Wherry, E. J. T cell exhaustion. Nat. Immunol. 131, 492-499 (2011).
9. Wherry, E. J. et al. Molecular Signature of CD8+ T Cell Exhaustion during Chronic Viral Infection. Immunity 27, 670-684 (2007).
10. Blackburn, S. D. et al. Coregulation of CD8+ T cell exhaustion by multiple inhibitory receptors during chronic viral infection. Nat. Immunol. 10, 29-37 (2009).
11. Barber, D. L. et al. Restoring function in exhausted CD8 T cells during chronic viral infection. Nature 439, 682-687 (2006).
12. Schietinger, A. et al. Tumor-Specific T Cell Dysfunction Is a Dynamic Antigen-Driven Differentiation Program Initiated Early during Tumorigenesis. Immunity 45, (2016).
13. Schietinger, A. & Greenberg, P. D. Tolerance and exhaustion: Defining mechanisms of T cell dysfunction. Trends in Immunology 35, (2014).
14. Moon, E. K. et al. Multifactorial T cell Hypofunction That is Reversible Can Limit the Efficacy of Chimeric Antibody Receptor-transduced Human T cells in Solid Tumors. Clin. Cancer Res. 20, 4262-73 (2014).
15. John, L. B. et al. Anti-PD-1 antibody therapy potently enhances the eradication of established tumors by gene-modified T cells. Clin. Cancer Res. 19, 5636-5646 (2013).
16. Martinez, G. J. et al. The Transcription Factor NFAT Promotes Exhaustion of Activated CD8+ T Cells. Immunity 42, (2015).
17. Scott-Browne, J. P. et al. Dynamic Changes in Chromatin Accessibility Occur in CD8+ T Cells Responding to Viral Infection. Immunity 45, 1327-1340 (2016).
18. Mognol, G. P. et al. Exhaustion-associated regulatory regions in CD8⁺ tumor-infiltrating T cells. Proc. Natl. Acad. Sci. 201620498 (2017). doi:10.1073/pnas.1620498114
19. Philip, M. et al. Chromatin states define tumour-specific T cell dysfunction and reprogramming. Nature 545, 452 (2017).
20. Tirosh, I. et al. Dissecting the multicellular ecosystem of metastatic melanoma by single-cell RNA-seq. Science (80-.). (2016). doi:10.1126/science.aad0501
21. Sen, D. R. et al. The epigenetic landscape of T cell exhaustion. Science (80-.). 354, (2016).
22. Nicholson, I. C. et al. Construction and characterisation of a functional CD19 specific single chain Fv fragment for immunotherapy of B lineage leukaemia and lymphoma. Mol. Immunol. 34, 1157-1165 (1997).
23. Brogdon, J., June, C. H., Loew, A., Maus, M. & Scholler, J. Treatment of cancer using humanized anti-cd19 chimeric antigen receptor. (2014).
24. Picelli, S. et al. Full-length RNA-seq from single cells using Smart-seq2. Nat. Protoc. 9, 171-181 (2014).
25. Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods 10, (2013).
26. Dobin, A. et al. STAR: Ultrafast universal RNA-seq aligner. Bioinformatics 29, 15-21 (2013).
27. Lawrence, M. et al. Software for Computing and Annotating Genomic Ranges. PLoS Comput. Biol. 9, (2013).
28. Love, M. I., Huber, W. & Anders, S. Moderated estimation of fold change and dispersion for RNA-seq data with DESeq2. Genome Biol. 15, (2014).
29. Pauken, K. E. et al. Epigenetic stability of exhausted T cells limits durability of reinvigoration by PD-1 blockade. Science 354, (2016).
30. Sekiya, T. et al. Nr4a receptors are essential for thymic regulatory T cell development and immune homeostasis. Nat. Immunol. 14, 230-237 (2013).
31. Kadkhodaei, B. et al. Nurr1 Is Required for Maintenance of Maturing and Adult Midbrain Dopamine Neurons. J. Neurosci. (2009). doi:10.1523/JNEUROSCI.3910-09.2009
32. Subramanian, A. et al. Gene set enrichment analysis: A knowledge-based approach for interpreting genome-wide expression profiles. Proc. Natl. Acad. Sci. 102, 15545-15550 (2005).
33. Nowyhed, H. N. et al. The Nuclear Receptor Nr4a1 Controls CD8 T Cell Development Through Transcriptional Suppression of Runx3. Sci. Rep. 5, 9059 (2015).
34. Milner, J. J. et al. Runx3 programs CD8+ T cell residency in non-lymphoid tissues and tumours. Nature (2017). doi:10.1038/nature24993
35. Harant, H. & Lindley, I. J. D. Negative cross-talk between the human orphan nuclear receptor Nur77/NAK-1/TR3 and nuclear factor-KB. Nucleic Acids Res. (2004). doi:10.1093/nar/gkh856
36. Saijo, K. et al. A Nurr1/CoREST Pathway in Microglia and Astrocytes Protects Dopaminergic Neurons from Inflammation-Induced Death. Cell 137, 47-59 (2009).
37. Best, J. A. et al. Transcriptional insights into the CD8+ T cell response to infection and memory T cell formation. Nat. Immunol. 14, 404-412 (2013).
38. Spolski, R. & Leonard, W. J. Interleukin-21: Basic Biology and Implications for Cancer and Autoimmunity. Annu. Rev. Immunol. 26, 57-79 (2008).
39. Im, S. J. et al. Defining CD8+ T cells that provide the proliferative burst after PD-1 therapy. Nature (2016). doi:10.1038/nature19330
40. Leong, Y. A. et al. CXCRS+follicular cytotoxic T cells control viral infection in B cell follicles. Nat. Immunol. (2016). doi:10.1038/ni.3543
41. He, R. et al. Follicular CXCRS-expressing CD8+ T cells curtail chronic viral infection. Nature (2016). doi:10.1038/nature19317
42. Utzschneider, D. T. et al. T Cell Factor 1-Expressing Memory-like CD8+ T Cells Sustain the Immune Response to Chronic Viral Infections. Immunity (2016). doi:10.1016/j.immuni.2016.07.021
43. Jackson, H. J., Rafiq, S. & Brentjens, R. J. Driving CAR T-cells forward. Nat. Rev. Clin. Oncol. 13, 370-383 (2016).
44. Fesnak, A. D., June, C. H. & Levine, B. L. Engineered T cells: the promise and challenges of cancer immunotherapy. Nat. Rev. Cancer 16, 566-581 (2016).
45. Hodi, F. S. et al. Improved survival with ipilimumab in patients with metastatic melanoma. N Engl. J Med. 363, 711-23 (2010).
46. Ribas, A. et al. Association of Pembrolizumab With Tumor Response and Survival Among Patients With Advanced Melanoma. JAMA 315, 1600 (2016).
47. Larkin, J. et al. Combined Nivolumab and Ipilimumab or Monotherapy in Untreated Melanoma. N. Engl. J. Med. 373, 23-34 (2015).
48. Roybal, K. T. et al. Precision Tumor Recognition by T Cells with Combinatorial Antigen-Sensing Circuits. Cell 164, 770-779 (2016).
49. Lio, C. W. et al. Tet2 and Tet3 cooperate with B-lineage transcription factors to regulate DNA modification and chromatin accessibility. Elife 5, (2016).
50. Langmead, B., Trapnell, C., Pop, M. & Salzberg, S. Ultrafast and memory-efficient alignment of short DNA sequences to the human genome. Genome Biol. 10, R25 (2009).
51. Chavez, L. et al. Computational analysis of genome-wide DNA methylation during the differentiation of human embryonic stem cells along the endodermal lineage. Genome Res. 20, 1441-1450 (2010).
52. Palpant, T. Java Genomics Toolkit. (2016). Available at:
   https://github.com/timpalpant/java-genomics-toolkit.
53. Consortium, E. P. et al. An integrated encyclopedia of DNA elements in the human genome. Nature 489, 57-74 (2012).
54. Kundaje, A. Blacklisted genomic regions for functional genomics analysis. (2014). Available at: https://sites.google.com/site/anshulkundaje/projects/blacklists.
55. Ritchie, M. E. et al. limma powers differential expression analyses for RNA-sequencing and microarray studies. Nucleic Acids Res. 43, e47 (2015).
56. Heinz, S. et al. Simple Combinations of Lineage-Determining Transcription Factors Prime cis-Regulatory Elements Required for Macrophage and B Cell Identities. Mol. Cell 38, 576-589 (2010).
57. van Dijk, D. et al. Recovering Gene Interactions from Single-Cell Data Using Data Diffusion. Cell (2018). doi:10.1016/j.cell.2018.0cd85.061

Sequences
polynucleotide sequence of CAR construct:
SEQ ID NO: 1
GACATTCAAATGACACAAACTACTTCTTCTCTCTCCGCCTCACTTGGTGA
CCGCGTCACTATTAGTTGCCGCGCTAGTCAAGATATTAGTAAGTACCTGA
ATTGGTATCAACAAAAACCTGACGGGACTGTAAAGCTGCTTATATATCAT
ACTTCTAGGCTGCATTCTGGAGTACCTTCACGATTTAGCGGTAGCGGATC
CGGCACCGACTACTCCCTCACAATTAGCAATCTGGAGCAAGAGGACATAG
CCACCTACTTCTGCCAGCAAGGGAATACCTTGCCATACACTTTCGGTGGT
GGAACTAAGCTCGAAATTACTGGGGGTGGAGGCAGTGGCGGAGGGGGGTC
AGGTGGGGGAGGTTCAGAAGTCAAACTCCAGGAATCTGGACCTGGACTCG
TTGCCCCTTCCCAATCCCTTAGTGTTACATGCACTGTATCAGGTGTATCC
CTCCCTGATTACGGTGTCTCCTGGATTCGGCAGCCTCCTCGGAAGGGTCT
CGAGTGGTTGGGAGTGATTTGGGGGTCTGAAACTACTTATTATAACAGTG
CCCTTAAGAGTAGATTGACTATAATTAAGGATAACAGTAAGTCACAAGTA
TTCCTCAAAATGAATTCCTTGCAAACAGACGATACAGCAATATATTACTG
CGCAAAACACTACTACTATGGCGGTAGTTACGCTATGGACTATTGGGGTC
AAGGAACCTCTGTCACAGTTTCTAGCATTGAGTTCATGTATCCCCCACCT
TACTTGGACAATGAAAGGTCTAATGGGACCATCATACACATTAAAGAGAA
ACACCTGTGTCATACTCAGAGTTCTCCAAAATTGTTCTGGGCCTTGGTTG
TCGTTGCCGGCGTACTGTTCTGTTACGGTCTCTTGGTTACCGTGGCACTT
TGTGTTATCTGGACTAATTCCCGGCGGAATCGGGGTGGACAGAGCGATTA
CATGAATATGACCCCAAGAAGACCTGGACTGACCAGGAAACCATATCAAC
CCTATGCTCCTGCTCGGGACTTTGCTGCTTACCGCCCACGCGCAAAGTTT
TCTAGGAGCGCTGAAACCGCTGCCAACCTCCAAGACCCTAATCAGCTTTA
CAATGAATTGAACTTGGGACGCCGGGAGGAGTATGACGTCCTTGAGAAAA
AGCGGGCTCGGGATCCAGAAATGGGCGGAAAGCAACAGAGGCGAAGAAAT
CCACAAGAGGGGGTCTATAACGCTCTTCAGAAAGATAAAATGGCTGAGGC
ATATAGCGAAATTGGGACCAAGGGGGAGAGAAGAAGAGGCAAGGGACATG
ACGGGCTTTACCAGGGTTTGTCTACCGCAACAAAAGACACCTATGATGCT
TTGCACATGCAAACACTGGCTCCTAGAGGATCCGGC
Amino acid sequence of CAR construct:
SEQ ID NO: 2
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSIEFMYPPP
YLDNERSNGTIIHIKEKHLCHTQSSPKLFWALVVVAGVLFCYGLLVTVAL
CVIWTNSRRNRGGQSDYMNMTPRRPGLTRKPYQPYAPARDFAAYRPRAKF
SRSAETAANLQDPNQLYNELNLGRREEYDVLEKKRARDPEMGGKQQRRRN
PQEGVYNALQKDKMAEAYSEIGTKGERRRGKGHDGLYQGLSTATKDTYDA
LHMQTLAPRGSG
Homo sapiens NR4A1 polynucleotide sequence
SEQ ID NO: 3
>12 dna: chromosome chromosome: GRCh38: 12:
52022232: 52060107: 1
GenBank RefSeq (mRNA): NM_001202233;
NM_001202234; NM_002135; NM_173157; NM_173158
GenBank RefSeq (protein): NP_001189162;
NP_001189163; NP_002126; NP_775180; NP_002126.2
Homo sapiens NR4A2 polynucleotide sequence
SEQ ID NO: 4
>2 dna: chromosome chromosome: GRCh38: 2:
156323832: 156342948: -1
GenBank RefSeq (mRNA): NM_006186; NM_173171;
NM_173172; NM_173173
GenBank RefSeq (protein): NP_006177; NP_775265;
NP_006177.1
Homo sapiens NR4A3 polynucleotide sequence
SEQ ID NO: 5
>9 dna: chromosome chromosome: GRCh38: 9:
99821255: 99867491: 1
GenBank RefSeq (mRNA): NM_006981; NM_173198;
NM_173199; NM_173200
GenBank RefSeq (protein): NP_008912; NP_775291;
NP_775292
Homo sapiens TOX1 polynucleotide sequence
SEQ ID NO: 6
>8 dna: chromosome chromosome: GRCh38: 8:
58804818: 59119808: -1
GenBank RefSeq (mRNA): NM_014729
GenBank RefSeq (protein): NP_055544
Homo sapiens TOX2 polynucleotide sequence
SEQ ID NO: 7
>20 dna: chromosome chromosome: GRCh38: 20:
43914264: 44070216: 1
GenBank RefSeq (mRNA): NM_001098796.1;
NM_001098797.1; NM_001098798.1; NM_032883.2
GenBank RefSeq (protein): NP_001092266.1;
NP_001092267.1; NP_001092268.1; NP_ 116272.1
Homo sapiens TOX3 polynucleotide sequence
SEQ ID NO: 8
>16 dna: chromosome chromosome: GRCh38: 16:
52437405: 52548402: -1
GenBank RefSeq (mRNA): NM_001080430.3;
NM_001146188.2
GenBank RefSeq (protein): NP_001073899.2;
NP_001139660.1
Homo sapiens TOX4 polynucleotide sequence
SEQ ID NO: 9
>14 dna: chromosome chromosome: GRCh38: 14:
21475997: 21499775: 1
GenBank RefSeq (mRNA): NM_014828; NM_001303523
GenBank RefSeq (protein): NP_001290452; NP_055643
Homo sapiens IL-21 polynucleotide sequence
SEQ ID NO: 10
>4 dna: chromosome chromosome: GRCh38: 4:
122609508: 122621669: -1
GenBank RefSeq (mRNA): NM_021803; NM_001207006
GenBank RefSeq (protein): NP_001193935; NP_068575

What is claimed is:

1. An immune cell engineered to reduce or eliminate expression and/or function of an NR4A transcription factor in said immune cell.

2. An immune cell engineered to reduce or eliminate expression and/or function of a TOX transcription factor in said immune cell.

3. An immune cell engineered to reduce or eliminate expression and/or function of an NR4A and a TOX transcription factor in said immune cell.

4. An immune cell engineered to reduce or eliminate expression and/or function of an NR4A and a TOX transcription factor, band increase expression of IL-21 in said immune cell.

5. An immune cell engineered to inhibit expression and/or function of NFAT/AP-1 pathway in said immune cell.

6. The immune cell of claim 1, comprising the gene expression profile as shown in Table 1 and Table 2.

7. The immune cell of any one of claim 1, 3, 4 or 6, wherein the NR4A transcription factor is NR4A1 (Nur77), NR4A2 (Nurr1) or NR4A3 (NOR1).

8. The immune cell of claim 7, wherein the immune cell is engineered to reduce or eliminate expression and/or function of two or more of NR4A1 (Nur77), NR4A2 (Nurr1) and NR4A3 (NOR1).

9. The immune cell of any one of claim 2, 3, or 4, wherein the TOX transcription factor is TOX1, TOX2, TOX3 or TOX4.

10. The immune cell of claim 9, wherein the immune cell is engineered to reduce or eliminate expression and/or function of TOX1 and TOX2.

11. The immune cell of claim 9, wherein the immune cell is engineered to reduce or eliminate expression and/or function of two or more of TOX1, TOX2, TOX3 or TOX4.

12. The immune cell of claim 9, wherein the cell is engineered to reduce or eliminate expression and/or function of three or more of TOX1, TOX2, TOX3 or TOX4.

13. The immune cell of claim 4, wherein the immune cell is engineered to increase expression and/or function of IL-21 in said immune cell.

14. The immune cell of claim 5, wherein the immune cell is engineered to inhibit expression and/or function of NFAT/AP-1 pathway in said cell.

15. The immune cell of any one of claims 1 to 14, wherein the immune cell expresses a receptor that binds at least one tumor antigen or at least one antigen expressed by a pathogen.

16. The immune cell of any one of claim 15, wherein the tumor antigen comprises mesothelin, ROR1, or EGFRvIII.

17. The immune cell of any one of claims 1 to 16, wherein the immune cell is a T cell.

18. The immune cell of any one of claims 1 to 17, wherein the immune cell is a CD4 T cell, CD8 T cell or a Natural Killer (NK) T cell.

19. The immune cell of any one of claims 1 to 18, wherein the immune cell further comprises a suicide gene.

20. The immune cell of any one of claims 1 to 19, wherein the immune cell comprises a chimeric antigen receptor (CAR).

21. The immune cell of claim 20, wherein the chimeric antigen receptor (CAR) comprises: (a) an antigen binding domain; (b) a hinge domain; (c) a transmembrane domain; (d) and an intracellular domain.

22. The immune cell of claim 20 or 21, wherein the chimeric antigen receptor (CAR) comprises: (a) an anti-CD19 binding domain; (b) a hinge domain; (c) a CD28 or a CD8 α transmembrane domain; (d) one or more costimulatory regions selected from a CD28 costimulatory signaling region, a 4-1BB costimulatory signaling region, an ICOS costimulatory signaling region, and an OX40 costimulatory region; and (e) a CD3 zeta signaling domain.

23. The immune cell of claim 22, wherein the anti-CD19 binding domain of the CAR comprises a single-chain variable fragment (scFv) that specifically recognizes a humanized anti-CD19 binding domain.

24. The immune cell of claim 22 or 23, wherein the anti-CD19 binding domain scFv of the CAR comprises a heavy chain variable region and a light chain variable region.

25. The immune cell of claim 24, wherein the anti-CD19 binding domain of the CAR further comprises a linker polypeptide located between the anti-CD19 binding domain scFv heavy chain variable region and the anti-CD19 binding domain scFv light chain variable region.

26. The immune cell of claim 25, wherein the linker polypeptide of the CAR comprises a polypeptide of the sequence (GGGGS)n wherein n is an integer from 1 to 6.

27. The immune cell of any one of claims 20-26, wherein the CAR further comprises a detectable marker attached to the CAR.

28. The immune cell of any one of claims 20-26, wherein the CAR further comprises a purification marker attached to the CAR.

29. The immune cell of any one of claims 20-28, wherein the immune cell comprises a polynucleotide encoding the CAR, and optionally, wherein the polynucleotide encodes and anti-CD19 binding domain.

30. The immune cell of any one of claims 20-29, wherein the polynucleotide further comprises a promoter operatively linked to the polynucleotide to express the polynucleotide in said immune cell.

31. The immune cell of claim 29, wherein the polynucleotide further comprises a 2A self-cleaving peptide (T2A) encoding polynucleotide sequence located upstream of a polynucleotide encoding the anti-CD19 binding domain.

32. The immune cell of any one of claims 29-31, wherein the polynucleotide further comprises a polynucleotide encoding a signal peptide located upstream of a polynucleotide encoding the anti-CD19 binding domain.

33. The immune cell of claim 32, wherein the signal peptide encoding polynucleotide sequence of the CAR is a mouse Thy1.1 reporter.

34. The immune cell of any one of claims 20-33, wherein the polynucleotide sequence comprises SEQ ID NO:1.

35. The immune cell of any one of claims 20-33, wherein the polynucleotide encodes the amino acid sequence of SEQ ID NO:2.

36. The immune cell of claim 34 or 35, wherein the polynucleotide further comprises a vector comprising the isolated nucleic acid sequence comprising SEQ ID NO:1.

37. The immune cell of claim 36, wherein the vector is a plasmid.

38. The immune cell of claim 36, wherein the vector is a viral vector selected from the group of a retroviral vector, a lentiviral vector, an adenoviral vector, and an adeno-associated viral vector.

39. The immune cell of any one of claims 1-38, wherein the immune cell has been isolated from a subject.

40. The immune cell of any one of claim 39, wherein the subject has cancer.

41. The immune cell of claim 40, wherein the tumor antigen is expressed by a cell associated with the cancer.

42. The immune cell of any one of claim 39, wherein the subject has a pathogen infection, and optionally wherein the antigen is expressed by a cell infected with the pathogen.

43. A method of producing an engineered immune cell, the method comprising reducing or eliminating expression and/or function of an NR4A transcription factor in said immune cell.

44. A method of producing an engineered immune cell, the method comprising reducing or eliminating expression and/or function of a TOX transcription factor in said immune cell.

45. A method of producing an engineered immune cell, the method comprising reducing or eliminating expression and/or function of an NR4A and a TOX transcription factor in said immune cell.

46. A method of producing an engineered immune cell, the method comprising reducing or eliminating expression and/or function of an NR4A and a TOX transcription factor, and increasing the expression of IL-21 in said immune cell.

47. A method of producing an engineered immune cell, the method comprising inhibiting expression and/or function of NFAT/AP-1 pathway in said immune cell.

48. The method of any one of claim 43, 45, or 46, wherein the method comprises isolating an immune cell from a subject, reducing or eliminating expression and/or function of an NR4A transcription factor in said cell and culturing the immune cell under conditions that favor expansion and proliferation of the cell.

49. The method of any one of claim 44, 45, or 46, wherein the method comprises isolating an immune cell from a subject, reducing or eliminating expression and/or function of a TOX transcription factor in said cell and culturing the immune cell under conditions that favor expansion and proliferation of the cell.

50. The method of claim 47, wherein the method comprises isolating an immune cell from a subject, increasing expression and/or function of IL-21 in said cell and culturing the immune cell under conditions that favor expansion and proliferation of the cell.

51. The method of claim 48, wherein the method comprises isolating an immune cell from a subject, inhibiting expression and/or function of NFAT/AP-1 pathway in said cell and culturing the immune cell under conditions that favor expansion and proliferation of the cell.

52. The method of any one of claims 43-51, wherein the immune cell isolated from the subject binds a target antigen.

53. The method of claim 52, wherein the immune cell is a T cell.

54. The method of claim 52, wherein the immune cell is a CD4 T cell, CD8 T cell or a Natural Killer (NK) T cell.

55. The method of claim 52-54, wherein the target antigen is at least one tumor antigen or at least one antigen expressed by a pathogen.

56. The method of claim 55, wherein the tumor antigen comprises mesothelin, ROR1, or EGFRvIII.

57. The method of any one of claims 43-56, further comprising introducing into the cell a polynucleotide encoding a chimeric antigen receptor (polynucleotide CAR).

58. The method of claim 57, wherein the polynucleotide CAR comprises a polynucleotide encoding: (a) an antigen binding domain; (b) a hinge domain; (c) a transmembrane domain; (d) and an intracellular domain.

59. The method of claim 57 or 58, wherein the polynucleotide CAR comprises: (a) an anti-CD19 binding domain; (b) a hinge domain; (c) a CD28 or a CD8 α transmembrane domain; (d) one or more costimulatory regions selected from a CD28 costimulatory signaling region, a 4-1BB costimulatory signaling region, an ICOS costimulatory signaling region, and an OX40 costimulatory region; and (e) a CD3 zeta signaling domain.

60. The method of claim 59, wherein the anti-CD19 binding domain of the polynucleotide CAR encodes a single-chain variable fragment (scFv) that specifically recognizes a humanized anti-CD19 binding domain.

61. The method of claim 60, wherein the anti-CD19 binding domain scFv of the CAR encodes a heavy chain variable region and a light chain variable region.

62. The method of 60, wherein the anti-CD19 binding domain of the CAR further comprises a polynucleotide encoding linker polypeptide located between the anti-CD19 binding domain scFv heavy chain variable region and the anti-CD19 binding domain scFv light chain variable region.

63. The method of claim 62, wherein the polynucleotide encoding the linker polypeptide encodes the sequence (GGGGS)n wherein n is an integer from 1 to 6.

64. The method of any one of claims 57-63, wherein the polynucleotide further comprises a detectable marker.

65. The method of any one of claims 57-64, wherein the polynucleotide further comprises a polynucleotide encoding a purification marker.

66. The method of any one of claims 57-65, wherein the polynucleotide further comprises a promoter operatively linked to the polynucleotide to express the polynucleotide in said immune cell.

67. The method of immune cell of any one of claims 60-66, wherein the polynucleotide further comprises a 2A self-cleaving peptide (T2A) encoding polynucleotide sequence located upstream of the polynucleotide encoding the anti-CD19 binding domain.

68. The method of any one of claims 57-67, wherein the polynucleotide further comprises a polynucleotide encoding a signal peptide located upstream of a polynucleotide encoding the anti-CD19 binding domain.

69. The method of claim 68, wherein the polynucleotide encoding the signal peptide encodes a mouse Thy1.1 reporter.

70. The method of any one of claims 57-69, wherein the polynucleotide sequence comprises SEQ ID NO:1.

71. The method of any one of claims 57-70, wherein the polynucleotide encodes the amino acid sequence of SEQ ID NO:2.

72. The method of any one of claims 57-71, wherein the polynucleotide further comprises a vector.

73. The method of claim 72, wherein the vector is a plasmid.

74. The method of claim 73, wherein the vector is a viral vector selected from the group of a retroviral vector, a lentiviral vector, an adenoviral vector, and an adeno-associated viral vector.

75. A immune cell prepared by the method of any one of claims 57-74.

76. A substantially homogenous population of cells of any of claim 1 to 42 or 75.

77. A heterogeneous population of cells of any of claim 1 to 42 or 75.

78. A composition comprising a carrier and one or more of any of the cell of claim 1 to 42 or 75, or the population of cells of claim 76 or 77.

79. The composition of claim 7578, wherein the carrier is a pharmaceutically acceptable carrier.

80. The composition of claim 78 or 79, further comprising a cryoprotectant.

81. The immune cell of any one of claim 1 to 42 or 75, bound to a target cell.

82. A kit comprising vectors and instructions for the manufacture of the cell of any of claim 1 to 42 or 75, and optionally, instructions for their use diagnostically or therapeutically.

83. A method for stimulating immune cell-mediated immune response to a target cell population, the method comprising contacting the target cell population with the cell of any one of claim 1 to 42 or 75, the population of claim 76 or 77.

84. The method of claim 83, wherein the contacting is in vitro or in vivo.

85. The method of claim 84, wherein the contacting is in vivo and the target cell population is a population of cancer cells in a subject.

86. The method of claim 84, wherein the contacting is in vivo, and target cell population is a population of pathogen infected cells in a subject, and optionally wherein the cell of any one of claim 1 to 42 or 75, specifically bind a cell to the target cell population.

87. The method of claim 85, wherein the subject has, has had or is in need of treatment for cancer.

88. The method of claim 86, wherein the subject has, has had or is in need of treatment for a pathogen infection.

89. A method of treating cancer in a subject, the method comprising administering to the subject the cell of any one of claim 1 to 42 or 75, or the composition of claim 76 or 77.

90. A method of providing anti-tumor immunity in a subject, the method comprising administering to the subject the cell of any one of claim 1 to 42 or 75, or the composition of claim 76 or 77.

91. The method of claim 89 or 90, wherein the subject is a mammal.

92. The method of claim 91, wherein the subject is a human.

93. A method of treating a subject having a disease, disorder or condition associated with an elevated expression of a tumor antigen, the method comprising administering to the subject the cell of any one of claim 1 to 42 or 75, or the composition of claim 76 or 77.

94. A method of treating a pathogen infection in a subject, the method comprising administering to the subject the cell of any one of claim 1 to 42 or 75, or the composition of claim 76 or 77.

95. A method of providing immunity to the pathogen infection in a subject, the method comprising administering to the subject the cell of any one of claim 1 to 42 or 75, or the composition of claim 76 or 77.

96. The method of any one of claims 93 to 95, wherein the subject is a mammal.

97. The method of claim 96, wherein the subject is a human.
